(19) United States
(12) Patent Application Publication
(10) Pub. No.: US 2024/0270971 A1
(43) Pub. Date: Aug. 15, 2024

(54) UV-ABSORBING POLYMERS, COMPOSITIONS AND USES THEREOF

(71) Applicant: Beckman Coulter, Inc., Brea, CA (US)

(72) Inventors: Arunkumar Easwaran, Miami, FL (US); Massimiliano Tomasulo, Miami, FL (US); Sergei Gulnik, San Jose, CA (US); Boi Hoa San, Miami, FL (US)

(21) Appl. No.: 18/558,489

(22) PCT Filed: May 3, 2022
(86) PCT No.: PCT/US2022/027520

§ 371 Date: Nov. 1, 2023

Related U.S. Application Data
(60) Provisional application No. 63/183,862, filed on May 4, 2021.
(60) Provisional application No. 63/306,946, filed on Feb. 4, 2022.

(51) Int. Cl. C09B 69/10; G01N 33/58

(52) U.S. Cl. CPC C09B 69/109; G01N 33/582; G01N 2458/00

(57) ABSTRACT

The present disclosure provides UV-absorbing polymer dyes and methods for detecting an analyte in a sample by using a binding partner conjugated to a UV-absorbing polymer dye. Compositions comprising UV-absorbing polymer dyes, UV-absorbing tandem dyes, or quenched UV polymer dyes are provided.

This application is being filed on May 3, 2022, as a PCT International Patent application and claims the benefit of and priority to U.S. Provisional Application Ser. No. 63/183,862, filed May 4, 2021, and U.S. Provisional Application Ser. No. 63/306,946, filed Feb. 4, 2022, each of which is incorporated by reference herein in its entirety.

### BACKGROUND

Polymers that absorb ultra-violet (“UV”) light can be used in a variety of biological applications by generating signals which can be monitored in real time and provide simple and rapid methods for the detection of biological targets and events.

However, many of the previously reported UV-absorbing polymers are highly hydrophobic. Many UV-absorbing polymeric dyes are not useful under aqueous conditions due to poor solubility, brightness, and broadening of the spectra. Therefore, the available arsenal of UV-absorbing polymeric dyes for biological applications, including for the detection of analytes, is deficient.

### SUMMARY OF THE DISCLOSURE

The disclosure provides novel UV excitable (e.g., 355 nm) polymer dyes, polymer-tandem dyes, polymer dye conjugates and polymer-tandem dye conjugates. It also provides methods of detecting an analyte in a sample using the polymer dyes and polymer dye conjugates by, for example, flow cytometry. Compositions comprising UV polymer dyes, UV polymer-tandem dyes, UV polymer conjugates and/or U polymer-tandem dye conjugates are also provided.

The disclosure provides a UV-absorbing polymer having the structure of Formula I:

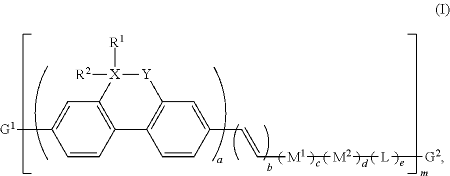

wherein each X is independently selected from the group consisting of C and Si; each Y is independently selected from the group consisting of a bond, CR¹R², CHR¹, CHR², SiHR², SiHR¹, and SiR¹R², and when Y is a bond X is directly bonded to both rings; each R¹ is independently selected from the group consisting of a water-solubilizing moiety, a linker moiety, alkyl, alkene, alkyne, cycloalkyl, haloalkyl, (hetero)aryloxy, (hetero)arylamino, aryl, heteroaryl, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide,

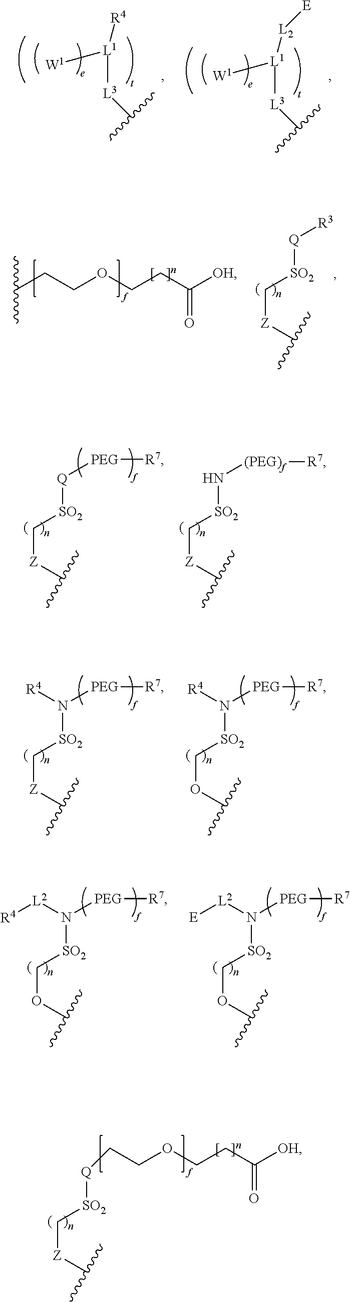

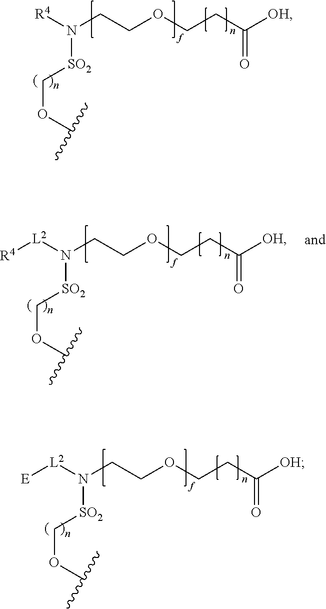

each R² is independently selected from the group consisting of a water-solubilizing moiety, a linker moiety, H, alkyl, alkene, alkyne, cycloalkyl, haloalkyl, alkoxy, (hetero)aryloxy, aryl, heteroaryl, (hetero)arylamino, a PEG group, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

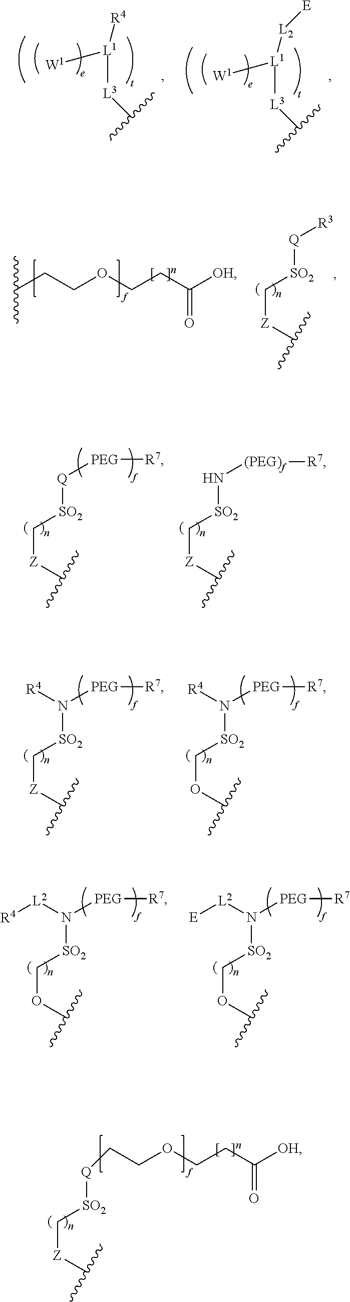

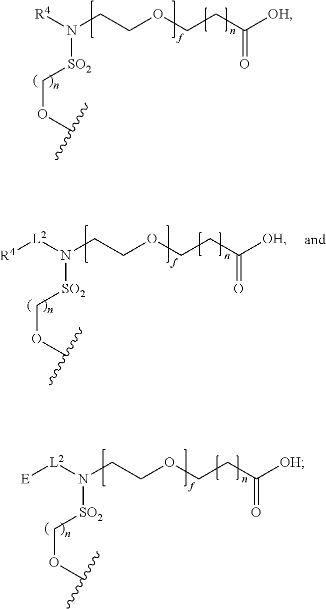

each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, and a PEG group; each Z is independently selected from the group consisting of CH₂, CHR⁴, O, NH, and NW; each Q is independently selected rom the group consisting of a bond, NH, NR⁴, C₁-C₁₂ alkylene, CHR⁴, and CH₂; each R⁴ is independently selected the group consisting of H, a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OR⁹ wherein R⁹ is C₁-C₈ alkyl, each x′ is independently an integer from 0-20 and each y′ is independently an integer from 0-50, Z—(CH₂)ₙ—SO₂-Q-R³, a C₂-C₁₈ (hetero)aryl group, amide, amine, carbamate, carboxylic acid, carboxylate ester, maleimide, activated ester, N-hydroxysuccinimidyl, hydrazine, hydrazone, azide, aldehyde, thiol, and protected groups thereof; each W¹ is independently a water-solubilizing moiety; L¹, L², and L³ are each independently selected linker moieties; each E is independently selected from the group consisting of a chromophore, linked chromophore, a functional moiety, a linked functional moiety, a substrate, a linked substrate, a binding partner, and a linked binding partner; each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and —OC₁-C₁₂ hydroxy; at least one of R¹, R², R³, or R⁴ comprises a water-solubilizing moiety; each M¹ is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted; each M² is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than M¹, and wherein M² and M¹ are evenly or randomly distributed along the polymer main chain; each optional linker L is an aryl or heteroaryl group evenly or randomly distributed along the polymer main chain wherein L is optionally substituted; G¹ and G² are each independently selected from the group consisting of an unmodified polymer terminus and a modified polymer terminus, optionally conjugated to E; a, c, d, and e define the mol % of each unit within the structure which each can be evenly or randomly repeated along the polymer main chain and where a is a mol % from 10 to 100%, c is a mol % from >0 to 90%, each d is a mol % from 0 to 90%, and each e is a mol % from 0 to 25%; each b is independently 0 or 1; each f is independently an integer from 0 to 50; m is an integer from 1 to about 10,000; each n is independently an integer from 1 to 20; s is 1 or 2; and t is 0, 1, 2, or 3.

The UV-absorbing polymer dye having the structure of Formula I may have a near ultraviolet excitation spectrum and/or absorbance maximum in a range of from about 300 nm to about 400 nm, or from about 350 nm to about 400 nm. The near UV-absorbing polymer dye having the structure of Formula I may be a water-soluble polymer. In some cases, the UV-absorbing polymer comprises at least one water-solubilizing group.

The units in the UV-absorbing polymer structure presented in Formula I can occur in any suitable order, including randomly, within the polymer backbone, such as the same or different order as shown in Formula I. The M¹ and M² units can be distributed randomly in alternate positions throughout the polymer backbone. The M¹ and M² units can be present in any suitable molar ratio to one another in the IV-absorbing polymer. Each L may independently be substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule, or binding partner.

In various aspects, the present disclosure provides a UV-absorbing polymer having the structure of Formula I:

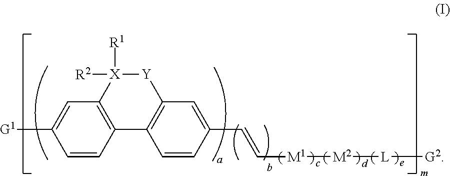

Each X is independently selected from C and Si. Each Y is independently selected from a bond, CR¹R², CHR₁, CHR₂, and SiR¹R², and when Y is a bond X is directly bonded to both rings. Each R¹ is independently selected from polyethylene glycol (PEG), a PEG group, ammonium alkyl salt, ammonium alkyloxy salt, ammonium oligoether salt, sulfonate alkyl salt, sulfonate alkoxy salt, sulfonamido oligoether, —Z—(CH₂)ₙ—SO₂-Q-R³, —Z—(CH₂)ₙ—SO₂—NH—R³ and —Z—(CH₂)ₙ—SO₂—N(R⁴)—R³. Each R² is independently selected from H, alkyl, alkene, alkyne, cycloalkyl, haloalkyl, alkoxy, (hetero)aryloxy, aryl, (hetero)arylamino, a PEG group, ammonium alkyl salt, ammonium alkyloxy salt, ammonium oligoether salt, sulfonate alkyl salt, sulfonate alkoxy salt, sulfonate oligoether salt, sulfonamido oligoether, —Z—(CH₂)ₙ—SO₂-Q-R³, —Z—(CH₂)ₙ—SO₂—NH—R³ and —Z—(CH₂)ₙ—SO₂—N(R⁴)—R³. Each R³ is independently selected from H, alkyl, alkene, alkyne, cycloalkyl, haloalkyl, alkoxy, (hetero)aryloxy, aryl, (hetero)arylamino, and a PEG group. Each Z is independently selected from CH₂, CHR⁴, O, NR⁴, and NH. Each Q is independently selected from a bond, NH, NR⁴, C₁-C₁₂ alkylene, CHR⁴, and CH₂. Each R⁴ is independently selected from a chromophore, a linked chromophore, a functional group, a linked functional group, a substrate, a linked substrate, a binding partner, a linked binding partner, halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₁₈ (hetero)aryloxy, C₂-C₁₈ (hetero)arylamino, (CH₂)_{x′}(OCH₂—CH₂)_{y′}OCH₃ wherein each x′ is independently an integer from 0-20 and each y′ is independently an integer from 0-50, —Z—(CH₂)ₙ—SO₂-Q-R³, and a C₂-C₁₈ (hetero)aryl group. Each modifying unit M¹ and M² can be independently selected from an arylene or heteroarylene capable of altering the band gap of the polymer. Each M¹ is independently selected from an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl optionally substituted. Each M² is independently selected from an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than M¹. Each linker L is an aryl or heteroaryl group evenly or randomly distributed along the polymer main chain. Each L can be substituted with one or more pendant chains terminated with a functional group selected from, for example, amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule, or binding partner.

G¹ and G² are each independently selected from the group consisting of an unmodified polymer terminus and a modified polymer terminus. In some examples, the variables G¹ and G² may each independently be selected from hydrogen, halogen, alkyne, halogen substituted aryl, silyl, diazonium salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), or optionally substituted fluorene, wherein the optionally substituted aryl, heteroaryl, fluorene, or DHP may be substituted with one or more pendant chains terminated with a functional group selected, for example, from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof, for conjugation to a substrate or binding partner.

The variables a, c, d, and e define the mol % of each unit within the structure which each can be evenly or randomly repeated along the polymer main chain and where a is a mol % from 10 to 100%, c is a mol % from >0 to 90%, each d is a mol % from 0 to 90%, and each e is a mol % from 0 to 25%. Each b is independently 0 or 1. The variable m is an integer from 1 to about 10,000. Each n is independently an integer from 1 to 20.

In some examples, M¹ may independently be selected from a mono-, di-, tri-, or tetra-R⁴- and/or trifluoromethyl-substituted arylene that is optionally further substituted; a mono-, di-, tri-, or tetra-R⁴- and/or trifluoromethyl-substituted heteroarylene that is optionally further substituted; a mono-, di-, tri-, or tetra-R⁴- and/or trifluoromethyl-substituted 9,10-dihydrophenanthrene that is optionally further substituted; and a binaphthyl that is optionally substituted. In some examples, each M² may independently selected from a mono-, di-, tri-, or tetra-R⁴- and/or trifluoromethyl-substituted arylene that is optionally further substituted; a mono-, di-, tri-, or tetra-R⁴- and/or trifluoromethyl-substituted heteroarylene that is optionally further substituted; a mono-, di-, tri-, or tetra-R⁴- and/or trifluoromethyl-substituted 9,10-dihydrophenanthrene that is optionally further substituted; and a binaphthyl that is optionally substituted. In some examples, M² has a different structure than M¹.

Each linker moiety may independently be selected from the group consisting of L, L¹, L², and L³.

In some examples, the linking moieties L¹, L², and L³ may independently be, but are not limited to, a covalent bond, C₁₋₈ alkylene, 2- to 8-membered heteroalkylene. In some embodiments, the linker is a single atom, a linear chain, a branched chain, a cyclic moiety. In some embodiments, the linker is chain of between 2 and 100 backbone atoms (e.g., carbon atoms) in length, such as between 2 and 50 backbone atoms in length or between 2 and 20 atoms backbone atoms in length. In certain cases, one, two, three, four or five or more carbon atoms of a linker backbone can be optionally replaced with sulfur, nitrogen, or oxygen. The bonds between backbone atoms can be saturated or unsaturated; typically, not more than one, two, or three unsaturated bonds will be present in a linker backbone. The linker can include one or more substituent groups (e.g., an alkyl group or an aryl group). A linker can include, without limitation, oligo(ethylene glycol); ethers; thioethers; tertiary amines; and alkylene groups (i.e., divalent alkyl radicals), which can be straight or branched. The linker backbone can include a cyclic group, for example, a divalent aryl radical, a divalent heterocyclic radical, or a divalent cycloalkyl radical, where 2 or more atoms, e.g., 2, 3 or 4 atoms, of the cyclic group are included in the backbone.

In some examples, L¹ comprises a sulfonamide, a sulfonimide, a sultam, a disulfinamide, an amide, a phosphonamide, a phosphonamidate, a phosphinamide, a selenoonamide, a seleninamde, or a secondary amine. In some embodiments, L¹ comprises a sulfonamide, an amide, a phosphonamide, or a secondary amine. In some cases, L¹ is a linker moiety optionally terminated with L²-E. In some cases, L² comprises a linear or branched, saturated or unsaturated C₁₋₃₀ alkylene group; wherein one or more carbon atoms in the C₁₋₃₀ alkylene group is optionally and independently replaced by O, S, NR^{a}; wherein two or more groupings of adjacent carbon atoms in the C₁₋₃₀ alkylene are optionally and independently replaced by —NR^{a}(CO)— or —(CO)NR^{a}—; and wherein each R^{a} is independently selected from 1-1 and C₁₋₆ alkyl; and wherein each R^{a} is independently selected from H and C₁₋₆ alkyl.

In some examples, L² is a linker moiety optionally terminated with a functional moiety selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to a chromophore, substrate, or a binding partner.

In some examples, L³ is selected from the group consisting of a covalent bond, C i-s alkylene, 2- to 8-membered heteroalkylene (e.g., a divalent alkoxy linker such as —O-alkyl), (C₃₋₈ cycloalkylene, C₆₋₁₀ arylene, 5- to 12-membered heteroarylene, 5- to 12-membered heterocyclylene, an amine, —NHC(O)L^{a}-, —C(O)NHL^{a}-, —C(O)L^{a}-, and combinations thereof, wherein L^{a} is selected from the group consisting of C₁₋₈ alkylene and 2- to 8-membered heteroalkylene.

In some cases, L¹, L², and L³ together form the following:

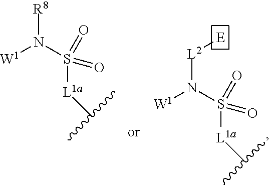

wherein R⁸ is R⁴, hydrogen, or an amine protecting group; and
L^{1a} is a linker moiety. In some cases, L^{1a} is selected from the group consisting of a covalent bond, C₁₋₈ alkylene, C₁₋₈ alkoxy, 2- to 8-membered heteroalkylene (e.g., a divalent alkoxy linker), C₃₋₈ cycloalkylene, C₆₋₁₀ arylene, 5- to 12-membered heteroarylene, 5- to 12-membered
heterocyclylene, —NHC(O)L^{a}-, —CC(O)NHL^{a}-, —C(O)L^{a}-, and combinations thereof. In some embodiments, L^{1a} is selected from the group consisting of a covalent bond, C₁₋₈ alkylene, 2- to 8-membered heteroalkylene, —NHC(O)L^{a}-, —C(O)NHL^{a}-, and —C(O)L^{a}-.

In some examples, L³ is a trivalent arylalkyl moiety having: a first point of attachment to a first L¹ moiety (or a first L^{1a} moiety); a second point of attachment to a second L¹ moiety (or a second L^{1a} moiety); and a third point of attachment to an A monomer. For example, some embodiments of the disclosure provide conjugated polymers having two or more E groups, such as chromophores, attached as shown in Formula XIII:

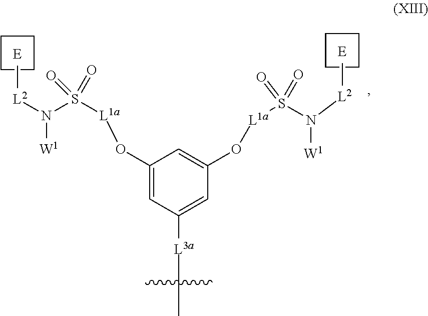

wherein
L^{3a} is selected from the group consisting of a covalent bond, C₁₋₈ alkylene, 2- to 8-membered heteroalkylene, —NHC(O)L^{a}-, —C(O)NHL^{a}-, and —C(O)L^{a}-;
L^{1a} is C₁-8 alkylene or 2- to 8-membered heteroalkylene; W¹ is a water-solubilizing moiety;
and the wavy line is the point of the attachment to the A monomer; and each E and L² is independently as described herein above.

In some examples, each E is an independently selected chromophore (e.g., and independently selected fluorophore). In some embodiments, all of the E moieties in the polymer have the same fluorophore structure. In some embodiments, all of the E moieties in the polymer have a different fluorophore structure.

In some examples, W¹ is a water-solubilizing moiety selected from ethylene glycol, PEG groups, carboxy groups including but not limited to carboxylic acids and carboxylates, polyvinyl alcohols, glycols, peptides, polyphosphates, polyalcohols, sulfonates, phosphonates, boronates, amines, ammoniums, sulfoniums, phosphonium, alcohols, polyols, oxazolines, zwitterionic derivatives, carbohydrates, nucleotides, polynucleotides, substituted PEG groups, substituted carboxy groups including but not limited to substituted carboxylic acids and substituted carboxylates, substituted glycols, substituted peptides, substituted polyphosphates, substituted polyalcohols, substituted sulfonates, substituted phosphonates, substituted boronates, substituted amines, substituted ammoniums, substituted sulfoniums, substituted phosphonium, alcohols, substituted zwitterionic derivatives, substituted carbohydrates, substituted nucleotides, substituted polynucleotides, or combinations thereof.

In some cases, W¹ comprises one or more ethylene glycol monomers. In some cases, W¹ comprises a PEG group.

In some examples, the variables G¹ and G² may each independently be selected from hydrogen, halogen, alkyne, halogen substituted aryl, silyl, diazonium salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), or optionally substituted fluorene, wherein the optionally substituted aryl, heteroaryl, fluorene, or DHP may be substituted with one or more pendant chains terminated with a functional group, for example, selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof, for conjugation to a substrate or binding partner.

In some examples, each optional L is a linker moiety independently selected from the group consisting of:

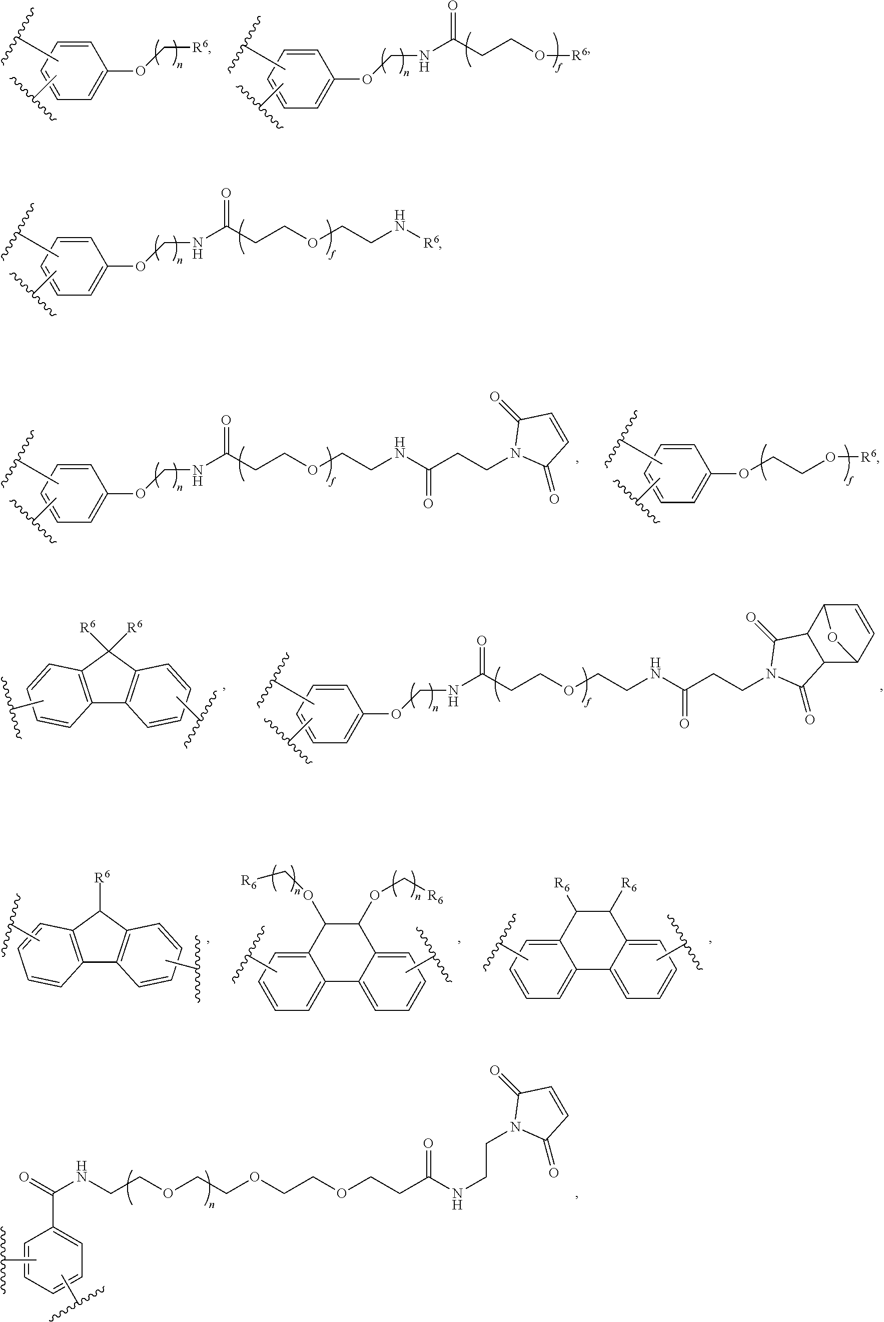

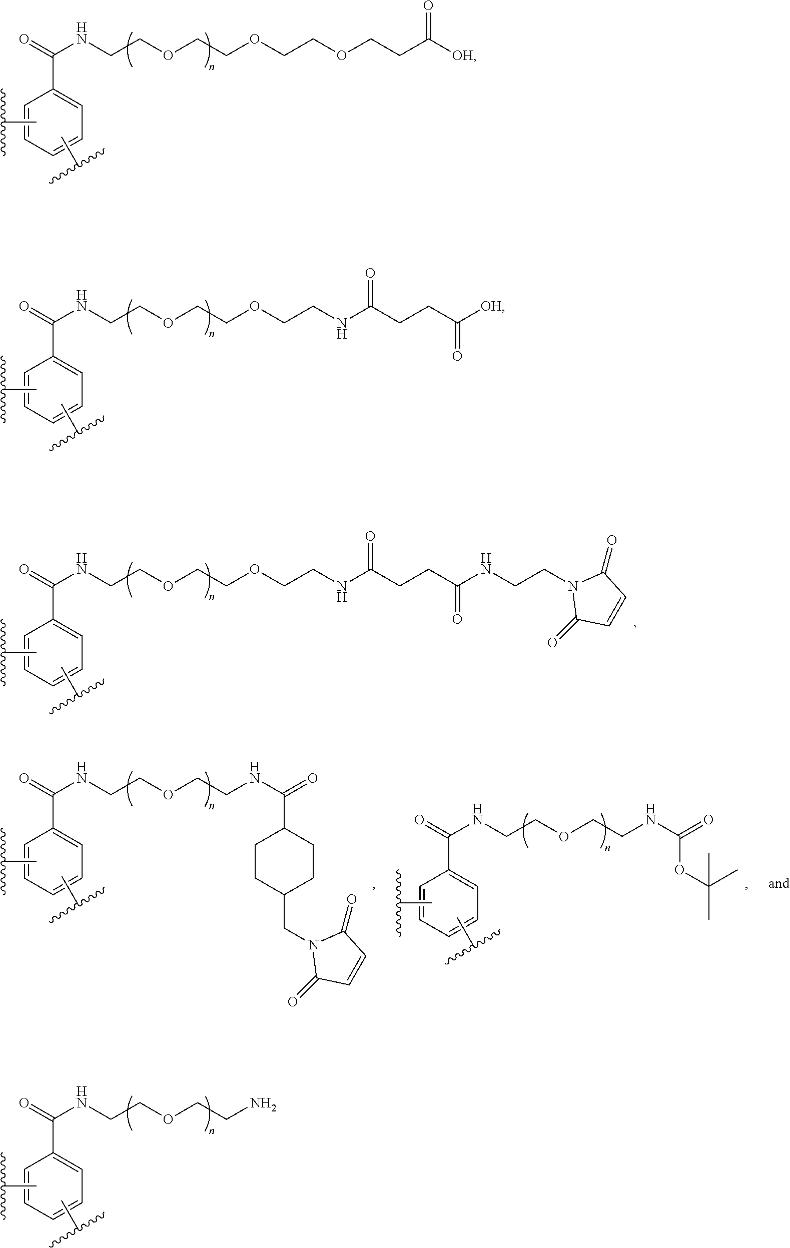

wherein,
each R⁶ is independently selected from the group consisting of H, OH, SH, NHCOO-t-butyl, (CH₂)ₙCOOH, (CH₂)ₙCOOCH₃, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂ halo alkyl, C₁-C₁₂(hetero)aryl, C₁-C₁₂(hetero)arylamino, optionally substituted benzyl, halogen, hydroxyl, C₁-C₁₂ alkoxy, (OCH₂CH₂)_{f}OCH₃,

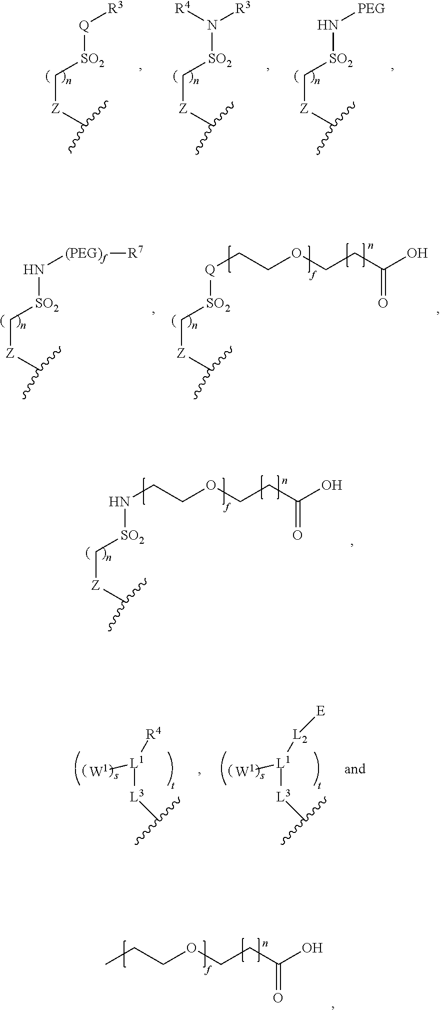

wherein W¹ is a water-solubilizing moiety; and each of R³, R⁴, R⁷, Z, Q, f, n, s, and t are each as described above.

In some examples, the disclosure provides a UV-absorbing polymer according to Formula XIV:

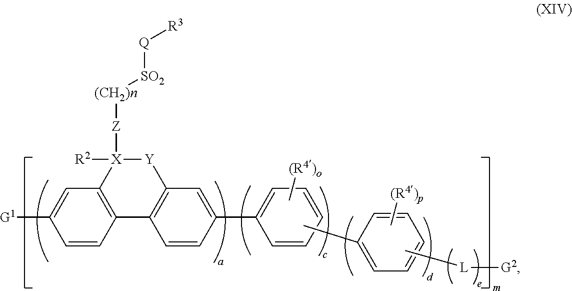

wherein
each of R², R³, G¹, G², L, Q, X, Y, Z, a, b, c, e, n, and m is independently as described herein;
each R^{4′} is independently selected from F, Cl, —CH₃, —CF₃, and —(OCH₂CH₂)_{f}OR⁹; each R^{4″} is independently selected from F, Cl, —CH₃, —CF₃, and —(OCH₂CH₂)_{f}OR₉; R⁹ is C₁-C₈ alkyl; f is 0 to 50, or 10-20; each o is independently an integer selected from 1, 2, 3, or 4; and each p is independently an integer selected from 1, 2, 3, or 4. The units in the polymer structure presented in Formula XIV can occur in any suitable or random order within the polymer backbone, such as the same or different order as shown in Formula XIV. The M¹ and M² units can be distributed randomly in alternate positions throughout the polymer backbone. In some examples, the LV-absorbing polymer according to Formula XIV has a near ultraviolet excitation spectrum and/or absorbance maximum in a range of from 300 nm to 400 nm, or from 350 nm to 400 nm.

In some cases, the present disclosure provides a co-polymer comprising a structure of Formula (I) as previously defined.

In some embodiments, the present disclosure provides polymer tandem dye comprising a UV-absorbing polymer dye having the structure of Formula (I) as previously defined and a signaling chromophore covalently linked to the UV-absorbing polymer dye in energy-receiving proximity therewith.

In some cases, the present disclosure provides a labelled binding partner, comprising a UV-absorbing polymer dye having the structure of Formula (I) as previously defined a binding partner covalently linked to the UV-absorbing polymer dye.

In various aspects, the present disclosure provides a method for detecting an analyte in a sample. The method includes contacting a sample that is suspected of containing the analyte with a binding partner capable of interacting with the analyte conjugated to a UV-absorbing polymer according to the disclosure (a polymer conjugate). In some cases, the UV-absorbing polymer conjugate comprises the structure of Formula I:

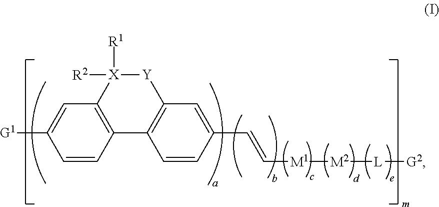

wherein X, Y, G¹, G², R¹, R², M¹, M², L, a, b, c, d, and e are each individually as defined in the present disclosure.

In some cases, X is independently selected from C and Si; each Y is independently selected from a bond, CHR₁, CHR₂, CR¹R², and SiR¹R², and when Y is a bond X is directly bonded to both rings. Each R¹ is independently selected from polyethylene glycol (PEG), a PEG group, ammonium alkyl salt, ammonium alkyloxy salt, ammonium oligoether salt, sulfonate alkyl salt, sulfonate alkoxy salt, sulfonamido oligoether, a linked chromophore, —Z—(CH₂)ₙ—SO₂-Q-R³, —Z—(CH₂)ₙ—SO₂—NH—R³ and —Z—(CH₂)ₙ—SO₂—N(R⁴)—R³. Each R² is independently selected from H, alkyl, alkene, alkyne, cycloalkyl, haloalkyl, alkoxy, (hetero)aryloxy, aryl, (hetero)arylamino, a PEG group, ammonium alkyl salt, ammonium alkyloxy salt, ammonium oligoether salt, sulfonate alkyl salt, sulfonate alkoxy salt, sulfonate oligoether salt, sulfonamido oligoether, and —Z—(CH₂)ₙ—SO₂-Q-R³. Each R³ is independently selected from H, alkyl, alkene, alkyne, cycloalkyl, haloalkyl, alkoxy, (hetero)aryloxy, aryl, (hetero)arylamino, and a PEG group. Each Z is independently selected from CH₂, CHR⁴, O, NH, and NR⁴. Each Q is independently selected from a bond, NH, NR⁴, CHR⁴, C₁-C₁₂ alkylene, and CH₂. Each R⁴ is independently selected from a chromophore, halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OCH₃ wherein each x′ is independently an integer from 0-20 and each y′ is independently an integer from 0-50, —Z—(CH₂)ₙ—SO₂-Q-R³, and a C₂-C₁₈ (hetero)aryl group. Each modifying unit M¹ and M² can be independently selected from an arylene or heteroarylene capable of altering the band gap of the polymer. Each M¹ is independently selected from an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted. Each M² is independently selected from an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than M. Each linker L is an aryl or heteroaryl group evenly or randomly distributed along the polymer main chain and that is substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule or binding partner.

In some examples, the variables G¹ and G² may each independently be selected from hydrogen, halogen, alkyne, halogen substituted aryl, silyl, diazonium salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), or optionally substituted fluorene, wherein the optionally substituted aryl, heteroaryl, fluorene, or DHP may be substituted with one or more pendant chains terminated with a functional group, for example, selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof, for conjugation to a substrate or binding partner.

The variables a, c, d, and e define the mol % of each unit within the structure which each can be evenly or randomly repeated along the polymer main chain and where a is a mol % from 10 to 100%, c is a mol % from >0 to 90%, each d is a mol % from 0 to 90%, and each e is a mol % from 0 to 25%. Each b is independently 0 or 1. The variable m is an integer from 1 to about 10,000. Each n is independently an integer from 1 to 20. The binding partner is capable of interacting with the analyte or a target-associated biomolecule.

The UV-absorbing polymers of the present disclosure can provide good absorption in the near ultraviolet (UV) region of the spectrum (e.g., 350-400 nm, or 355-375 nm), e.g., at ˜355 nm, with no excitation at 405 nm or decreased excitation at ˜405 nm as compared to other polymers that provide absorption at ˜355 nm which reduces/eliminates spillover in the pacific blue channel (450±25 nm). The LV-absorbing polymers of the present disclosure can have good absorption at 355 nm and 375 nm, and therefore can be used with 355 nm and 375 nm laser-equipped instruments.

The disclosure provides a composition comprising a UV-absorbing polymer dye, DV-absorbing tandem polymer dye, or quenched V polymer dye, and a nonionic surfactant for reducing or preventing non-specific interactions between polymer dye conjugates. The UV-absorbing polymer dye, UV-absorbing tandem polymer dye, or quenched UV polymer dye may be a polymer dye conjugate. The V-absorbing polymer dye, UV-absorbing tandem polymer dye, or quenched V polymer dye may be a water-soluble DV-absorbing polymer dye. The UV-absorbing polymer dye, UV-absorbing tandem polymer dye, or quenched LUV polymer dye may be a polymer dye according to the disclosure.

A kit is also provided comprising the composition according to the disclosure, wherein the kit comprises a container comprising the composition; and optionally at least one or a plurality of fluorescent polymer dye conjugates. The kit may comprise the composition in one container; and the at least one or plurality of fluorescent polymer dye conjugate in separate containers.

### BRIEF DESCRIPTION OF THE FIGURES

The drawings illustrate generally, by way of example, but not by way of limitation, various embodiments of the present disclosure.

FIG. 1 illustrates a comparison of the signal-to-noise ratio at 405 nm channel of different lots (B, C, D) of UV-absorbing polymers according to the disclosure conjugated with CD4 antibody compared to competitive BLV395-CD4 antibody conjugate (A) (Becton Dickinson Biosciences) following laser excitation at 355 nm. IV polymer dye conjugates according to the disclosure exhibit improved S/N ratios compared to competitive product.

FIG. 2 illustrates a comparison of the signal-to-noise ratio at 740/40 nm channel of inventive UV polymer-DY704 tandem dye CD4 antibody conjugate in accordance with various aspects of the present disclosure compared with competitive BUV737-CD4 conjugate after laser excitation at 355 nm.

FIG. 3 shows flow cytometry analysis (FCA) dot plots of stained and lysed blood samples in presence of various concentrations of UV-absorbing polymer with or without 1% PF-68. Upper left panel shows blood only without additives, upper right panel shows addition of 10 ug quenched polymer 3, middle row four panels (left to right) show addition of UV-absorbing polymer at 2.5 ug, 5 ug, 10 ug, and 20 ug, lower four panels (left to right) show addition of UV-absorbing polymer at, 2.5 ug, 5 ug, 10 ug, and 20 ug with 1% PF-68. MFI values of monocytes indicate that UV polymer (2.5 ug-20 ug/test) with or without 1% PF-68 did not show strong non-specific binding to cells as compared with control cells (Blood only panel, upper left).

FIG. 4 shows FCA dot plots illustrating the effect of UV-absorbing polymer and other components of staining buffer composition in stained and lysed human whole blood samples with a mixture of two polymer dye conjugates: CD20-UV excitable polymer dye (UVEPD) and CD4-UV408, both according to the disclosure. Y-axis is FL1 channel and X-axis is UV405 channel. Stained cells in the presence of combined UV polymer (5-20 μg/test)+1% PF-68 (lower 3 panels) showed less spillover than the controls including no buffer added sample (upper left panel), 1% PF-68 (upper middle panel), and UV polymer alone (upper right panel). The values in each panel indicate the MFI values.

FIG. 5 shows FCA dot plots illustrating the effect of UV-absorbing polymer and other components of staining buffer composition in stained and lysed human whole blood samples with a mixture of two types of polymer dye conjugates: CD20-SN v428 (Beckman Coulter Life Sciences) and CD4-BV650 (Becton Dickinson Biosciences). Y-axis is V450-PB channel and X-axis is V660 channel. Stained cells in the presence of combined UV polymer (5-20 μg/test)+1% PF-68 (lower left, lower middle, and lower right panels) showed better separation than that of the controls including no buffer added sample (upper left), 1% PF-68 (upper middle), and UV polymer alone (10 ug/test; tipper right). The values in each panel indicate the MFI values.

FIG. 6 shows FCA dot plots of stained and lysed whole blood cells illustrating effect of different concentrations of nonionic surfactant concentration (0.1-1% PF-68/test) in presence of 10 ug UV polymer according to the disclosure in staining buffer compositions with a mixture of two polymer dye conjugates: CD20-SN v428 (Beckman Coulter Life Sciences) and CD4-BV650 (Becton Dickinson Biosciences). Y-axis is V450-PB channel and X-axis is V660 channel. Stained cells in the presence of combined UV polymer (10 ug/test)+PF-68 in different concentrations (0.1-1%/test) (lower left, lower middle and lower right panels) showed better separation than that of the controls including no buffer added sample (upper left panel), 1% PF-68 (upper middle panel), and UV polymer alone (10 ug/test; upper right panel). The values in each panel indicate the MFI values.

FIG. 7 shows emission spectra of three quenched UV polymers according to the disclosure after excitation at 355 nm. The ratio of quencher to polymer (D/P) was determined to be 2.5, 5, and 10. The quantum yield (QY) at 405 nM of the quenched polymer 1 (D/P=2.5), quenched polymer 2 (D/P=5.0), and quenched polymer 3 (D/P=10) are 0.072, 0.030, and 0.003, respectively. The quantum yield of the unquenched polymer is 0.739

FIG. 8 shows FCA dot plots of stained and lysed blood cells using a mixture of two different commercial polymer dye conjugates: CD20-SN v428 (Beckman Coulter Life Sciences) and CD4-BV650 (BD Biosciences). Y-axis is V450-P3 channel and X-axis is V660 channel. Stained cells without additives (lower left panel), with 1% PF-68 (upper left panel), with combined 10 ug UV polymer+1% PF-68 (middle left panel), with quenched polymer 1 (QY=0.072), at 5 ug, 10 ug or 20 ug/test with 1% PF-68 (second from left upper, middle and lower panels, respectively), with quenched polymer 2 (QY=0.03) at 5 ug, 10 ug or 20 ug/test with 1% PF-68 (second from right upper, middle and lower panels, respectively), and with quenched polymer 3 (QY=0.003) at 5 ug, 10 ug or 20 ug/test with 1% PF-68 (right upper, middle and lower panels, respectively). Stained cells in the presence of test staining buffer compositions including combined UV polymer (10 μg/test)+1% PF-68 (middle left panel) or stained cells in the presence of the quenched polymers 1, 2, and 3 at 5 ug, 10 ug or 20 ug/test, with 1% PF-68 (right three columns, upper, middle, lower panels, respectively) each showed better separation than control without additives (lower left panel). The values in each panel indicate the MFI values.

FIG. 9 shows FCA dot plots of stained and lysed cells with a mixture of CD4-BV650 (BD Biosciences) and CD19-SNv428 (Beckman Coulter Life Sciences). Y-axis is V450-PB channel and X-axis is V660 channel. Stained cells without buffer (left panel), with 0.1% PF-68 (second from left panel), 0.5% PF-68 (second from right panel), and 1% PF-68 (wt/vol) (right panel). The presence of various PF-68 concentrations (0.1-1% wt/vol) is associated with decreased non-specific interactions in the mixture as evidenced by improved separation compared to without PF-68.

FIG. 10 shows FCA dot plots of stained and lysed cells with a mixture of two polymer dye conjugates: CD4-Violet excitable polymer dye 1 (CD4-VEPD 1) and CD19-Violet excitable polymer dye 2 (CD19-VEPD 2) (both from Beckman Coulter Life Sciences). Y-axis is FL3 channel and X-axis is FL2 channel. Stained cells without buffer (Top panel), with combined staining buffer (composition of UV polymer+PF-68) and Empigen in various concentrations (0, 0.03%, 0.05%, and 0.07%) (middle panels: left, second from left, second from right, and right, respectively). The presence of various concentrations of Empigen in staining buffer do not affect the performance of staining buffer. Bottom panels: MFI values of monocytes indicate that with the addition of various concentrations of Empigen to staining buffer (bottom panels: from left to right) show decreased non-specific binding to cells as compared with control sample (bottom left panel).

FIG. 11 shows an exemplary partial scheme for preparing a UV-absorbing polymer according to the disclosure illustrating wherein two different modifying units M¹ (e.g., a difluorophenylene) and M² (e.g., a trifluorphenylene) are distributed randomly in alternate positions to the repeating DHP units.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the terms “moiety” and “group” are used interchangeably.

Unless otherwise specified, the term phrase “room temperature” refers to 18 to 27° C.

Unless otherwise specified, the term “wt percent” or “wt %” refers to weight percent/vol.

The phrases “ready to use reagent”, “ready to use reagent composition”, “working concentration reagent”, and “working concentration reagent composition” refer to a staining buffer composition produced at about 1× working concentration appropriate for use, for example, in a mixture of polymer dye conjugates for staining a biological sample for flow cytometry analysis (FCA).

The phrase “protecting group” (also referred to as “protected group”) refers to a reversibly formed derivative of an existing functional group in a molecule attached to decrease reactivity so that the protected functional group does not react under synthetic conditions to which the molecule is subjected. Typical amine protecting groups may include carbamates such as tert-butyloxycarbonyl (Boc), benzyloxy carbamate (CBz), or fluorenylmethyloxycarbonyl (Fmoc) protecting groups.

The phrase “concentrated staining buffer” or “concentrated staining buffer composition”, refers to staining buffer composition produced at, for example, about a 10-fold concentration factor (10×) for dilution, for example, with a diluent such as a biological buffer or water, to provide a working concentration staining buffer composition useful for decreasing non-specific polymer interactions in a multi-color panel when staining a biological sample for flow cytometry analysis. The concentrated staining buffer composition may be manufactured and remain stable in a concentration from 1-fold (1×) to at least 10-fold (10×), or at least 1-fold, 2-fold, 3-fold, 4-fold, 5-fold, 6-fold, 7-fold, 8-fold, 9-fold, or 10-fold more concentrated than the working concentration staining buffer composition. The working concentration staining buffer composition is stable for at least 3 months, 6 months, 9 months, or at least 12 months or more from the date of manufacture when stored in unopened original container at a temperature within a range of from 2 to 40° C., 2 to 30° C., or 2 to 8° C. The concentrated staining buffer composition is stable for at least 3 months, 6 months, 9 months, 12 months, or at least 18 months or more from the date of manufacture when stored in unopened original container at a temperature within a range of from 2 to 40° C., 2 to 30° C., or 2 to 8° C.

The acronym “SN” refers to SuperNova™.

The acronym “SSC” refers to side scatter.

The term “WBC” refers to white blood cells.

The term “quantum yield” (QY) (Φ) or “fluorescence quantum yield” refers to the ratio of the number of photons emitted to the number of photons absorbed. The quantum yield is independent of instrument settings and describes how efficiently a fluorophore converts the excitation energy into fluorescence. Experimentally the relative fluorescence quantum yields can be determined by measuring fluorescence of a fluorophore of known quantum yield with same experimental parameters (excitation wavelength, slit widths, photomultiplier voltage, etc.) as the test dye. The quantum yield may be determined by any method known in the art. For example, the QY may be determined per manufacturer's instructions in a fluorescence spectrofluorometer or fluorescence spectrometer at a selected excitation wavelength. For example, Quantum yield (QY) may be determined on a Shimadzu Rf-6000 Fluorescence Spectrofluorometer by measuring emission intensity at a prespecified wavelength nm from a diluted PBS solution of staining buffer with absorbance at a specific excitation wavelength under specified conditions (e.g., ex slit 1.5, em slit 3.0, 1 cm quartz cuvette). The quantum yield may be calculated, for example, by comparing the intensity measured from the sample and the intensity measured from a standard dye solution under the same experimental conditions. In some embodiments, the QY may be determined, for example, Lawson-Wood et at, Application Note-Fluorescence Spectroscopy, Determination of relative fluorescence quantum yield using the FL5600 fluorescence spectrometer, 2018, PerkinElmer, Inc. The selected excitation wavelength may be, for example, 355 nm. In some embodiments, the QY of a quenched polymer may be compared to parent fluorescent polymer without comprising a quenching moiety.

The term “substrate” as used herein refers to a reagent, medium, surface, substance or material in or on which a molecule is attached or a reaction can take place. The substrate can have a variety of configurations and can be, for example, a solid, fibrous, gel, etc. Substrates, include, but are not limited to, for example, a solid substrate, for example, a solid support such as a particle (e.g., magnetic particle), bead, sheet, a plate with wells, a fibrous mesh, hydrogels, porous matrix, a pin, a microarray surface, a chromatography support.

The term “binding partner” as used herein refers to one of a pair of molecules that have binding specificity for one another (e.g., and antibody and analyte). The binding partner specifically binds with the other molecule to form a binding complex. Any polymer dye or polymer tandem dye described herein can be conjugated to a binding partner at any location convenient on the dye and binding partner. For example, the binding partner may be conjugated to a terminal group on the polymer dye (G¹ or G²) through a functional group.

The term “non-specific interactions” or “non-specific binding” as used herein refers generally to any binding which is not caused by specific binding, and more specifically to the binding of polymer dye conjugates by means other than specific binding of the binding partner to the target analyte. Non-specific binding may result from several factors, including hydrophobicity of the polymers, immune complexing agents, charged proteins, and antibody-interfering proteins which may be present in the staining buffer or biological sample. One type of non-specific binding is the polymer-polymer interactions that may occur between one or more, or two or more fluorescent polymer dye conjugates. Non-specific binding in a test staining buffer composition may be assessed by, for example, comparing FCA dot plots of a mixture of multi-color fluorescent polymer dye conjugates in a biological sample to FCA dot plots of the individual single color fluorescent polymer dye conjugates of the mixture in the same sample, or by determination of R ratios, according to the methods provided herein. For example, if the solution is efficient in preventing non-specific binding polymer-polymer interactions, the respective cell populations will appear well compensated similarly to the staining obtained with the single color conjugates used individually. On the contrary, if the solution is poorly efficient, the populations won't be aligned and will look tilted in flow cytometry analysis dot plots.

An alternative method for measuring the efficiency of the staining buffer compositions for reducing non-specific binding such as polymer-polymer interactions according to the disclosure uses the MFI of the negative and positive populations of the conjugates when they are used individually versus mixed.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)H₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R₂)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms.

The term “functional group” or “substituent” as used herein refers to a group that can be or is substituted onto a molecule or onto an organic group. Examples of substituents or functional groups include, but are not limited to, a halogen (e.g., F, Cl, Br, and 1); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)ₙR, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂—R, N(R)SO₂N(—R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl. Examples of functional groups include, but are not limited to, amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule, or binding partner.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term. “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, benzyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl or benzyl group substituted at any one or more of 2-, 3-, 4-, 5-, or 6-positions of the phenyl ring, benzyl, or a naphthyl group substituted at any one or more of 2- to 8-positions thereof. For example, optionally substituted benzyl groups may be optionally substituted with halogen, hydroxyl, C₁-C₁₂ alkoxy, a PEG group, (OCH₂CH₂)_{f}OCH₃,

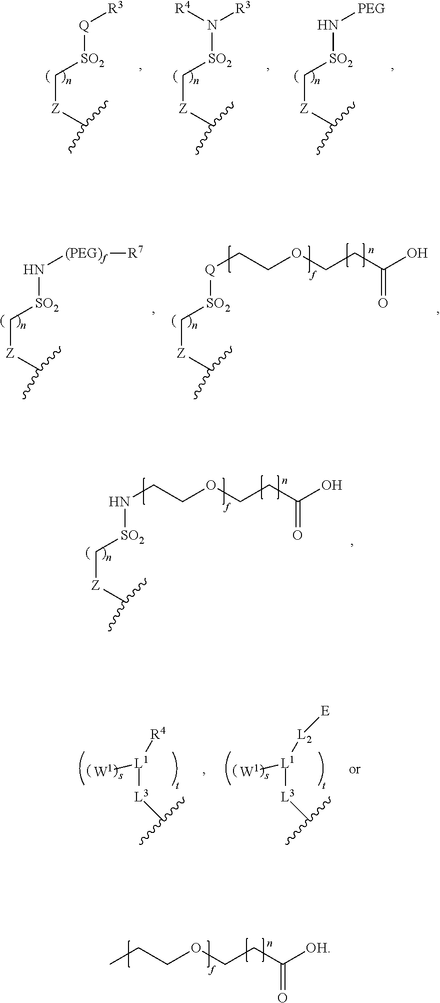

The term “arylene” as used herein refers to cyclic aromatic hydrocarbon groups that do not contain heteroatoms in the ring, and are bivalent groups derived from an aryl group by removal of a hydrogen atom from two ring carbon atoms. In some embodiments, the arylene may be a phenylene, dihydrophenanthrene, fluorene, or binaphthyl group. In some examples, the arylene may be a 9,10-dihydrophenanthrene. In some examples, the arylene is a phenylene In some examples, the arylene is a 1,4-phenylene. In some examples, the arylene is a 1,3-phenylene. In some examples, the heteroarylene may be a carbazole or oxepine. In some examples, the arylene is not a biphenyl group. In some examples, the arylene is not a sulfonyldibenzene group. In some cases, the arylene may be optionally substituted. For example, the optionally substituted arylene may be substituted with halogen, hydroxyl, C₁-C₁₂ alkoxy, a PEG group, (OCH₂CH₂)_{f}OCH₃,

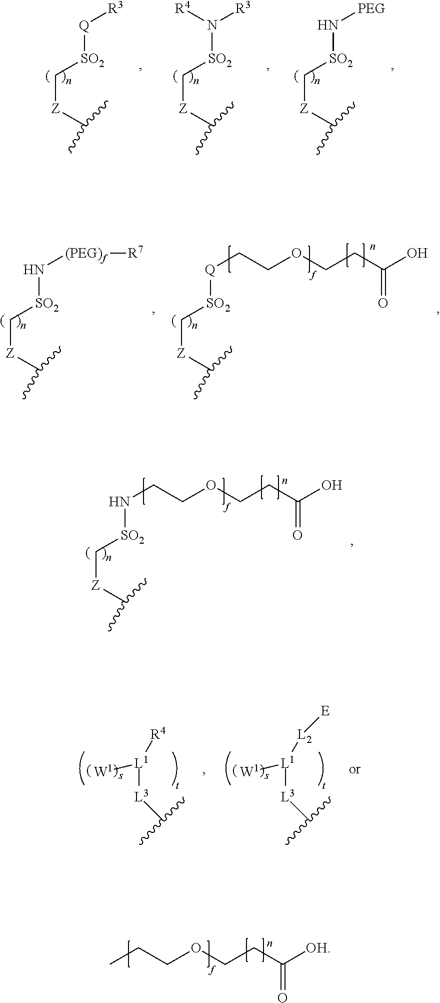

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heteroaryl” as used herein refers to a monocyclic or fused bicyclic or tricyclic aromatic ring assembly containing 5 to 16 ring atoms, where from 1 to 4 of the ring atoms are a heteroatom such as N, O, or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by, for example, alkyl, nitro, or halogen. Pyridyl represents 2-, 3-, or 4-pyridyl, such as 2- or 3-pyridyl. Thienyl represents 2- or 3-thienyl. Quinolinyl represents preferably 2-, 3-, or 4-quinolinyl. Isoquinolinyl represents preferably 1-, 3-, or 4-isoquinolinyl. Benzopyranyl and benzothiopyranyl represents preferably 3-benzopyranyl or 3-benzothiopyranyl, respectively. Thiazolyl represents preferably 2- or 4-thiazolyl, such as 4-thiazolyl. Triazolyl is preferably 1-, 2-, or 5-(1,2,4-triazolyl). Tetrazolyl is preferably 5-tetrazolyl.

Preferably, heteroaryl is pyridyl, indolyl, quinolyl, pyrrolyl, thiazolyl, isoxazolyl, triazolyl, tetrazolyl, pyrazolyl, imidazolyl, thienyl, furanyl, benzothiazolyl, benzofuranyl, isoquinolinyl, benzothienyl, oxazoyl, indazolyl, or any of the radicals substituted, such as mono- or di-substituted.

Substituents for aryl and heteroaryl groups can be selected from -halogen, —OR′, —OC(O)R′, —NR′R″, —SR, —R′, —CN, —NO₂, —CO₂R′, —CONR′R″, —C(O)R′, —OC(O)NR′R″, —NR″C(O)R′, —NR″C(O)₂R′, —NR′—C(O)NR″R″, —NH—C(CH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system, wherein R′, R″, and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂—, or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′—, or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, wherein s and t are independently integers of from 0 to 3, and X is —O—, —NR′—, —S—, —S(O)—, —S(O)₂—, or —S(O)₂NR′—. The substituent R′ in —NR′- and —S(O)₂NR′— is selected from hydrogen or unsubstituted (C₁-C₆)alkyl.

The term “(hetero)arylamino” as used herein refers to an amine radical substituted with an aryl group (e.g., —NH-aryl). An arylamino may also be an aryl radical substituted with an amine group (e.g., -aryl-NH₂). Arylaminos may be substituted or unsubstituted.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R²NH wherein each R is independently selected, such as dialkylamines, diarylamines, aralkylamines, heterocyclylamines and the like; and R³N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺ which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “carbamate” as sued herein refers to a functional group having the structure —NR″CO₂R′, wherein R′ and R″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. Examples of carbamates can include t-Boc, Fmoc, benzyloxy-carboxyl, alloc, methyl carbamate, ethyl carbamate, 9-(2-sulfo)fluorenylmethyl carbamate, 9-(2,7-dibromo)fluorenylmethyl carbamate, Tbfmnoc, Climoc, Bimoc, DBD-Tmoc, Bsmoc, Troc, Teoc, 2-phenylethyl carbamate, Adpoc, 2-chloroethyl carbamate, 1,1-dimethyl-2-haloethyl carbamate, DB-t-BOC, TCBOC, Bpoc, t-Bumeoc, Pyoc, Bnpeoc, N-2-(pivaloylamino)-1,1-dimethylethyl carbamate, and NpSSPeoc.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

As used herein, “oligoether” means an oligomer containing structural repeat units having an ether functionality. As used herein, an “oligomer” means a molecule that contains one or more identifiable structural repeat units or the same or different formula.

As used herein, “sulfonate functional group” or “sulfonate” refers to both the free sulfonate anion (—S(═O)₂O⁻) and salts thereof. Therefore, the term sulfonate encompasses sulfonate salts such as sodium, lithium, potassium, an ammonium sulfonate.

As used herein, the term “sulfonamido” or “sulfonamide” refers to a group of formula —SO₂NHR— or —SO₂N(R⁴)R—, where R can be, but is not limited to, hydrogen, alkyl, aryl, a water-solubilizing moiety, a PEG group, a linker group, a carboxylic group.

Water-solubilizing moieties may be included in the polymer dye to provide for increased water-solubility. While the increase in solubility may vary, in some instances the increase compared to the polymer dye without water-solubilizing moieties may be at least 2 fold or more, e.g., 5 fold, 10 fold, 25 fold, 50 fold, 100 fold or more.

The term “water-solubilizing moiety” refers to a group that is well solvated in aqueous environments e.g., under physiological conditions, and that imparts improved water solubility upon the molecules to which it is attached. The water-solubilizing moiety may be any appropriate hydrophilic group that is well solvated in aqueous environments. In some cases, the hydrophilic water-solubilizing group is charged, e.g., positively or negatively charged. In certain cases, the hydrophilic water-solubilizing group is a neutral hydrophilic group. In some embodiments, the water-solubilizing moiety is a hydrophilic polymer, e.g., a polyethylene glycol, a cellulose, a chitosan, or a derivative thereof. Water-solubilizing moieties may include, but are not limited to, carboxylate, phosphonate, phosphate, sulfonate, sulfate, sulfinate, sulfonium, ester, sulfonamide, polyethylene glycols (PEGs), modified PEGs, hydroxyl, amine, ammonium, guanidinium, pyridinium, polyamine and sulfonium, polyalcohols, straight chain or cyclic saccharides, primary, secondary, tertiary, or quaternary amines and polyamines, phosphonate groups, phosphinate groups, ascorbate groups, glycols. In some embodiments, the water-solubilizing moiety is a PEG group.

The term “water-soluble UV-absorbing polymer dye” refers to a UV-absorbing polymer dye, tandem dye, or quenched dye, or conjugate thereof, that exhibits solubility in water at room temperature in excess of 1 mg/mL, 5 mg/mL, 10 mg/mL, 20 mg/mL, 30 mg/mL, 40 mg/mL, or 50 mg/mL, or from 1-250 mg/mL, 2-200 mg/mL, 3-150 mg/mL, 4-125 mg/mL, 5-100 mg/mL, 7-70 mg/mL, or 10-50 mg/mL.

As used herein, the term “polyethylene glycol,” or “poly(ethylene glycol)”, or “PEG” refers to the family of biocompatible water-solubilizing linear polymers based on the ethylene glycol monomer unit described by the formula —(CH₂—CH₂—O⁻)ₙ— or a derivative thereof. A PEGn moiety may be employed as a water-solubilizing moiety. The water-solubilizing moieties may be capable of imparting solubility in water at room temperature to the molecule to which it is attached of at least 1 mg/mL, at least 5 mg/mL, at least 10 mg/mL, at least 20 mg/mL, at least 30 mg/mL, at least 40 mg/mL, or at least 50 mg/mL, or from 1-250 mg/mL, 2-200 mg/mL, 3-150 mg/mL, 4-125 mg/mL, 5-100 mg/mL, 7-70 mg/mL, or 10-50 mg/mL. In some embodiments, “n” is 1000 or less, 500 or less, 200 or less, 100 or less, 50 or less, 40 or less, 30 or less, 20 or less, 15 or less, such as 3 to 15, or 10 to 15. It is understood that the PEG polymeric group may be of any convenient length and may include a variety of terminal groups and/or further substituent groups, including but not limited to, alkyl, aryl, hydroxyl, alkoxy, alkanol, —OCH₃, —O—C₁-C₄alkyl, amino, acyl, carboxylic acid, carboxylate ester, acyloxy, and amido terminal and/or substituent groups. The number after “PEG” refers to the average molecular weight.

The term “Mw” refers to weight average molecular weight, and “Mn” refers to number average molecular weight. The average molecular weight of polymers may be determined by any appropriate method, for example, average molecular weight of polymers maybe determined by light scattering techniques or size exclusion chromatography. Gel permeation chromatography may be used to determine the number average molecular weight, weight average molecular weight of the polymers.

The term “cross talk index” refers to the UV-polymer dyes percentage of residual absorbance at 405 nm relative to absorbance at 355 nm, excitation wavelengths of violet and UV laser, respectively. Calculation of cross-talk index from absorption spectra may be performed by obtaining absorption spectra of a filtered (0.22 u) solution of the polymer dye in PBS. Absorbance at wavelengths 355 un and 405 nm are recorded. Ratio of absorbance at 405 nm to 355 nm is measured which gives a fair idea about overall leakage that can be expected in the pacific blue channel for a conjugate made from this polymer upon excitation with a violet laser.

As used herein, the term “carboxylate” refers to the conjugate base of a carboxylic acid, which generally can be represented by the formula RCOO. For example, the term “magnesium carboxylate” refers to the magnesium salt of the carboxylic acid.

As used herein, the term “activated ester” refers to carboxyl-activating groups employed in peptide chemistry to promote facile condensation of a carboxyl group with a free amino group of an amino acid derivative. Descriptions of these carboxyl-activating groups can be found in general textbooks of peptide chemistry; for example, K. D. Kopple, “Peptides and Amino Acids”, W. A. Benjamin, Inc., New York, 1966, pp. 50-51 and E. Schroder and K. Lubke, “The Peptides”; Vol. 1, Academic Press, New York, 1965, pp. 77-128.

The terms “hydrazine” and “hydrazide” refer to compounds that contain singly bonded nitrogens, one of which is a primary amine functional group.

The term “aldehyde” as used herein refers to a chemical compound that has a —CHO group.

The term “thiol” as used herein refers to a compound that contains a functional group composed of a sulfur-hydrogen bond. The general chemical structure of the thiol functional group is R—SH, where R represents an alkyl, alkene, aryl, or other carbon-containing group of atoms.

The term “silyl” as used herein refers to Si(R^{z})₃ wherein each R^{z} independently is alkyl, aryl, or other carbon-containing group of atoms.

The term “diazonium salt” as used herein refers to a group of organic compounds with a structure of R—N₂⁺X⁻, wherein R can be any organic residue (e.g., alkyl or aryl) and X is an inorganic or organic anion (e.g., halogen).

The term “triflate”, as referred to as “trifluromethanesulfonate”, is a group with the formula CF₃SO₃.

The term “boronic acid” as used herein refers to a structure —B(OH)₂. It is recognized by those skilled in the art that a boronic acid may be present as a boronate ester at various stages in the synthesis. Boronic acid is meant to include such esters. The term “boronic ester” or “boronate ester” as used herein refers to a chemical compound containing a —B(Z¹)(Z²) moiety, wherein Z¹ and Z² together form a moiety where the atom attached to boron in each case is an oxygen atom. The boronic ester moiety can be a 5-membered ring, a 6-membered ring, or a mixture of a 5-membered ring and a 6-membered ring.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group. A hydrocarbylene group is a diradical hydrocarbon, e.g., a hydrocarbon that is bonded at two locations.

### UV-Absorbing Polymers.

In various aspects the present disclosure provides a UV-absorbing polymer having the structure of Formula I:

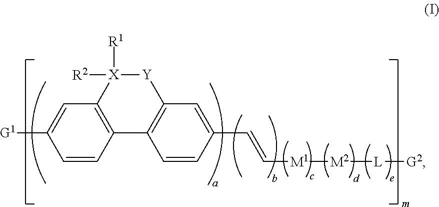

wherein each X is independently selected from the group consisting of C and Si; each Y is independently selected from the group consisting of a bond, CR¹R², CHR¹, CHR², SiHR², SiHR¹, and SiR¹R², and when Y is a bond X is directly bonded to both rings; each R¹ is independently selected from the group consisting of a water-solubilizing moiety, alkyl, alkene, alkyne, cycloalkyl, haloalkyl, (hetero)aryloxy, (hetero)arylamino, aryl, heteroaryl, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide,

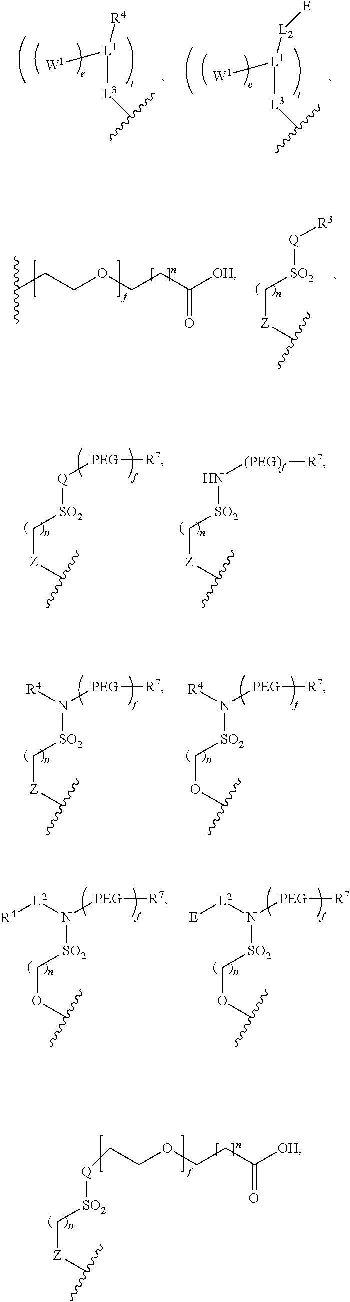

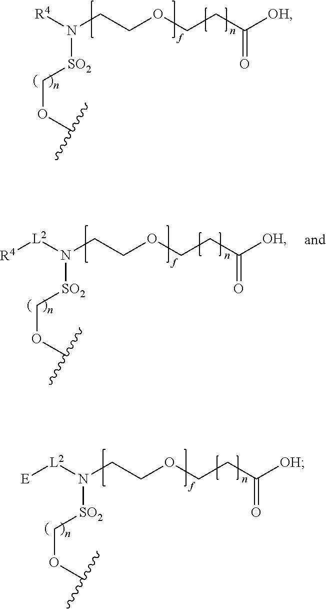

each R² is independently selected from the group consisting of a water-solubilizing moiety, a linker moiety, H, alkyl, alkene, alkyne, cycloalkyl, haloalkyl, alkoxy, (hetero)aryloxy, aryl, heteroaryl, (hetero)arylamino, a PEG group, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

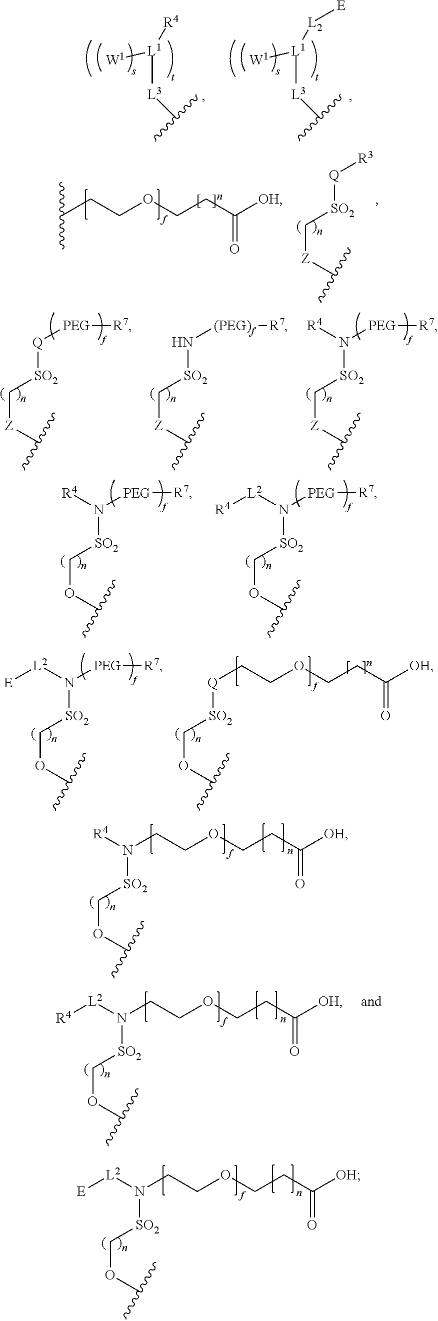

each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, and a PEG group; each Z is independently selected from the group consisting of CH₂, CHR⁴, O, NH, and NR⁴; each Q is independently selected from the group consisting of a bond, NH, NR⁴, C₁-C₁₂ alkylene, CHR⁴, and CH₂; each R⁴ is independently selected the group consisting of H, a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OCH₃ wherein each x′ is independently an integer from 0-20 and each y′ is independently an integer from 0-50, Z—(CH₂)ₙ—SO₂-Q-R³, a C₂-C₁₈ (hetero)aryl group, amide, amine, carbamate, carboxylic acid, carboxylate ester, maleimide, activated ester, N-hydroxysuccinimidyl, hydrazine, hydrazone, azide, aldehyde, thiol, and protected groups thereof; each W¹ is independently a water-solubilizing moiety;
L¹, L², and L³ are each independently selected linker moieties; each E is independently selected from the group consisting of a chromophore, a functional moiety, a substrate, and a binding partner; each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester, and C₁-C₁₂ alkoxy; at least one of R¹, R³, or R⁴ comprises a water-solubilizing moiety;
each M¹ is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted; each M² is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than MU, and wherein M² and M¹ are evenly or randomly distributed along the polymer main chain; each optional linker L is an aryl or heteroaryl group evenly or randomly distributed along the polymer main chain and that is substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule, or binding partner; G¹ and G² are each independently selected from the group consisting of an unmodified polymer terminus and a modified polymer terminus, optionally conjugated to E; a, c, d, and e define the mol % of each unit within the structure Which each can be evenly or randomly repeated along the polymer main chain and where a is a mol % from 10 to 100%, c is a mol % from >0 to 90%, each d is a mol % from 0 to 90%, and each e is a mol % from 0 to 25%; each b is independently 0 or 1; each f is independently an integer from 0 to 50; m is an integer from 1 to about 10,000; each n is independently an integer from 1 to 20; s is 1 or 2; and t is 0, 1, 2, or 3.

In the UV-absorbing polymer dye according to Formula (I), each X can be independently selected from C and Si. Each Y can be independently selected from a bond, CR¹R², CHR², CHR¹, SiHR², SiHR¹ and SiR¹R², and when Y is a bond X is directly bonded to both rings. Each R¹ can be independently selected from polyethylene glycol (PEG), ammonium alkyl salt, ammonium alkyloxy salt, ammonium oligoether salt, sulfonate alkyl salt, sulfonate alkoxy salt, sulfonamido oligoether, and —Z—(CH₂)ₙ—SO₂-Q-R³. In some embodiments, Y is a bond and R¹ and R² are each independently —Z—(CH₂)ₙ—SO₂-Q-R³. Each R² can be independently selected from H, alkyl, alkene, alkyne, cycloalkyl, haloalkyl, alkoxy, (hetero)aryloxy, aryl, (hetero)arylamino, a PEG group, ammonium alkyl salt, ammonium alkyloxy salt, ammonium oligoether salt, sulfonate alkyl salt, sulfonate alkoxy salt, sulfonate oligoether salt, sulfonamido oligoether, and —Z—(CH₂)ₙ—SO₂-Q-R³. Each R³ can be independently selected from H, alkyl, alkene, alkyne, cycloalkyl, haloalkyl, alkoxy, (hetero)aryloxy, aryl, (hetero)arylamino, and a PEG group (e.g., -PEG-R⁵, or -PEG-OMe). Each Z can be independently selected from C, O, and N. Each Q can be independently selected from a bond, NH, NR, C₁-C₁₂ alkylene, and CH₂. Each R³ can be independently selected from a chromophore (e.g., an acceptor dye), halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OCH₃ wherein each x′ is independently an integer from 0-20 and each y′ is independently an integer from 0-50, —Z—(CH₂)ₙ—SO₂-Q-R³, and a C₂-C₁₈ (hetero)aryl group. Each modifying unit M¹ and M² can be independently selected from an arylene or heteroarylene capable of altering the band gap of the polymer. Each M¹ can be independently selected from an R⁴- and/or trifluoromethyl-substituted arylene that is optionally further substituted, an R⁴- and/or trifluoromethyl-substituted heteroarylene that is optionally further substituted, an R⁴ and/or trifluoromethyl-substituted 9,10-dihydrophenanthrene that is optionally further substituted, and a binaphthyl that is optionally substituted. For example, each M¹ can have one to four (e.g., 1, 2, 3, or 4) R⁴ or trifluoromethyl substituents. Each M² can be independently selected from an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted wherein M² has a different structure than M¹. For example, each M² can have one to four (e.g., 1, 2, 3, or 4) R or trifluoromethyl substituents. Each linker L can be an aryl or heteroaryl group evenly or randomly distributed along the polymer main chain and that is substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule or binding partner.

In some examples, the variables G¹ and G² may each independently be selected from hydrogen, halogen, alkyne, halogen substituted aryl, silyl, diazonium salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), or optionally substituted fluorene, wherein the optionally substituted aryl, heteroaryl, fluorene, or DHP maybe substituted with one or more pendant chains terminated with a functional group, for example, selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof, for conjugation to a substrate or binding partner.

The variables a, c, d, and e define the mol % of each unit within the structure which each can be evenly or randomly repeated and where a is a mol % from 10 to 100%, c is a mol % from >0 to 90%, each d is a mol % from 0 to 90%, and each e is a mol % from 0 to 25%. Each b can be independently 0 or 1. The variable m can be an integer from 1 to about 10,000 (e.g., at least 2, or less than 10,000 but greater than, less than, or equal to 5, 10, 15, 20, 25, 50, 100, 150, 200, 250, 500, 1,000, 5,000, or 7,500). Each n can independently be an integer from 1 to 20 (e.g., 1, 2, 3, 4, 5, 6, 7, 8, 9, 10, 11, 12, 13, 14, 15, 16, 17, 18, 19, or 20). The UV-absorbing polymer can be water-soluble.

In the polymers described herein, the units defined by a, c, d, and e can occur within any order in the polymer backbone, such as the order shown in the Formula (I) structure, or such as in a different order. The units can occur in an even or random arrangement within the polymer backbone.

The term “polymer dye” as used herein may refer to a UV-absorbing polymer dye, UV-absorbing polymer dye conjugate, UV-absorbing polymer tandem dye, UV-absorbing polymer tandem dye conjugate, or quenched UV-absorbing polymer dye. The polymer dye may be a tandem polymer dye comprising one or more acceptor dye moieties attached to the backbone, for example, through a linker L, or to the monomer, for example, at R¹ or R², that will provide for monitoring the emission of the acceptor dyes attached to the backbone through energy transfer. In some embodiments, at least one of R¹ or R² is —Z—(CH₂), —SO₂—N(chromophore)-R³, —Z—(CH₂)ₙ—SO₂—N(L²-chromophore)-R³,

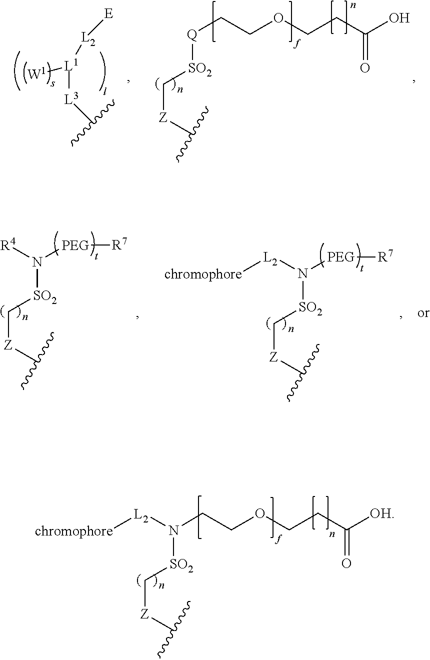

The chromophore can be an acceptor dye that allows excitation of the polymer backbone and allow monitoring of the emission of the acceptor dyes attached to the backbone.

Acceptor dyes useful in the tandem polymer dyes may include, for example, a cyanine dye, a xanthene dye, a coumarin dye, a thiazine dye, an acridine dye, FITC, CY3B, Cy55, Alexa 488, Alexa750, Texas red, Cy3B, Cy3.5, Cy5, Cy7, Cy55, Alexa 750, 800 CW, Biotium (CF 555, diethyl coumarin, DY705 (Dyomics), DY431, DY485XL, DY500XL, DY610, DY640, DY654, DY 682, DY 700, DY 701, DY 704, DY 730, DY 731, DY732, DY 734, DY 752, DY 778, DY 782, DY 800, DY 831. The acceptor dye may be a pendant acceptor dye.

For example, acceptor dyes useful in the tandem polymer dyes include, for example, Dyomics DY 704, FITC, CY3B, Cy55, Alexa 488, Texas red, Cy5, Cy7, Alexa 750, and 800CW. The tandem dye may be a UV polymer according to the disclosure comprising one or more, two or more, three or more, 1-30, 2-20, or 2.5-10 acceptor dye moieties.

In some embodiments, the acceptor dye moiety may be or derived from, for example, Dyomics DY 704.

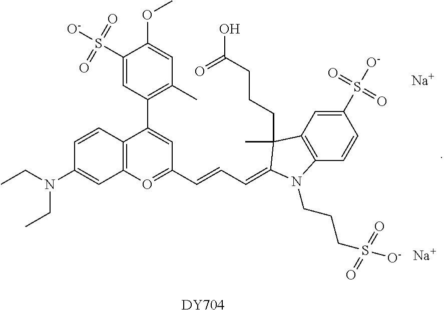

In some embodiments, the UV polymer dye can be a quenched UV polymer comprising one or more quenching moieties attached to the backbone, for example, through a linker L, or to the monomer, for example, at R¹ or R². In some embodiments, at least one R² is —Z—(CH₂)ₙ—SO₂—N(quenching moiety)-R³. In some embodiments, the acceptor dye can be a quenching moiety. For example, the quenching moeity may be selected from, for example DABCYL DABSYL, Black Hole Quencer1 (BHQ1), BHQ-0, Deep Dark Quencher I, DDQI, EDQ, QSY7, QSY9, QSY35, TAMRA (carboxytetramethylrhodamine), Dabcyl Q, Dabcyl plus, Anaspec 490Q, Dyomics 425Q, Dyomics 505Q. Non-limiting examples of quenching moieties may include, for example:

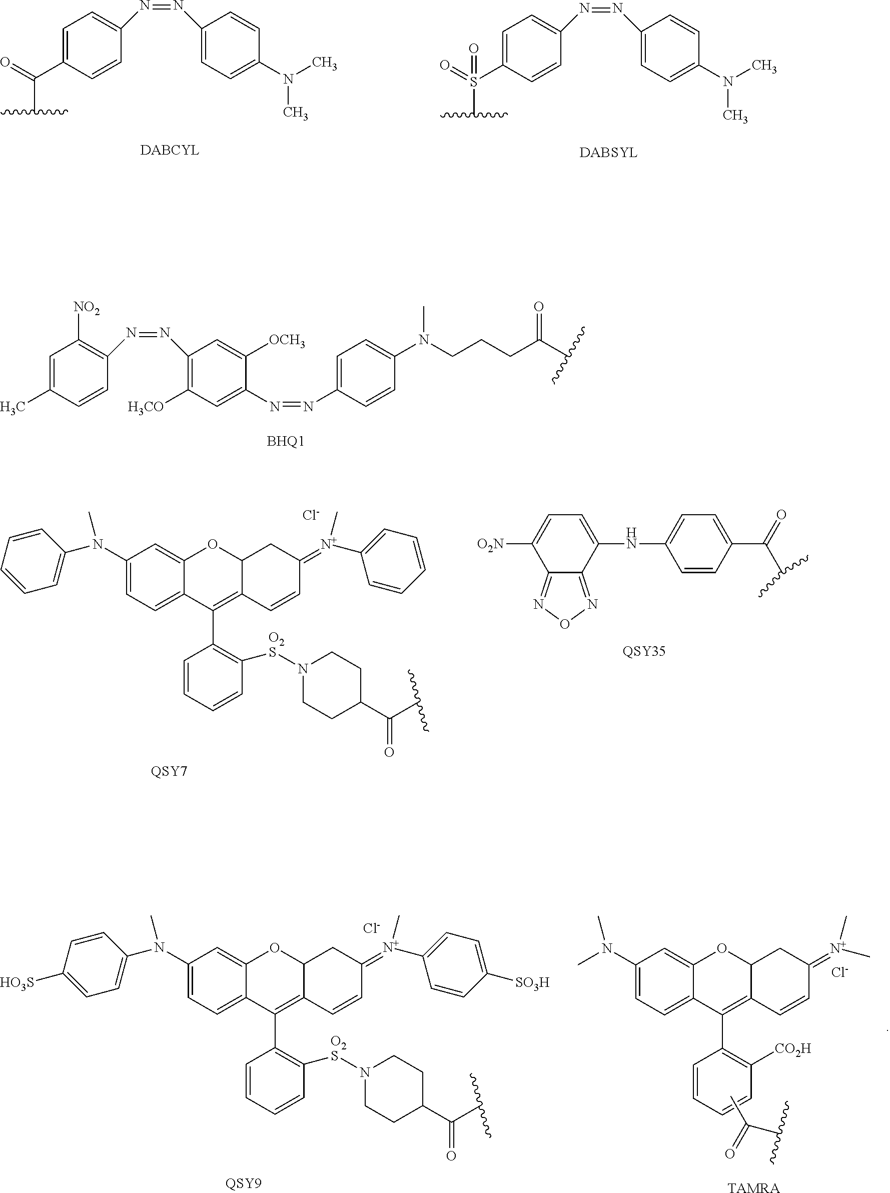

In some embodiments, a quenched JV polymer dye is provided according to the present disclosure comprising 1-30, 2-20, or 2.5-10 quenching moieties. In some embodiments, the quenching moiety is a dabcyl moiety. In some embodiments, the quenched LUV polymer dye comprises 2.5-10 dabcyl moieties (Poly-Dabcyl LV Polymer). As provided herein, the tandem dye or quenched polymer dye may be prepared by reacting an active ester such as an NHS ester of the acceptor moiety, for example, NHS ester moiety of the quenching moiety, with the UV polymer dye of the disclosure. Such acceptor dye NHS esters are commercially available, for example, DY-705 NHS ester from Dyomics, or Dabcyl SE (Dabcyl succinimidyl ester) from Abcam.

A staining buffer composition is provided comprising a quenched UV polymer dye according to the disclosure comprising at least one quenching moiety, optionally 1-30, 2-20, or 2.5-10 quenching moieties. The quenching moieties may be selected from any appropriate quenching moiety; non-limiting examples may include DABCYL, DABSYL, BHQ1, BHQ0, DDQI, EDQ, QSY7, QSY9, QSY35, TAMRA Dabcyl Q, Dabcyl plus, 490Q, 425Q, and 505Q.

The polymer can have the structure of Formula II:

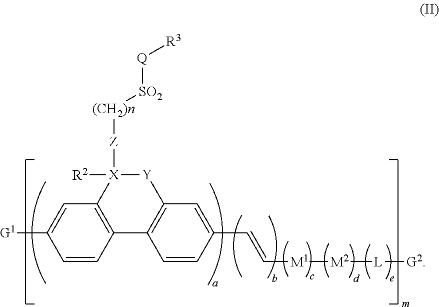

The polymer can have the structure of Formula III:

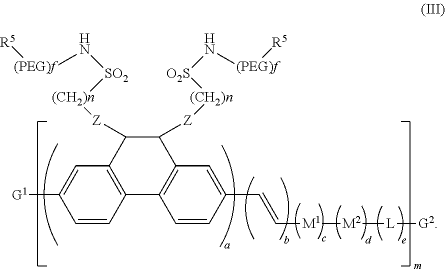

Each f can independently be an integer from 0 to 50. Each R⁵ can be independently selected from the group consisting of H, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, and C₁-C₁₂ alkoxy.

The polymer can have the structure of Formula IV:

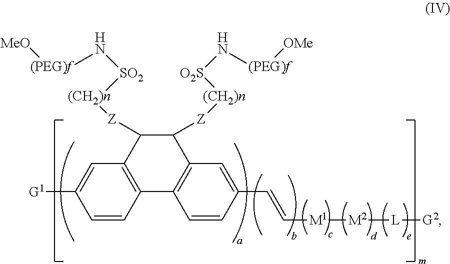

wherein each f can independently be an integer from 0 to 50, 10 to 20, or 11 to 18.

The polymer can be a copolymer having the structure of Formula V:

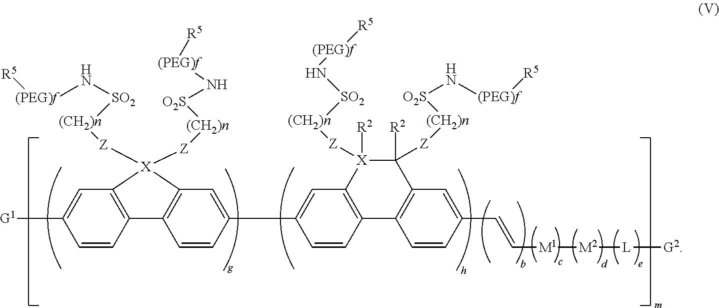

The variables g and h together can be a mol % from 10 to 100%. Each f can independently be an integer from 0 to 50. Each R⁵ can be independently selected from the group consisting of H, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, and C₁-C₁₂ alkoxy.

The polymer can have the structure of Formula VI:

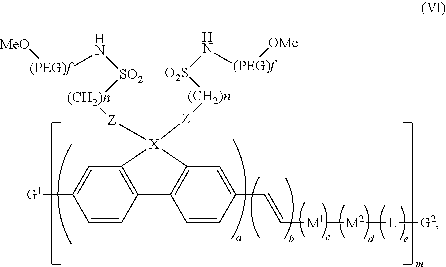

wherein each f can independently be an integer from 0 to 50. Each R⁵ can be independently selected from the group consisting of H, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, and C₁-C₁₂ alkoxy.

The polymer can be a copolymer having the structure of Formula VII:

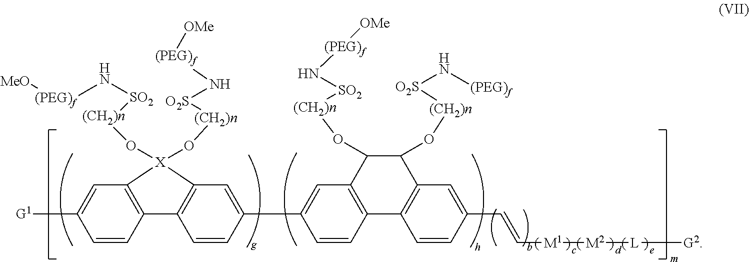

The variables g and h together can be a mol % from 10 to 100%. Each f can independently be an integer from 0 to 50.

The polymer can have the structure of Formula VIII:

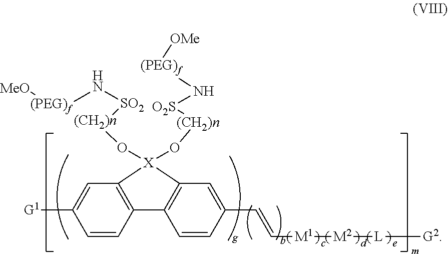

Each f can independently be an integer from 0 to 50.

In the Formulas described herein, each f (i.e., the multiplicity of the PEG group) can independently be an integer from 0 to 50, such as 5 to 40, 3 to 30, 5 to 20, 10 to 25, 10 to 20, 11 to 18, or less than 50 but greater than or equal to 0, 1, 2, 3, 4, 5, 6, 7, 8, 9, 10, 11, 12, 13, 14, 15, 16, 17, 18, 19, 20, 22, 24, 26, 28, 30, 35, 40, or 45.

In the Formulas described herein, modifying unit M¹ can be capable of altering the band gap of the polymer. Each M¹ can be independently selected from an R⁴- and/or trifluoromethyl-substituted arylene that is optionally further substituted and an R⁴- and/or trifluoromethyl-substituted heteroarylene that is optionally further substituted. Each M¹ can independently be a halide-, MeO-PEG-CH₂—, and/or MeO-PEG-substituted arylene (e.g., phenylene) that is optionally further substituted. Each M can be independently selected from a halide- (e.g., fluorine-) and/or trifluoromethyl-substituted arylene that is optionally further substituted and a halide- (e.g., fluorine-) and/or trifluoromethyl-substituted heteroarylene that is optionally further substituted. Each M¹ can independently be a halide-substituted arylene having 1-4 halide substituents. Each M¹ can independently be a fluorine-substituted arylene having 1-4 fluorine substituents. Each M¹ can independently be a halide-substituted phenylene having 1-4 halide substituents, wherein the phenylene is optionally further substituted. Each M¹ can independently be a fluorine-substituted phenylene having 1-4 fluorine substituents, wherein the phenylene is optionally further substituted. Each M¹ can independently be a halide-substituted phenylene having 2 or 3 halide substituents, or a fluorine-substituted phenylene having 2 or 3 fluorine substituents.

Each M¹ can be a dihalide-substituted phenylene. Each M¹ can independently be selected from: a phenylene having the 1- and 4-positions thereof substituted into the backbone of the polymer and that is dihalide-substituted with halide at the 2- and 3-positions, the 2- and 5-positions, or at the 2- and 6-positions; a phenylene having the 1- and 4-positions thereof substituted into the backbone of the polymer and that is trihalide-substituted with halide at the 2-, 3-, and 5-positions; a phenylene having the 1- and 3-positions thereof substituted into the backbone of the polymer and that is dihalide-substituted with halide at the 2- and 4-positions, the 2- and 5-positions, the 4- and 5-positions, or the 4- and 6-positions; and a phenylene having the 1- and 3-positions thereof substituted into the backbone of the polymer and that is trihalide-substituted with halide at the 4-, 5-, and 6-positions, at the 2-, 4-, and 5-positions, or at the 2-, 4-, and 6-positions. Each M¹ can be independently selected from: a phenylene having the 1- and 4-positions thereof substituted into the backbone of the polymer and that is dihalide-substituted with halide at the 2- and 3-positions, the 2- and 5-positions, or at the 2- and 6-positions; and a phenylene having the 1- and 3-positions thereof substituted into the backbone of the polymer and that is dihalide-substituted with halide at the 2- and 4-positions, the 2- and 5-positions, the 4- and 5-positions, or the 4- and 6-positions.

Each M¹ can be a difluoro-substituted phenylene. Each M¹ can independently be selected from: a phenylene having the 1- and 4-positions thereof substituted into the backbone of the polymer and that is dihalo- (e.g. difluoro-) substituted with fluorine at the 2- and 3-positions, the 2- and 5-positions, or at the 2- and 6-positions; a phenylene having the 1- and 4-positions thereof substituted into the backbone of the polymer and that is trihalo- (e.g. trifluoro-) substituted with fluorine at the 2-, 3-, and 5-positions; a phenylene having the 1- and 3-positions thereof substituted into the backbone of the polymer and that is dihalo- (e.g. difluoro-) substituted with fluorine at the 2- and 4-positions, the 2- and 5-positions, the 4- and 5-positions, or the 4- and 6-positions; and a phenylene having the 1- and 3-positions thereof substituted into the backbone of the polymer and that is trihalo- (e.g. trifluoro-) substituted with fluorine at the 4-, 5-, and 6-positions, at the 2-, 4-, and 5-positions, or at the 2-, 4-, and 6-positions. Each M¹ can be independently selected from: a phenylene having the 1- and 4-positions thereof substituted into the backbone of the polymer and that is difluoro-substituted with fluorine at the 2- and 3-positions, the 2- and 5-positions, or at the 2- and 6-positions; and a phenylene having the 1- and 3-positions thereof substituted into the backbone of the polymer and that is difluoro-substituted with fluorine at the 2- and 4-positions, the 2- and 5-positions, the 4- and 5-positions, or the 4- and 6-positions.

In some embodiments, each R⁴ may be independently selected from F, Cl, —CF₃, —OCH₃, —CN, —CH₃, —O(CH₂CH₂O)_{f}OCH₃, and —CO₂H.

Each M¹ can be independently selected from:

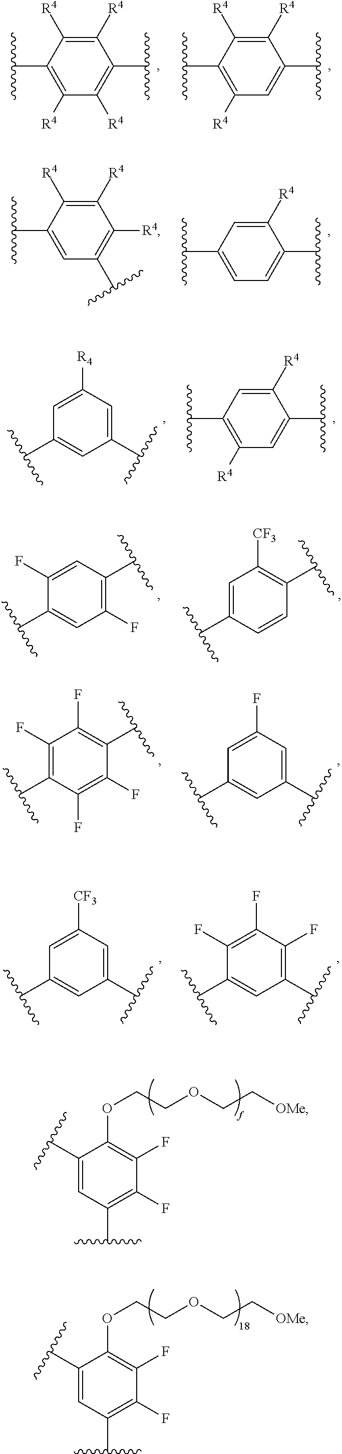

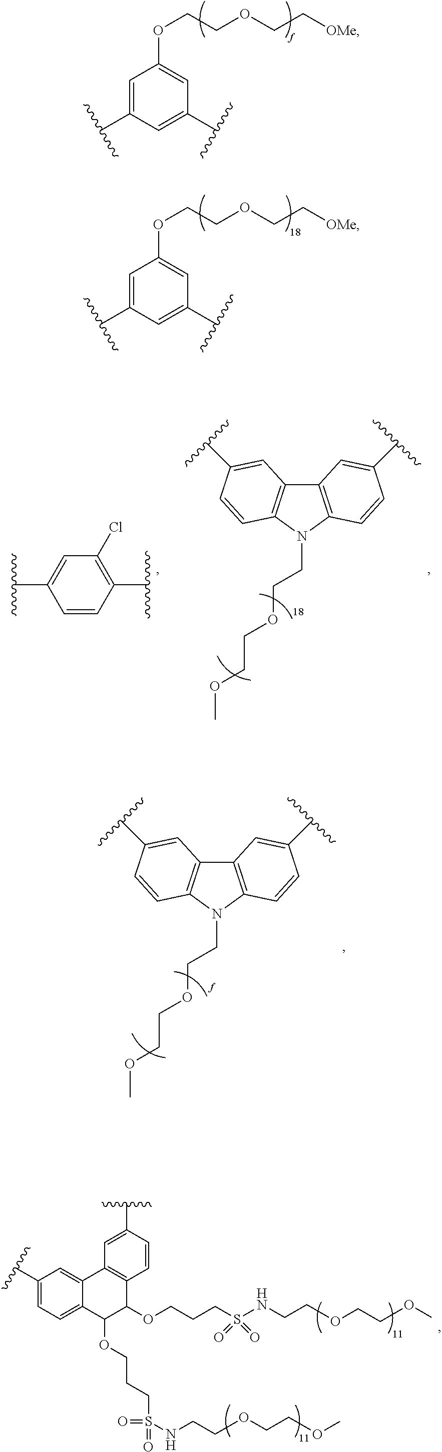

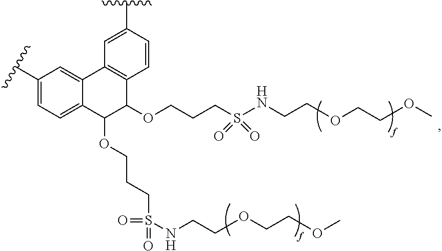

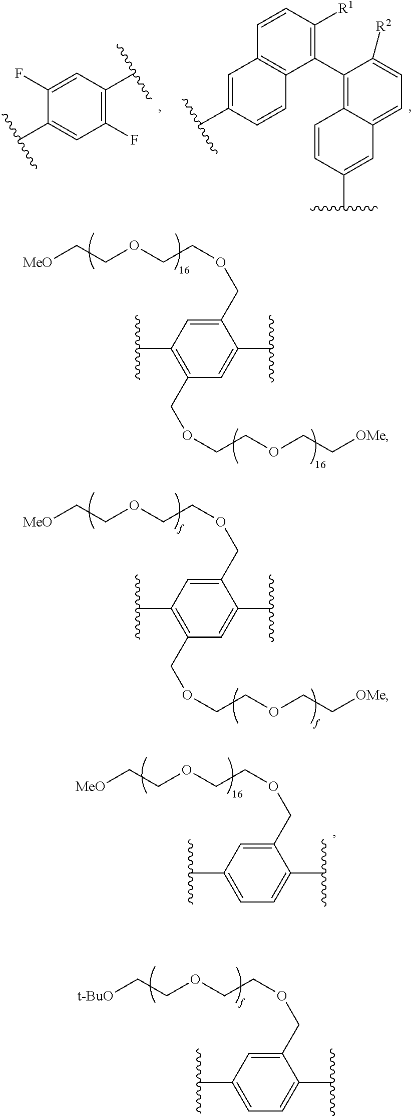

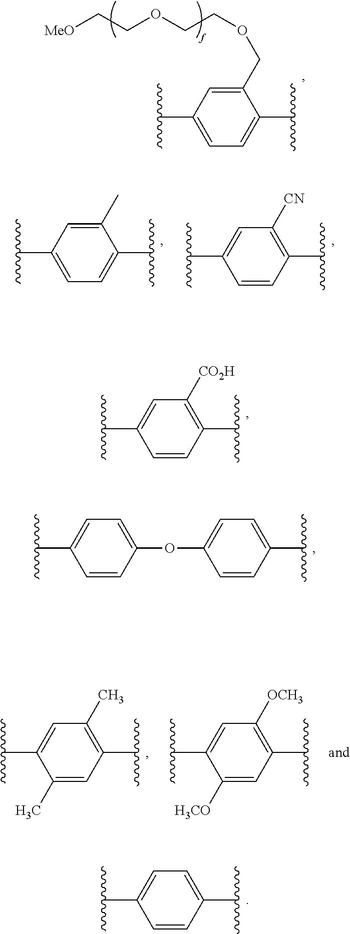

Each M¹ can be independently selected from:

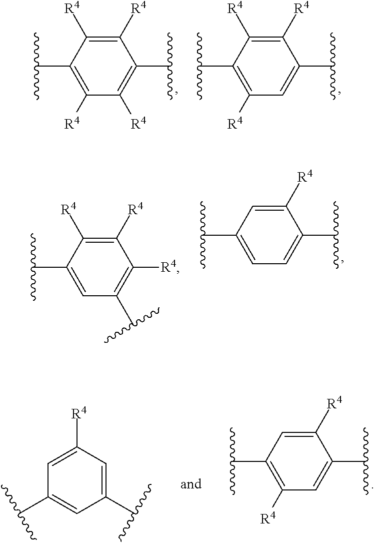

Each M¹ can be independently selected from:

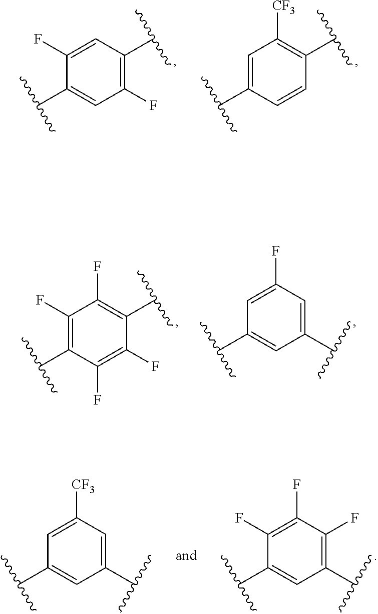 [SMILES: *c1cc(F)c(*)cc1F ; *c1ccc(*)c(C(F)(F)F)c1 ; *c1c(F)c(F)c(*)c(F)c1F ; *c1cc(*)cc(F)c1 ; *c1cc(*)cc(C(F)(F)F)c1 ; *c1cc(*)c(F)c(F)c1F]

Each M¹ can be a phenylene having the 1- and 4-positions thereof substituted into the backbone of the polymer and that is 2,5-difluoro substituted. Each M¹ can be:

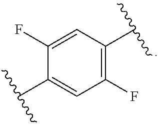 [SMILES: *c1cc(F)c(*)cc1F]

Each M¹ can be a binaphthyl that is optionally substituted. Each M¹ can be:

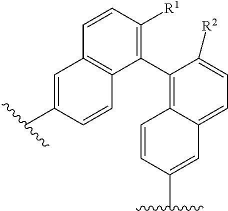

In the Formulas described herein, modifying unit M² can be capable of altering the band gap of the polymer.

M² can have a different structure than M¹.

Each M² can be independently selected from:

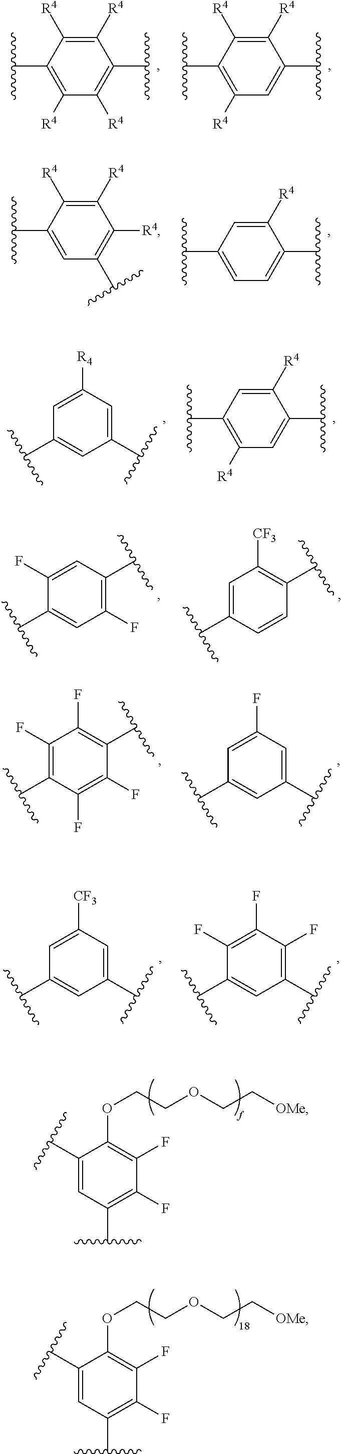

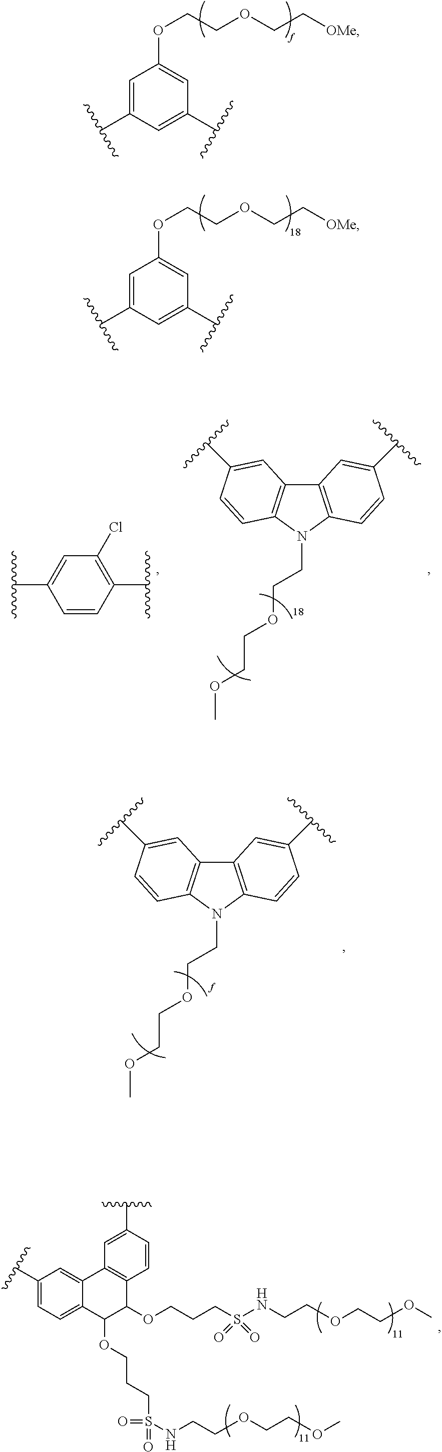

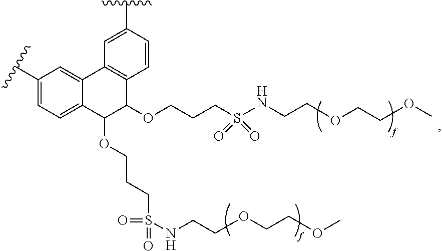

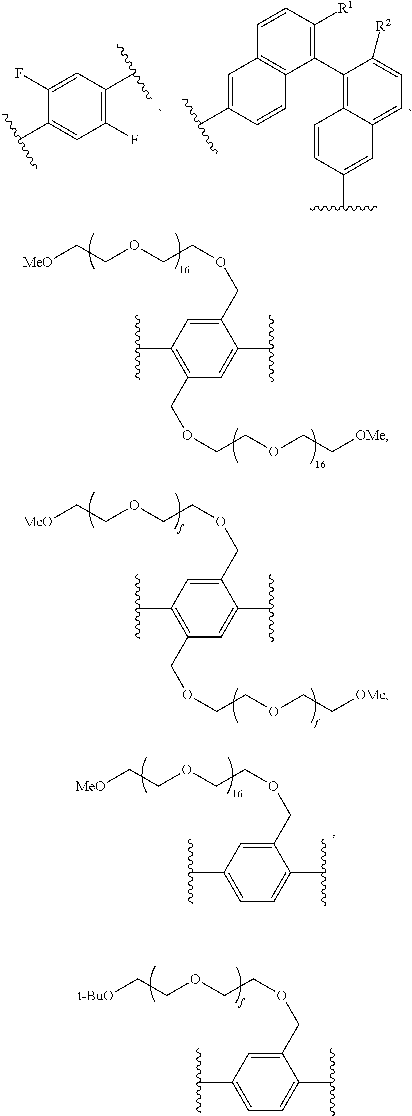

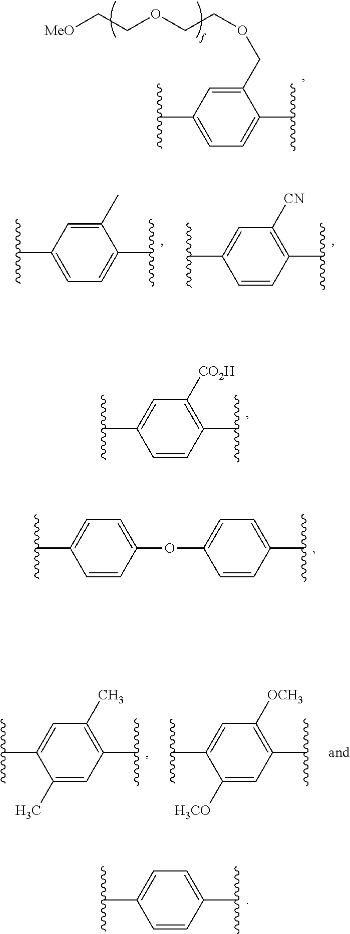

Each M² can be independently selected from:

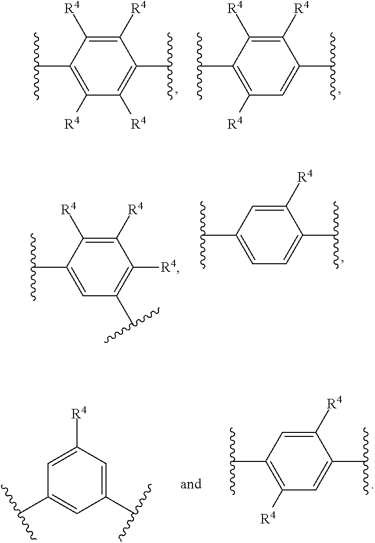

Each M² can be independently selected from:

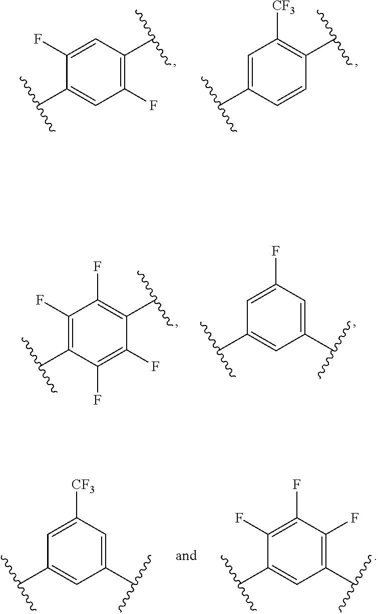 [SMILES: *c1cc(F)c(*)cc1F ; *c1ccc(*)c(C(F)(F)F)c1 ; *c1c(F)c(F)c(*)c(F)c1F ; *c1cc(*)cc(F)c1 ; *c1cc(*)cc(C(F)(F)F)c1 ; *c1cc(*)c(F)c(F)c1F]

Each M² can be independently selected from an R⁴- and/or trifluoromethyl-substituted arylene that is optionally further substituted and an R⁴- and/or trifluoromethyl-substituted heteroarylene that is optionally further substituted. Each M² can independently be a halide-, MeO-PEG-CH₂—, and/or MeO-PEG-substituted arylene (e.g., phenylene) that is optionally further substituted. Each M² can be independently selected from a fluorine- and/or trifluoromethyl-substituted arylene that is optionally further substituted and a fluorine- and/or trifluoromethyl-substituted heteroarylene that is optionally further substituted. Each M² can independently be a halogen-substituted arylene having 1-4 halide substituents. Each M² can independently be a fluorine-substituted arylene having 1-4 fluorine substituents. Each M² can independently be a halide-substituted phenylene having 1-4 halide substituents, wherein the phenylene is optionally further substituted. Each M² can independently be a fluorine-substituted phenylene having 1-4 fluorine substituents, wherein the phenylene is optionally further substituted. Each M² can independently be a halide-substituted phenylene having 2 or 3 halide substituents, or a fluorine-substituted phenylene having 2 or 3 fluorine substituents.

Each M² can be independently selected from:

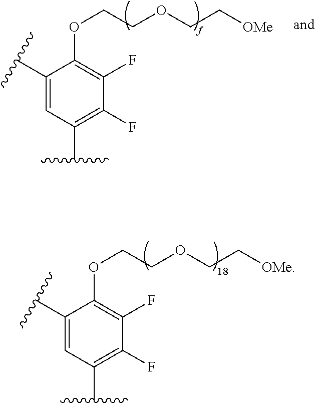

Each M² can be a trihalide-substituted phenylene. Each M² can be independently selected from: a phenylene having the 1- and 4-positions thereof substituted into the backbone of the polymer and that is trihalide-substituted with halide at the 2-, 3-, and 5-positions; and a phenylene having the 1- and 3-positions thereof substituted into the backbone of the polymer and that is trihalide-substituted with halide at the 4-, 5-, and 6-positions, at the 2-, 4-, and 5-positions, or at the 2-, 4-, and 6-positions. Each M² can be a phenylene having the 1- and 3-positions thereof substituted into the backbone of the polymer and that is 4,5,6-trihalide substituted.

Each M² can be a trifluoro-substituted phenylene. Each M² can be independently selected from: a phenylene having the 1- and 4-positions thereof substituted into the backbone of the polymer and that is trifluoro-substituted with fluorine at the 2-, 3-, and 5-positions; and a phenylene having the 1- and 3-positions thereof substituted into the backbone of the polymer and that is trifluoro-substituted with fluorine at the 4-, 5-, and 6-positions, at the 2-, 4-, and 5-positions, or at the 2-, 4-, and 6-positions. Each M² can be a phenylene having the 1- and 3-positions thereof substituted into the backbone of the polymer and that is 4,5,6-trifluoro substituted. Each M² can be:

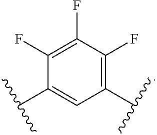 [SMILES: *c1cc(*)c(F)c(F)c1F]

Each M² can be a binaphthyl that is optionally substituted. Each M² can be:

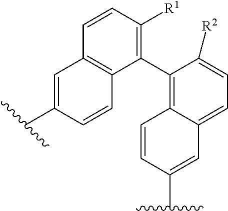

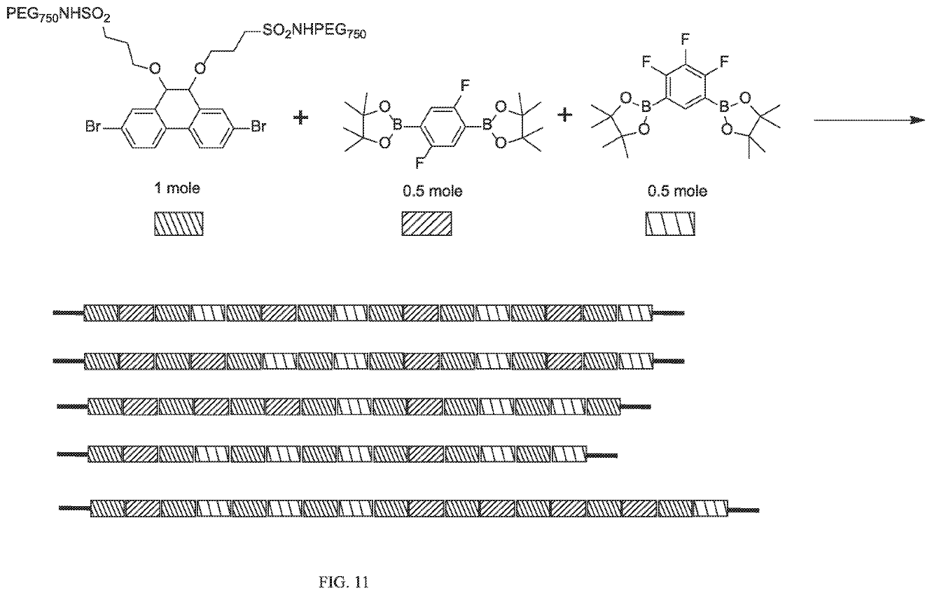

The modifying units M¹ and M² can be evenly or randomly arranged along the polymer chain. For example, FIG. 11 shows an exemplary scheme for a UV-absorbing polymer according to the disclosure wherein two different modifying units M¹ and M² are distributed randomly in alternate positions to the repeating DHP units. The number of segments is a cartoon representing average Mn of individual polymer molecules of different chain lengths.

Each M¹ and M² can each be independently selected from:

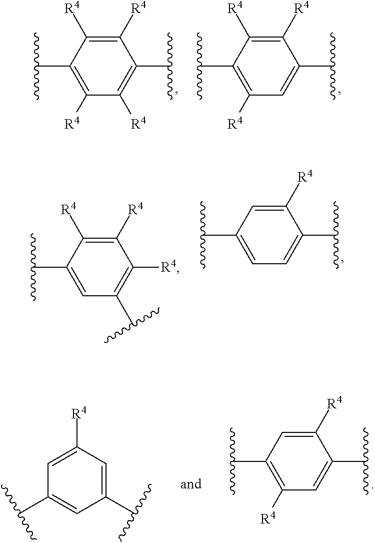

wherein M¹ and M² are different.

In some embodiments, the disclosure provides a UV-absorbing polymer according to Formula XIV:

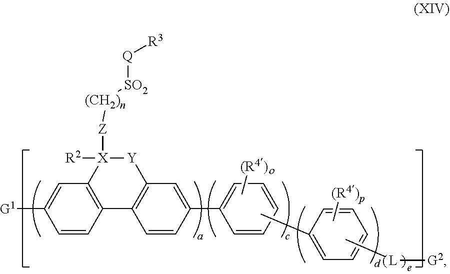

wherein each R², R³, G¹, G², L, Q, X, Y, Z, a, b, c, e, n, and m is independently as described herein; each R^{4′} is independently selected from R⁴ and at least one R^{4′} is not H; each R^{4″} is independently selected from R⁴ and at least one R^{4″} is not H; R⁹ is C₁-C₈ alkyl; each f is independently an integer from 0 to 50, or 10-20; each o is independently an integer selected from 1, 2, 3, or 4; and each p is independently an integer selected from 1, 2, 3, or 4. In some examples, the UV-absorbing polymer according to Formula XIV has a near ultraviolet excitation spectrum and/or absorbance maximum in a range of from 300 nm to 400 nm, or from 350 nm to 400 nm.

In the Formulas described herein, each linker L can be an aryl or heteroaryl group evenly or randomly distributed along the polymer main chain and that is substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof, which can be conjugated to another substrate, acceptor dye, molecule or binding partner. Each L can be independently selected from:

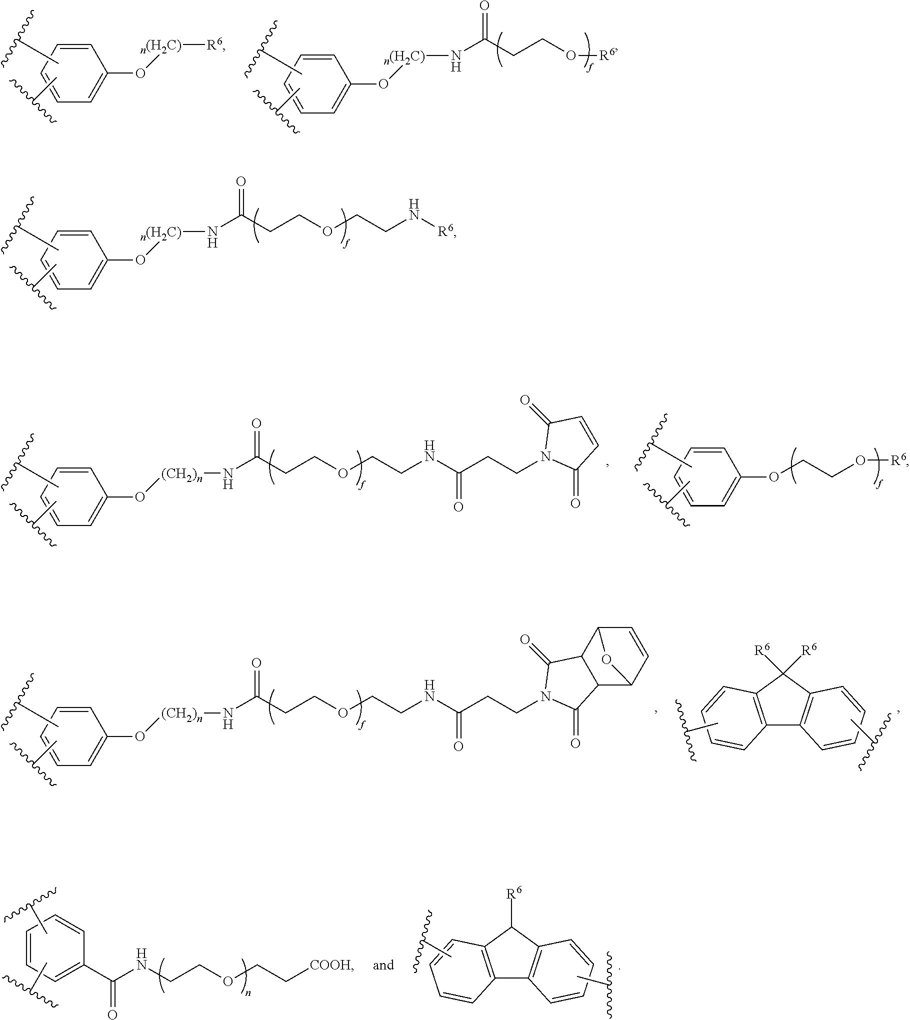

Each R⁶ can be independently selected from H, OH, SH, NHCOO-t-butyl, (CH₂)ₙCOOH, (CH₂)ₙCOOCH₃, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙCH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₁-C₁₂ alkene, C₂-C₁₂ alkyne, C₃-C₁₂ cycloalkyl, C₁-C₁₂ haloalkyl, C₁-C₁₂ (hetero)aryl, C₁-C₁₂ (hetero)arylamino, optionally substituted benzyl, halogen, hydroxyl, C₁-C₁₂ alkoxy, or (OCH₂CH₂)OCH₃. Each f can independently be an integer from 0 to 50, 10 to 20, or 11 to 18. Each n can independently bean integer from 1 to 20.

The UV-absorbing polymers can include capping units represented in the Formulas herein as G¹ and G². Capping units G¹ and G² can each independently be an unmodified polymer terminus and a modified polymer terminus For example, G¹ and G² can each be independently selected from hydrogen, halogen, alkyne, optionally substituted aryl, optionally substituted heteroaryl, halogen substituted aryl, silyl, diazonium salt, triflate, acetyloxy, azide, sulfonate, phosphate, boronic acid substituted aryl, boronic ester substituted aryl, boronic ester, boronic acid, optionally substituted dihydrophenanthrene (DHP), optionally substituted fluorene. Optionally substituted aryl, heteroaryl, fluorene, or DHP can be substituted with one or more pendant chains terminated with a functional group selected from amine, carbamate, carboxylic acid, carboxylate, maleimide, activated ester, N-hydroxylsuccinimidyl, hydrazine, hydrazide, hydrazone, azide, alkyne, aldehyde, thiol, and protected groups thereof for conjugation to a substrate or binding partner. In some instances, at least one capping unit G¹ or G² is conjugated to a substrate or binding partner. Capping units G¹ and G² can each be independently selected from optionally substituted dihydrophenanthrene (DHP), optionally substituted fluorene, aryl substituted with one or more pendant chains terminated with a functional group, and a heteroaryl substituted with one or more pendant chains terminated with a functional group. In some examples, capping units G¹ and G² can each be independently selected from:

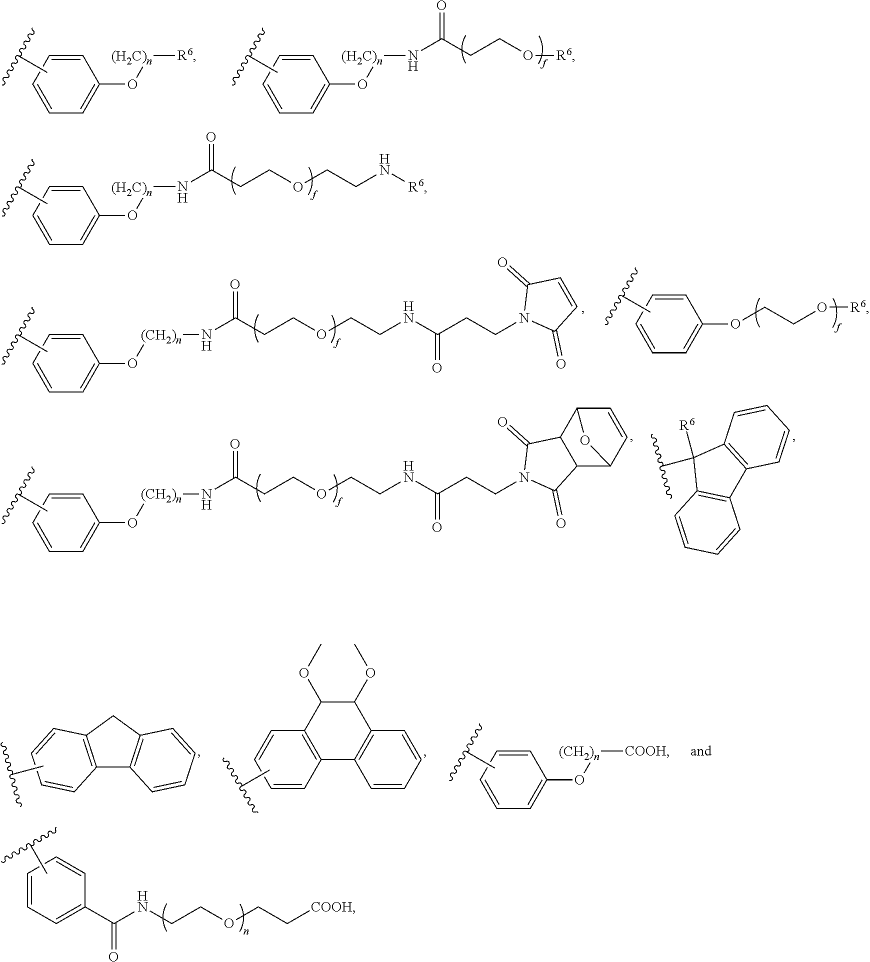

wherein each R⁶ can be independently selected from H, OH, SH, NHCOO-t-butyl, (CH₂)ₙCOOH, (CH₂)ₙCOOCH₃, (CH₂)ₙNH₂, (CH₂)ₙNH—(CH₂)ₙ—CH₃, (CH₂)ₙNHCOOH, (CH₂)ₙNHCO—CH₂)ₙ—CO—(CH₂)ₙ—CH₃, (C₁₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(C₁₂)ₙCOO(CH₂)ₙCH₃, (CH₂)ₙ(OCH₂CH₂)OCH₃, N-maleimide, halogen, C₂-C₁₂ alkene, C₂-C₁₂ alkyne, C₃-C₁₂ cycloalkyl, C₁-C₁₂ haloalkyl, C₁-C₁₂ (hetero)aryl, C₁-C₁₂ (hetero)arylamino, optionally substituted benzyl, halogen, hydroxyl, C₁-C₁₂ alkoxy, or (OCH₂CH₂)_{f}OCH₃. Each f can independently be an integer from 0 to 50, or 11 to 18. Each n can independently be an integer from 1 to 20 (e.g., 1, 2, 3, 4, 5, 6, 7, 8, 9, 10, 11, 12, 13, 14, 15, 16, 17, 18, 19, or 20).

The variables a, c, d, and e define the mol % of each unit within the structure which each can be evenly or randomly repeated and where a is a mol % from 10 to 100%, c is a mol % from >0 to 90%, each d is a mol % from 0 to 90%, and each e is a mol % from 0 to 25%. The variable a can be a mol % from 10 to 100%, 25% to 75%, 35% to 65%, 45% to 55%, or greater than or equal to 10%, 15, 20, 25, 30, 35, 40, 42, 44, 45, 46, 47, 48, 49, 50, 51, 52, 53, 54, 55, 56, 58, 60, 65, 70, 75, 80, 85, 90, or 95%. The variable c can be a mol % from >0 to 90%, 5% to 80%, 10% to 40%, 15% to 35%, 20% to 30%, or less than or equal to 90% but greater than or equal to 1%, 2, 3, 4, 5, 6, 8, 10, 12, 14, 16, 18, 20, 21, 22, 23, 24, 25, 26, 27, 28, 29, 30, 35, 40, 45, 50, 55, 60, 65, 70, 75, 80, or 85%. The variable d can be a mol % from 0 to 90%, 5% to 80%, 10% to 40%, 15% to 35%, 20% to 30%, or less than or equal to 90% but greater than or equal to 0%, 1, 2, 3, 4, 5, 6, 8, 10, 12, 14, 16, 18, 20, 21, 22, 23, 24, 25, 26, 27, 28, 29, 30, 35, 40, 45, 50, 55, 60, 65, 70, 75, 80, or 85%. The variable e can be a mol % from 0 to 25%, 0% to 20%, 0% to 10%, or less than or equal to 25% but greater than or equal to 0%, 1, 2, 3, 4, 5, 6, 7, 8, 9, 10, 12, 14, 16, 18, 20, 22, or 24%.

The polymer can have the structure of Formula IX:

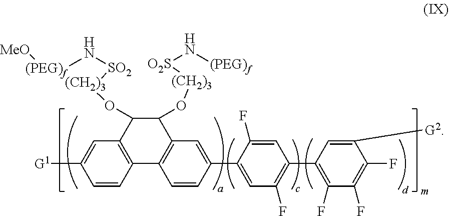

The variable f can independently be an integer from 0 to 50. The units in the polymer structure represented in Formula IX can occur in any suitable order within the polymer backbone, such as the same or different order as shown in Formula IX. For example, the units in the polymer structure represented in Formula IX can occur in the order shown in Formula X:

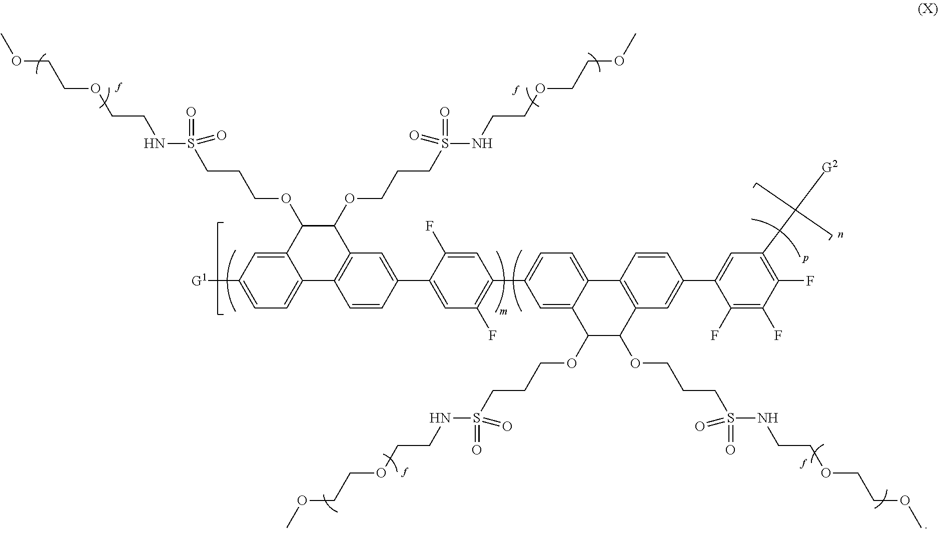

For example, the units in the polymer structure represented in Formula IX or X can occur in the order shown in Formula XI:

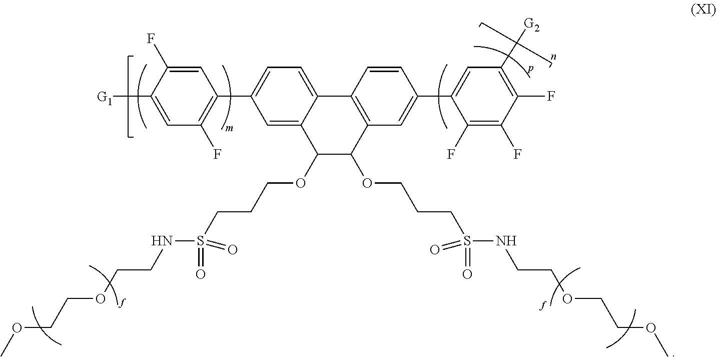

In Formula X and XI, the variables m, p, and n define the mol % of each unit within the structure. The variable m can be the same as described herein for Formulas I-IX or XIV. In the Formulas described herein, the groups M¹ and M² can have any suitable molar ratio to one another in the UV-absorbing polymer. For example, a molar ratio of M¹ to M² groups can be 0.5:1 to 1.5:1, 0.7:1 to 1.3:1, 0.9:1 to 1.1:1, about 1:1, or less than or equal to 1.5:1 but greater than or equal to 0.5:1, 0.6:1, 0.7:1, 0.8:1, 0.9:1, 1:1, 1.1:1, 1.2:1, 1.3:1, or 1.4:1.

The UV-absorbing polymer can have an absorption maximum within a range of from 300 nm to 400 nm, 320 nm to 380 nm, 330 n to 380 nm, 335 nm to 380 nm, 340 nm to 380 nm, 350 nm to 380 nm, 350 nm to 375 nm, 340 nm to 360 nm, 345 urn to 356 un, or less than or equal to 380 nm but greater than or equal to 320, 322, 324, 326, 328, 330, 332, 334, 336, 338, 340, 342, 344, 345, 346, 347, 348, 349, 350, 351, 352,353, 354, 355, 356, 358, 360, 362, 364, 366, 368, 370, 372, 374, 376, or 378 nm. The polymer can have an emission maximum of about 380 nm or higher, or within a range of about 380 nm to about 1000 nm, about 380 n to about 800 nm, 380 nm to 430 nm, 406 nm to 415 nm, or less than or equal to 430 nm but greater than or equal to 380 nm, 382, 384, 386, 388, 390, 392, 394, 396, 398, 400, 402, 404, 406, 407, 408, 409, 410, 411, 412, 413, 414, 415, 416, 418, 420, 422, 424, 426, or 428 nm. In some instances the emission maximum may be greater than 1000 nm.

The UV-absorbing polymer dye may have any appropriate molecular weight (MW) which may be expressed, for example, in g/mol or kilodaltons (kDa). In some cases, the MW of the UV-absorbing polymer dye may be expressed as an average molecular weight. In some instances, the UV polymer dye may have an average molecular weight in a range of from 1,000 to 500,000, such as from 2,000 to 400,000, from 5,000 to 300,000, from 10,000 to 200,000, from 25,000 to 175,000, from 30,000 to 150,000, from 40,000 to 150,000, or even an average molecular weight of from 50,000 to 100,000. The UV-absorbing polymer dye may have an average molecular weight of 20 to 150 kDa, 30 to 130 kDa, 40 to 120 kDa, 50 to 100 kDa, or 60-70 kDa.

Monomers for preparing UV-absorbing polymers of the present disclosure can include a dihydrophenanthrene (DHP)-based monomer, such as a 9,10-phenanthrenedione-based monomer and/or a fluorene-based monomer. For example, monomers of the present disclosure may include:

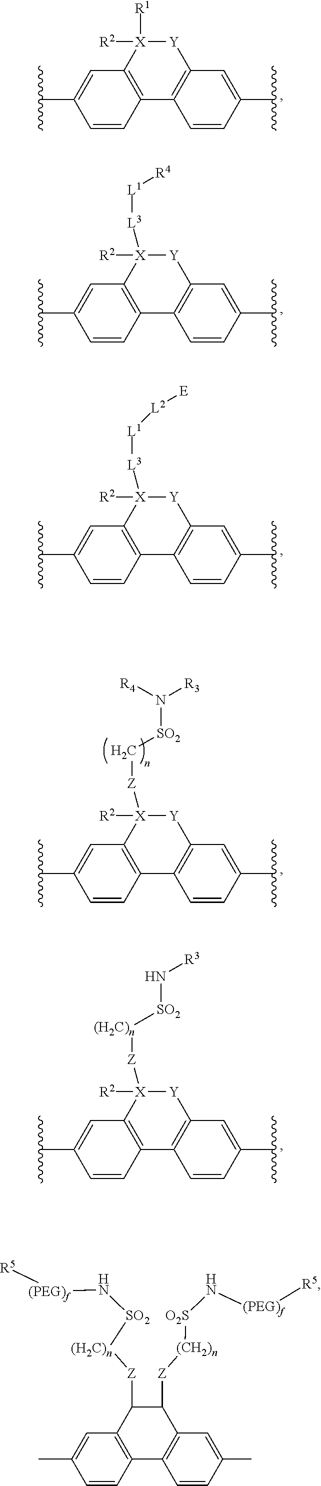

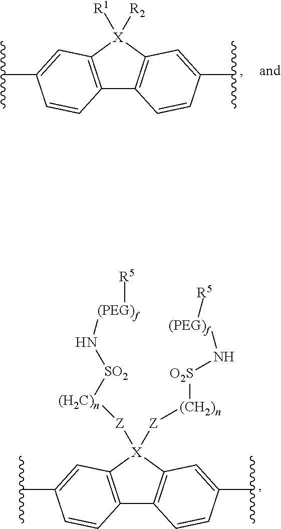

wherein both terminal ends of the monomers are independently or both a halogen atom, boronic ester or boronic acid, silyl, diazonium salt, triflate, acetyloxy, sulfonate, or phosphate which can undergo Pd or Nickel salt catalyst polymerization reactions. The variables R¹, R², X, Y, Z, n, R³, f, and R⁵ are as described herein.

In some embodiments, monomers of the present invention also include bridged monomers. For example, bridged monomers of the present disclosure can include:

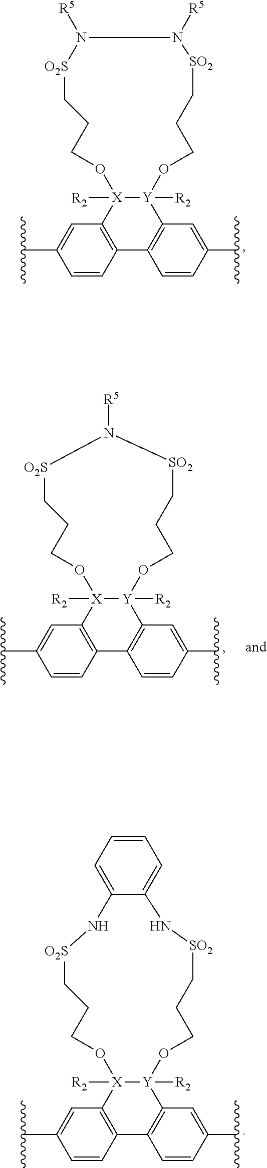

In various aspects of the present invention, the polymer further includes a binding partner linked to the polymer. In some aspects, the binding partner can be an antibody. A “binding partner” of the disclosure can be any molecule or complex of molecules capable of specifically binding to a target analyte. A binding partner of the disclosure includes, for example, proteins, small organic molecules, carbohydrates (including polysaccharides), oligonucleotides, polynucleotides, lipids, affinity ligand, antibody, antibody fragment, an aptamer, and the like. In some embodiments, the binding partner is an antibody or fragment thereof. Specific binding in the context of the present disclosure refers to a binding reaction which is determinative of the presence of a target analyte in the presence of a heterogeneous population. Thus, under designated assay conditions, the specified binding partners bind preferentially to a particular protein or isoform of the particular protein and do not bind in a significant amount to other proteins or other isoforms present in the sample.

When the binding partners are antibodies, they may be monoclonal or polyclonal antibodies. The term antibody as used herein refers to immunoglobulin molecules and immunologically active portions of immunoglobulin (Ig) molecules. Such antibodies can include polyclonal, monoclonal, mono-specific polyclonal antibodies, antibody mimics, chimeric, single chain, Fab, Fab′ and F(ab′)₂ fragments, Fv, and a Fab expression library.

In general, UV-absorbing polymers of the present disclosure can be conjugated to binding partners using techniques known to those of skill in the art or using methods known in the art in combination with methods described herein.

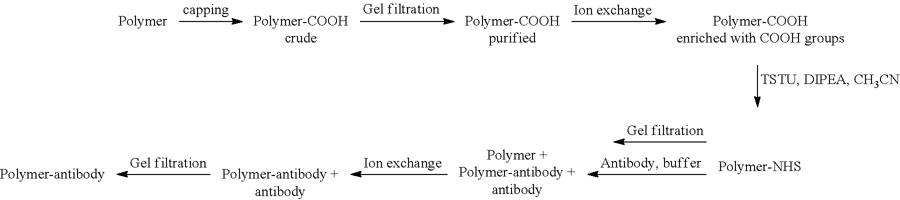

For example, preparation of polymer NHS ester can proceed as follows. Take 5 mg of the polymer in a clean vial and dissolve in 1 mL dry CH₃CN. To this add 15 mg TSTU and stir for 2 more minutes. To this add 100 μL DIPEA and continue stirring for overnight with the cap sealed with parafilm. Later, evaporate off the organic solvents in the reaction mixture. Dissolve the crude NHS in about 750 μL of 1×BBS buffer (pH 8.8) by a quick vortex and transfer it to the Zebra column 40K MWCO. Spin down the sample at 2200 RPM for 2 minutes and use the polymer NHS immediately.

Conjugation of polymer NHS with CD4 can proceed as follows. Take the polymer NHS in 1×BBS (˜800 μL) which was spun down, add to 0.6 mg of CD4 and mix with 100 μL of 0.5 M borate buffer (pH 9.0). Vortex quickly for 30 seconds and allow to mix for 3-4 hours in the coulter mix.

Purification of conjugate through Histrap HP column can proceed as follows. Approach 1: After the crude reaction purify the conjugate using a Histrap HP column. Load the sample using 1×PBS buffer and collect the unbound fraction. This can be done using 20CV of buffer. Later, change the buffer to wash the bound fraction which has both conjugate and free antibody. This can be done using 1×PBS with 0.25M imidazole running for 10CV. Approach 2: Histrap SP Sepharose F column. Equilibrate the column and load the sample using 20 mM citrate buffer pH 3.5 and collect the unbound fraction. This can be done using 20CV of buffer. Later, change the buffer to elute the bound fraction which has both conjugate and free antibody. This can be done using 20 mM Tris buffer pH 8.5 running for 20CV. Approach 3: Load the crude conjugate in a Tangential flow filtration system equipped with a 300K MWCO membrane. The conjugate can be washing using 1×PBS until the filtrate shows no absorption at 405 nm. Later, the compound is concentrated.

Purification of conjugate through a SEC column can proceed as follows. Load the crude conjugate containing free antibody to the Size Exclusion Column, using 1×PBS. Pool the tubes after checking the absorption spectra and concentrate in a Amicon Ultra-15 having a 30 KDa MWCO centrifugal concentrator.

### Method for Detecting an Analyte in a Sample

The present disclosure provides a method for detecting an analyte in a sample including: contacting a sample that is suspected of containing the analyte with a binding partner conjugated to a UV-absorbing polymer (including, but not limited to, a LV-absorbing polymer-tandem polymer) of the present disclosure (e.g., a UV-absorbing polymer as shown in Formulas I-XI, or XIV, for example any one of Formulae I, II, III, IV, V, VI, VII, VIII, IX, X, XI, and/or XIV, according to the present disclosure, and polymer-tandem dyes thereof). The binding partner is capable of interacting with the analyte. If the analyte is present, the binding partner and analyte can form a polymer dye conjugate complex. The binding partner can optionally be bound to a substrate. The binding partner can be a protein, peptide, affinity ligand, antibody, antibody fragment, sugar, lipid, nucleic acid, or an aptamer. A light source is applied to the sample that can excite the polymer and light emitted from the conjugated polymer complex is detected. In the typical assay, UV-absorbing polymers of the disclosure are excitable with a light having a wavelength of 320 nm to 380 nm, 340 nm to 360 nu, 345 nm to 356 nm, or less than or equal to 380 nm but greater than or equal to 320, 322, 324, 326, 328, 330, 332, 334, 336, 338, 340, 342, 344, 345, 346, 347, 348, 349, 350, 351, 352, 353, 354, 355, 356, 358, 360, 362, 364, 366, 368, 370, 372, 374, 376, or 378 nm. The emitted light is typically a wavelength of 380 nm to 430 nm, 406 nm to 415 nm, or less than or equal to 430 nm but greater than or equal to 380 nm, 382, 384, 386, 388, 390, 392, 394, 396, 398, 400, 402, 404, 406, 407, 408, 409, 410, 411, 412, 413, 414, 415, 416, 418, 420, 422, 424, 426, or 428 nm.

A method is provided for detecting an analyte in a sample comprising: adding at least one polymer dye conjugate to a composition according to the disclosure to form a polymer dye conjugate composition; contacting a biological sample that is suspected of containing an analyte with the polymer dye conjugate composition to form a fluorescent polymer dye conjugate complex with the analyte; applying a light source to the sample that can excite the at least one fluorescent polymer dye conjugate complex; and detecting light emitted from the fluorescent polymer dye conjugate complex.

in some embodiments, the light from the light source has a wavelength between about 340 nm and about 450 nm. In some embodiments, the emitted light has a wavelength between about 380 nm and about 1000 nm, or 380 and 800 nm. The detecting light may further comprise analyzing by flow cytometry to obtain a first flow cytometry plot, wherein the first flow cytometry plot exhibits one or more of the group consisting of: decreased non-specific interaction of polymer dye conjugates; and decreased aggregation of polymer dye conjugates, when compared to a second flow cytometry plot obtained comprising contacting the biological sample with a composition without the nonionic surfactant and without the UV-absorbing polymer dye or quenched UV polymer dye.

The biological sample in the methods of the present disclosure can be, for example, blood, bone marrow, spleen cells, lymph cells, bone marrow aspirates (or any cells obtained from bone marrow), urine (lavage), serum, saliva, cerebral spinal fluid, urine, amniotic fluid, interstitial fluid, feces, mucus, or tissue (e.g., tumor samples, disaggregated tissue, disaggregated solid tumor). In some embodiments, the sample is a blood sample. In some embodiments, the blood sample is whole blood. The whole blood can be obtained from the subject using standard clinical procedures. In some embodiments, the sample is a subset of one or more cells of whole blood (e.g., erythrocyte, leukocyte, lymphocyte, phagocyte, monocyte, macrophage, granulocyte, basophil, neutrophil, eosinophil, platelet, or any cell with one or more detectable markers). Examples of lymphocyte cells can include T cells, B cells, or NK cells. In some embodiments, the sample can be from a cell culture.

The subject can be a human (e.g., a patient suffering from a disease), a commercially significant mammal, including, for example, a monkey, cow, or horse. Samples can also be obtained from household pets, including, for example, a dog or cat. In some embodiments, the subject is a laboratory animal used as an animal model of disease or for drug screening, for example, a mouse, a rat, a rabbit, or guinea pig.

An “analyte” as sued herein refers to a substance, e.g., a molecule, whose abundance/concentration is determined by some analytical procedure. For example, in the present disclosure, an analyte can be protein, peptide, nucleic acid, lipid, carbohydrate, or small molecule.

The target analyte can be, for example, nucleic acids (DNA, RNA, mRNA, tRNA, or rRNA), peptides, polypeptides, proteins, lipids, ions, monosaccharides, oligosaccharides, polysaccharides, lipoproteins, glycoproteins, glycolipids, or fragments thereof. In some embodiments, the target analyte is a protein and can be, for example, a structural microfilament, microtubule, and intermediate filament proteins, organelle-specific markers, proteasomes, transmembrane proteins, surface receptors, nuclear pore proteins, protein/peptide translocases, protein folding chaperones, signaling scaffolds, ion channels, and the like. The protein can be an activatable protein or a protein differentially expressed or activated in diseased or aberrant cells, including but not limited to transcription factors, DNA and/or RNA-binding and modifying proteins, nuclear import and export receptors, regulators of apoptosis or survival, and the like. The analyte can be a protein expressed on cell surface.

Assay systems utilizing a binding partner and a fluorescent label to quantify bound molecules are well known. Examples of such systems include flow cytometers, scanning cytometers, imaging cytometers, fluorescence microscopes, and confocal fluorescent microscopes.

In some embodiments, the method can be configured to flow cytometry. Flow cytometry can be used to detect fluorescence. A number of devices suitable for this use are available and known to those skilled in the art. Examples include BCI Navios, Gallios, Aquios, and CytoFLEX flow cytometers.

In other embodiments, the method can be configured as an immunoassay. Examples of immunoassays useful in the present disclosure include a fluoroluminescence assay (FLA) and the like. The assays can also be carried out on protein arrays.

When the binding partners are antibodies, antibody or multiple antibody sandwich assays can also be used. A sandwich assay refers to the use of successive recognition events to build up layers of various binding partners and reporting elements to signal the presence of a particular analyte. Examples of sandwich assays are disclosed in U.S. Pat. No. 4,486,530 and in the references noted therein.

In some embodiments, the method can include providing additional binding partners (e.g., more than one binding partner) for detecting additional analytes simultaneously.

### Staining Buffer Compositions

Polymer dyes (PDs) are hydrophobic and have large apparent molecular weights which make them prone to aggregation in aqueous buffer. Consequently, when polymer dyes are conjugated to antibodies, the resulting conjugates also have high propensity to interact with each other and/or with other polymer dye conjugates present in the same sample. When more than one polymer dye conjugates are used for staining the same sample, non-specific interaction between the polymer dyes normally occurs which can result in under-compensation of data and may cause incorrect data analysis. Prior art competitive staining buffer compositions are available commercially; however, these staining buffers are somewhat dye-specific, and exhibit diminished effectiveness in preventing dye-to-dye interactions across different dye classes in a multi-color panel. A universal staining buffer capable of suppressing dye-to-dye interactions from all types of polymer dye conjugates is desirable.

In order to develop a universal staining buffer suitable for use with a multi-color panel of polymer dye conjugates, various detergents, PEGs, amino acids, DNA, peptides, proteins, polymers (violet polymers, ultraviolet polymers, etc.), urea, and the like were tested alone or in combination.

The disclosure provides staining buffer compositions capable of reducing, substantially reducing or eliminating polymer-polymer interactions in a multi-color panel across dye classes. A universal staining buffer solution has been developed that is suitable for use in multi-color panels including different polymer dye conjugates, e.g., from different commercial vendors. The multi-color panel may include one or more, or two or more different types of polymer dye conjugates.

The polymer dye conjugates in the multi-color panels may, in some embodiments, be fluorescent dye conjugates that can be excited by, for example, ultraviolet (e.g., 351 nm, 355 nm, 375 nm, 334-364 nm, 351-356 nm), violet (e.g., 405 nm, 407 ma, 414 ma, 395-425 nm), blue (e.g., 436 nm, 458 nm), blue-green (e.g., 488 nm), green (e.g., 514 nm, 532 nm, 541 nm, 552 nm), yellow-green (e.g., 561 nm, 563 nm), yellow (e.g., 568 nm), red (e.g., 627-640 nm, 633 nm, 637 nm, 640 nm, 647 nm), and/or near infrared lasers (e.g., 673 nm, 750 nm, 780 nm, or in a range of from 660-800 nm).

The disclosure provides a staining buffer composition comprising a UV polymer dye or quenched UV polymer dye and a nonionic surfactant for reducing or preventing non-specific interactions between polymer dye conjugates. A composition is provided for use with at least one fluorescent polymer dye conjugated to a binding partner for use in staining a biological sample, the composition comprising: at least one UV-absorbing polymer dye or quenched UV-absorbing dye; a nonionic surfactant; and optionally a biological buffer. In some embodiments, the UV polymer dye or quenched UV polymer dye may be a dye according to the disclosure. The composition reduces non-specific binding of the at least one fluorescent polymer dye conjugate, when compared to the at least one fluorescent polymer dye conjugate in the absence of the composition.

The staining buffer composition may be added to a multi-color panel of polymer dye conjugates before staining cells and can effectively reduce or prevent non-specific interactions of the polymer dye conjugates, for example, in a flow cytometric analysis (FCA) of a biological sample. The staining buffer composition has been found to substantially decrease non-specific polymer dye conjugate interactions in a multi-color dye conjugate panel. This was evidenced in a FCA of a processed whole blood sample when compared to the same sample without the LV-absorbing polymer dye or quenched UV polymer dye and without the nonionic surfactant.

The ready-to-use staining buffer compositions of the disclosure are universal solutions that work for all types of polymer dye conjugates across all polymer dye classes, including, for example, violet-excitable polymer dye conjugates, ultra-violet-excitable polymer dye conjugates, blue-excitable polymer dye conjugates, red-excitable polymer dye conjugates etc. The staining buffer compositions have been found to substantially reduce or completely eliminate the non-specific interactions that may occur during cell staining with multiple polymer dye conjugates.

### UV-Absorbing Polymer Dyes

The staining buffer composition according to the disclosure may comprise one or more UV-absorbing polymer dyes, one or more UV-absorbing tandem polymer dyes, and/or one or more quenched LV-absorbing polymer dyes. The UV-absorbing polymer dye, UV-absorbing tandem polymer dye, or quenched UV-absorbing polymer dye for use in the staining buffer composition may, in some embodiments, be a DHP-based dye, a fluorene-based dye, a binapthyl-based dye, a carbazole-based dye, an oxepine-based dye (e.g., a fluorenooxepine-based dye), or combinations thereof. The UV polymer dye, UV tandem polymer dye, or quenched UV polymer dye for use in the staining buffer composition may have one or more water-solubilizing moieties. The UV-absorbing polymer dye, UV-absorbing tandem polymer dye, or quenched UV-absorbing polymer dye may be conjugated to a binding partner. The UV-absorbing polymer dye, LV-absorbing tandem polymer dye, or quenched UV-absorbing polymer dye may be a water-soluble UV-absorbing polymer dye. The UV-absorbing polymer dye, LV-absorbing tandem polymer dye, or quenched LV-absorbing polymer dye may be according to the present disclosure.

Ultraviolet (UV) is a region of the electromagnetic spectrum from about 10 nm to about 400 nm. In some examples, the present disclosure provides UV-absorbing polymer dyes having a near ultraviolet excitation spectrum and/or a near UV absorption maximum. In some examples, the present disclosure provides water-soluble UV-absorbing polymer dyes having a near ultraviolet excitation spectrum and/or a near LV absorption maximum. Near ultraviolet (UV) is a region of the electromagnetic spectrum from about 300 nm to about 400 nm, such as from 350 n to 400 un. The term “near ultraviolet excitation spectrum” can refer to the absorption spectrum of a UV-absorbing polymer dye that has a full width at half maximum (FWHM) defining a wavelength range that is inside the near UV region of the electromagnetic spectrum.

The UV polymer dye for use in the staining buffer composition may have a structure according to any of Formulae I, II, III, IV, V, VI, VII, VIII, IX, X, XI, and/or XIV. In some instances, the UV polymer dye may be a UV-absorbing polymer tandem dye (“UV polymer tandem dye”), e.g., a quenched UV-absorbing polymer dye (“quenched UV polymer dye”), according to any of Formulae I, II, III, IV, V, VI, VII, VIII, IX, X, XI, and/or XIV. In some embodiments, the UV-absorbing polymer dye does not comprise a binding partner. In some embodiments, the quenched UV polymer dye does not comprise a binding partner.

In some embodiments, the UV polymer dye or quenched UV polymer dye for use in the staining buffer may comprise a UV polymer dye known in the art, such as, for example, as taught in U.S. Pat. Nos. 9,719,998; 10,228,375; 11,119,107; 10,605,813; US 2019/0194467A1 or WO2022/13198, each of which are incorporated herein by reference in their entireties.

In some embodiments, the staining buffer according to the disclosure includes a UV polymer dye or quenched UV polymer dye comprising a binding partner, In some embodiments, the UV polymer dye or quenched UV polymer dye for use in the staining buffer does not comprise a binding partner.

Quenched polymers may comprise a polymer dye according to the disclosure comprising one or more, or a multiplicity of quenching moieties, for example, 1-50, 2-25, or 5-8 quenching moieties. In some embodiments, the quenched polymer exhibits a quantum yield (QY) no more than 0.1, or no more than 0.06, or no more than 0.056, no more than 0.05, no more than 0.02, or no more than 0.015Φ.

In some embodiments, the UV-absorbing dye may have an average molecular weight in the range of from about 5 to about 150 kDa, about 10 to about 150 kDa, about 20 to about 150 kDa, about 40 to about 120 kDa, about 50 to about 100 kDa, or about 60 to about 70 kDa.

The staining buffer composition may include 0.01-10 mg/nL, 0.02-5 mg/mL, 0.05-2 mg/mL, 0.1-1 mg/nL, 0.2-0.8 mg/mL, or about 0.5 mg/mL of a UV polymer dye or quenched UV polymer according to the disclosure. The amount of LV polymer dye or quenched UV polymer per test may be from about 1 to about 50 ug/test, about 2 to about 30 ug/test, or about 5 to about 20 ug/test.

### Nonionic Surfactants

The composition may comprise one or more nonionic surfactants. A sufficient amount of the nonionic surfactant can be included to prevent aggregation of polymer dye conjugates. Non-limiting examples of nonionic surfactants includes polysorbate nonionic surfactants such as, TWEEN® 20 and TWEEN® 80 an ether-linked nonionic surfactants such as, for example, polyoxyethylene glycol alkyl ether (BRIJ), a polyoxyethylene glycol octylphenol ether (TRITON), or a polyoxyethylene nonylphenyl ether (IGEPAL) surfactant. In some embodiments, the surfactant is a poloxamer nonionic surfactant.

The term “poloxamer nonionic surfactant” or “poloxamer” refers to a polyethylene oxide-polypropylene oxide-polyethylene oxide (PEG-PPG-PEG) nonionic triblock copolymer. Poloxamer nonionic surfactant are known by trade names, for example, PLURONIC® (BASF) nonionic surfactants, Kolliphor® (BASF) nonionic surfactants, and Synperonic™ (CRODA) nonionic surfactants. Non-limiting examples of PLURONIC® surfactants may include, for example, PLURONIC® F68, F77, F87, F98, F108, F123, F127, P103, P104, P105, P123, PE 3100, PE4300, PE6100, PE6200, PE6400, PE6800, PE8100, PE9400, PE10100, PE10400, PE10500 (BASF Corporation). Poloxamer surfactants include nonionic triblock copolymers such as PEO) characterized by a central hydrophobic chain of polyoxypropylene (poly(propylene oxide)) flanked by two hydrophilic chains of polyoxyethylene (poly(ethylene oxide)).

The staining buffer composition may include a nonionic surfactant that is poly(ethylene oxide)-poly(propylene oxide)-poly(ethylene oxide) triblock copolymer. Exemplary nonionic triblock copolymers may comprise a structure according to Formula XII,

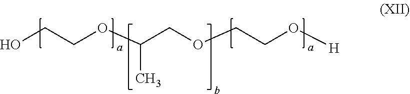

wherein each a is independently an integer independently in the range of 2-130, and b is an integer in the range of 15-67, In some embodiments, a is in the range of 50-100 and b is in the range of 20-40. In some embodiments, a is in the range of 70-90 and b is in the range of 25-30. The nonionic surfactant may be poloxamer 188.

Non-limiting examples of poloxamers may include poloxamer 188, also known as Pluronic p-68, or KOLLIPHOR® P188, e.g., having a=80 and b=27. Other poloxamers include poloxamer 338, also known as Synperonic™ PE/F108, poloxamer 407, also known as Synperonic™ PE/F127, poloxamer 331, also known as Synperonic™ PE/L101.

Because the lengths of the polymer blocks can be customized, many different poloxamers exist that have slightly different properties. Poloxamer copolymers are commonly named with the letter “P” (for poloxamer) followed by three digits, the first two digits X 100 give the approximate molecular mass of the polyoxypropylene core, and the last digit X 10 gives the percentage polyoxyethylene content (e.g., P407=Poloxamer with a polyoxypropylene molecular mass of 4,000 g/mol and a 70% polyoxyethylene content). For the Pluronic and Synperonic poloxamer tradenames, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobic chain; and the last digit X 10 gives the percentage polyoxyethylene content (e.g., F-68 indicates a polyoxypropylene molecular mass of 1,800 g/mol and a 80% polyoxyethylene content).

The term “PLURONIC® F68,” “Pluronic F-68”, or “PF-68”, also known as poloxamer 188, refers to poly(ethylene glycol)-block-poly(propylene glycol)-block poly(ethylene glycol) copolymer with an average molecular weight, avg. Mn, of 8350-8400.

The term “PLURONIC® F127” also known as poloxamer 407 refers to a triblock copolymer consisting of a central hydrophobic block of polypropylene glycol flanked by two hydrophilic blocks of polyethylene glycol (PEG). The approximate lengths of the two PEG blocks is 101 repeat units, while the approximate length of the propylene glycol block is 56 repeat units. This is also known by the Croda trade name Synperonic PE/F 127, of avg. 12,600 g/mol. The term “PLURONIC® P108” refers to poly(ethylene glycol)-block-poly(propylene glycol)-block poly(ethylene glycol), avg. Mn ˜14,600, The term “PLURONIC® P103” refers to poly(ethylene glycol)-block-poly(propylene glycol)-block poly(ethylene glycol), of avg. Mw ˜4,950. The term “PLURONIC® P104” refers to poly(ethylene glycol)-block-poly(propylene glycol)-block poly(ethylene glycol), of avg. Mw ˜5,900. The term “PLURONIC® P123” refers to poly(ethylene glycol)-block-poly(propylene glycol)-block poly(ethylene glycol), of avg. Mn about ˜5,800.

An exemplary poloxamer surfactant includes, but is not limited to, Pluronic F-68. PF-68 is a nonionic triblock copolymer polyoxyethylene oxide-polyoxypropylene oxide-polyoxyethylene oxide (PEO-PPO-PEO) such as poloxamer 188. The concentration of the surfactant used can be determined empirically (i.e., titrated such that precipitation of the conjugates does not occur). In some embodiments, the staining buffer composition may include a nonionic surfactant such as a poloxamer surfactant. The nonionic surfactant may be Pluronic F-68. In some instances, the nonionic surfactant may be used alone (i.e., without a UV dye or UV quenched dye in the buffer) to reduce or prevent nonspecific interactions. The nonionic surfactant may be present in the composition, for example, a working concentration staining buffer composition (IX), within a range of 0.1%-20%, 0.1-15%, 0.2-9%, 0.5-8%, or 1-7% (wt/vol). The nonionic surfactant may be present at final a concentration (wt/vol) of about 0.1-2%, 0.5-1.5%, or about 0.1%, 0.2%, 0.3%, 0.4%, 0.5%, 0.6%, 0.7%, 0.8%, 0.9% or about 1%, 2%, 3%, 4%, 5%, 6%, 7%, 8%, or 9%, 10%, 11%, 12%, 13%, 14%, 15%, 16%, 17%, 18%, 19% or any value in between per test. The nonionic surfactant may be present in a concentrated staining composition (10×), for example, within a range of 1-90%, 1-80%, 1-70%, 2-60%, 3-50%, 4-40%, or 5-25% (wt/vol). The nonionic surfactant may be present in a concentrated staining composition (10×), for example, within a range of 0.1-70%, 0.2-60%, 0.3-50%, 0.4-40%, or 0.5-25% (wt/vol).

### Biological Buffers

The term “biological buffer” refers to y compatible aqueous solution comprising one or more biological buffering agents which in a cell-free system maintains pH in the biological range of pH 6-8, 6.5-8, or 7-8. The aqueous solution may include ater for injection, milliQ water, or another form of highly purified water. The aqueous solution may include saline or alcohol. The biological buffer may include water and one or more biological buffering agents. The in certain embodiments, the biological buffering agents may include one or more of N-(2-acetamido)-aminoethanesulfonic acid (ACES), acetate, N-(2-acetamido)-iminodiacetic acid (ADA), 2-aminoethanesulfonic acid (AES), ammonia, 2-amino-2-methyl-1-propanol (AMP), 2-amino-2-methyl-1,3-propanediol (AMPD), N-(1,1-dimethyl-2-hydroxyethyl)-3-amino-2-hydroxypropanesulfonic acid (AMPSO), N,N-bis-(2-hydroxyethyl)-2-aminoethanesulfonic acid (BES), bicarbonate, N,N′-bis-(2-hydroxyethyl)-glycine, [Bis-(2-hydroxyethyl)-imino]-tris-(hydroxymethylmethane) (BIS-Tris), 1,3-Bis[tris(hydroxymethyl)-methylamino]propane (BIS-Tris-propane), boric acid, dimethylarsinic acid, 3-(Cyclohexylamino)-propanesulfonic acid (CAPS), 3-(cyclohexylamino)-2-hydroxy-1-propanesulfonic acid (CAPSO), carbonate, cyclohexylaminoethanesulfonic acid (CHES), citrate, 3-[N-Bis(hydroxyethyl)amino]-2-hydroxypropanesulfonic acid (DIPSO), formate, glycine, glycylglycine, N-(2-Hydroxyethyl)-piperazine-N′-ethanesulfonic acid (HEPES), lactate, N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, malate, maleate, 2-(N-Morpholino)-ethanesulfonic acid (MES), 3-N-Morpholino)-propanesulfonic acid (MOPS), 3-(N-Morpholino)-2-hydroxypropanesulfonic acid (MOPSO), phosphate, Piperazine-N,N′-bis(2-ethanesulfonic acid) (PIPES), Piperazine-N,N′-bis(2-hydroxypropanesulfonic acid) (POPSO), pyridine, polyvinylpyrrolidone (PVP), succinate, 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), 2-Aminoethanesulfonic acid, AES (Taurine), trehalose, triethanolamine (TEA), 2-[Tris(hydroxymethyl)-methylamino]-ethanesulfonic acid (TES), N-[Tris(hydroxymethyl)-methyl]-glycine (tricine), Tris(hydroxymethyl)-aminomethane (Tris), glyceraldehydes, mannose, glucosamine, mannoheptulose, sorbose-6-phosphate, trehalose-6-phosphate, iodoacetates, sodium citrate, sodium acetate, sodium phosphate, sodium tartrate, sodium succinate, sodium maleate, magnesium acetate, magnesium citrate, potassium phosphate, magnesium phosphate, ammonium acetate, ammonium citrate, ammonium phosphate, among other buffers. Representative buffering agents carbonic acid, tartaric acid, succinic acid, acetic acid, or phthalic acid; Tris tromethamine hydrochloride, or phosphate. Specific examples of conventional biological buffers may include phosphate-buffered saline (PBS), N-2-Hydroxyethylpiperazine-N′-2-hydroxypropanesulfonic acid (HEPES), 2-(N-morpholino)ethanesulfonic acid (MES), 3-(N-Morpholino) propanesulfonic acid (MOPS), 2-([2-Hydroxy-1,1-bis(hydroxymethyl)ethyl]amino)ethanesulfonic acid (TES), 3-[N-tris(Hydroxy-methyl) ethylamino]-2-hydroxyethyl]-1-piperazinepropanesulfonic acid (EPPS), Tris[hydroxymethyl]-aminomethane (THAM), 1,4-piperazinediethanesulfonic acid (PIPES), and Tris[hydroxymethyl]methyl aminomethane (TRIS) buffers. Conventional biological buffers may have a pK in the physiological range and function most efficiently in this range. The biological buffer may be in aqueous solution at a concentration of, for example, 10-100 mM, or 5-25 mM.

The biological buffer may be PBS. The term “PBS” refers to phosphate buffered saline which is an aqueous buffer which may contain sodium chloride, disodium hydrogen phosphate, potassium chloride, and/or potassium dihydrogen phosphate. For example, PBS may contain milliQ water or deionized water and 137 mM NaCl, 2.7 mM KCl, 10 mM Na₂HPO₄, 1.8 mM KH₂PO₄. The pH may be about pH 7.0-7.4. The PBS may or may not be preserved with an azide such as sodium azide. The PBS may be an isotonic solution. The buffer may be a PBA buffer. The PBA buffer may comprise PBS, BSA, and sodium azide. The PBA buffer may comprise 1×PBS, about 2 mg/mL BSA, and about 0.1% (wt/vol) sodium azide.

### Additional Components

The compositions of the disclosure can be used as staining buffer compositions, for example, in flow cytometry sample analysis and, as such, can comprise additional components, including, but not limited to, one or more of any suitable carriers, stabilizers, salts, chelating agents (e.g., EDTA), colorants, or preservatives. The compositions can also comprise an additional one or more surfactants (e.g., ionic surfactants, and zwitterionic surfactants). The ionic surfactant may be an anionic surfactant. The staining buffer composition can include formulation agents, such as suspending agents, solubalizing agents, s gents and/or dispersing agents. For example, the staining buffer composition may contain a carrier such as water, or a solvent such as, e.g., DMSO, DMF and acetronitrile as a solubilizing agent. The compositions can also include a pH adjusting agent, and typically the buffer is a salt prepared from an organic acid or base.

The staining buffer composition may include a protein stabilizer. The term “protein stabilizer” refers to a protein that serves to reduce non-specific binding, for example, to reduce cell-cell interactions, or to help prevent non-specific binding between an antibody and a non-target molecule. The compositions according to the disclosure may include a protein stabilizer. The protein stabilizer may be selected from one or more of the group consisting of a serum albumin, for example, a bovine serum albumin (BSA), a casein, or a gelatin. The protein stabilizer may be a BSA. The protein stabilizer may be present in the composition of the disclosure at a concentration of from 0.1-10 mg/mL, 0.5-5 mg/mL, 1-3 mg/mL or about 2 mg/mL.

The staining buffer composition may include any appropriate preservative. The preservative may be an antioxidant, biocide, or antimicrobial agent.

The preservative may be an inorganic salt. For example, the preservative may be sodium azide, 2-chloroacetamide, 2-methylisothiazolinone, salicylic acid, ProClin™, Kathon™ CG, 5-chloro-2-methyl-4-isothiazolin-3-one, or 2-methyl-4-isothiazolin-3-one. The preservative may be present in the composition of the disclosure at 0.01-0.5%, 0.05-0.3%, or about 0.1% (wt/vol).

The staining buffer compositions of the disclosure may include additional surfactants. Suitable additional surfactants that can optionally be used according to the methods described herein may include zwitterionic surfactants, such as betaines such alkyl betaines, alkylamidobetaines, amidazoliniumbetaines, sulfobetaines (INCI Sultaines), as well as a phosphobetaines. Examples of suitable zwitterionic surfactants include surfactants of the general formula R^{1′}[CO—X(CH₂)ⱼ]_{g}—N⁺(R^{2′})(R^{3′})—(CH₂)_{f}—[CH(OH)CH₂]ₕ—Y′, wherein R^{1′} is a saturated or unsaturated C₆₋₂₂ alkyl, such as a C₈₋₁₈ alkyl, a saturated C₁₀₋₁₆ alkyl or a saturated C₁₂₋₁₄ alkyl; X is NH, NR^{4′}, wherein R^{4′} is C₁₋₄ alkyl, O or S; j is an integer from 1 to 10, such as from 2 to 5 and 3; g is 0 or 1, R^{2′} and R^{3′} are each, independently, a C₁₋₄ alkyl, optionally hydroxy substituted by a hydroxyethyl group or a methyl; f is an integer from 1 to 4, such as 1, 2 or 3; h is 0 or 1; and Y is COO, SO₃, OPO(OR^{5′})(or P(O)(OR^{5′})O, wherein R^{5′} is H or C₁₋₄ alkyl.

Examples of suitable zwitterionic surfactants include alkyl betaines, such as those of the formula:

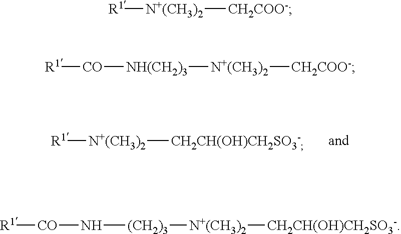

Examples of suitable betaines and sulfobetaines are the following (designated in accordance with INCI): almondamidopropyl betaine, apricotamidopropyl betaine, avocadamidopropyl betaine, babassuamidopropyl betaine, behenamidopropyl betaine, behenyl betaine, canolamidopropyl betaine, capryl/capramidopropyl betaine, carnitine, cetyl betaine, cocamidoetbyl betaine, cocamidopropyl betaine, cocamidopropyl hydroxysultaine, coco betaine, coco hydroxysultaine, coco/oleamidopropyl betaine, coco sultaine, decyl betaine, dihydroxyethyl oleyl glycinate, dihydroxyethyl soy glycinate, dihydroxyethyl stearyl glycinate, dihydroxyethyl tallow glycinate, dimethicone propyl of PG-betaine, drucamidopropyl hydroxysultaine, hydrogenated tallow betaine, isostearamidopropyl betaine, lauramidopropyl betaine, lauryl betaine, lauryl hydroxysultaine, lauryl sultaine, milk amidopropyl betaine, milkamidopropyl betaine, myristamidopropyl betaine, myristyl betaine, oleamidopropyl betaine, oleamidopropyl hydroxysultaine, oleyl betaine, olivamidopropyl betaine, palmamidopropyl betaine, palmitamidopropyl betaine, palmitoyl carnitine, palm kernel amidopropyl betaine, polytetrafluoroethylene acetoxypropyl betaine, ricinoleamidopropyl betaine, sesamidopropyl betaine, soyamidopropyl betaine, stearamidopropyl betaine, stearyl betaine, tallowamidopropyl betaine, tallowamidopropyl hydroxysultaine, tallow betaine, tallow dihydroxyethyl betaine, undecylenamidopropyl betaine and wheat germ amidopropyl betaine.

For example, coconut dimethyl betaine is commercially available from Seppic under the trade name of AMONYL 265®; and lauryl betaine is commercially available from Sigma-Aldrich under the trade name EMPIGEN BB®. A further example betaine is lauryl-imino-dipropionate commercially available from Rhodia under the trade name MIRATAINE H2C-HA®. Presence of optional zwitterionic surfactant in staining buffer composition may decrease non-specific binding in a biological sample, for example, may decrease non-specific binding to monocytes or other blood components. The optional zwitterionic surfactant may be present in the composition at 0-0.5%, 0.01-0.3%.

### Compositions

Staining buffer compositions are provided for decreasing polymer-polymer interactions between polymer dye conjugates and decreasing dye conjugate precipitation in a biological sample. Staining buffer compositions are provided for decreasing polymer-polymer interactions between polymer dye conjugates in a multi-color panel comprising two or more polymer dye conjugates.

The staining buffer composition may be used with one or more or a plurality of fluorescent polymer dye conjugates. The staining buffer composition of the disclosure may substantially reduce the non-specific binding between the plurality of fluorescent polymer dye conjugates.

The compositions according to the disclosure may be used with a mixture of dye conjugates comprising one or more, two or more, or three or more polymer dye conjugates prior to, concurrently with, or after adding to a biological sample for decreasing, substantially decreasing and/or preventing non-specific binding between polymer dye conjugates such as polymer-polymer interactions. The mixture of dye conjugates may include one or more, two or more, or three or more polymer dye conjugates.

A staining buffer composition is provided comprising a UV polymer dye or quenched UV polymer dye and a nonionic surfactant. The UV polymer dye or quenched UV polymer dye may be according to the disclosure. The staining buffer composition may comprise a UV polymer dye or quenched UV polymer dye according to the disclosure and a nonionic surfactant in a biological buffer

The staining buffer composition may provide 5-20 ug/test UV polymer dye and 0.1-2% (wt/vol)/test nonionic surfactant in a biological buffer.

The UV polymer dye may be any appropriate UV polymer dye or quenched UV polymer. The UV polymer dye may be according to the present disclosure. In some embodiments, the UV polymer dye does not include a binding partner. In some embodiments, the UV polymer dye does include a binding partner. In some embodiments, the binding partner is not an antibody or fragment thereof. The UV polymer may be a tandem UV polymer comprising one or more acceptor dyes. The UV polymer may be a quenched UV polymer comprising one or more quenching moieties. The quenched UV polymer dye may or may not include a binding partner. The nonionic surfactant may be a poloxamer nonionic surfactant. The poloxamer may be Pluronic F-68. Optionally the composition further comprises a protein stabilizer. Optionally the composition comprises a preservative. Optionally the composition comprises a zwitterionic surfactant. The biological buffer may be a PBS buffer. The protein stabilizer may be BSA. The preservative may be NaN₃. The composition is capable of reducing, substantially reducing, or eliminating polymer-polymer interactions in a multi-color panel.

Representative staining buffer compositions are provided in Table 1A.

TABLE 1A
Representative Staining Buffer Composition

| | Working Buffer (1X) | Concentrated (10X) |
|---|---|---|
| Component | Amount | Amount |
| UV polymer or | 0.3-0.7 mg/mL | 3-7 mg/mL |
| quenched UV | | |
| polymer | | |
| Nonionic Surfactant | 5-9% (wt/vol) | 50-90% (wt/vol) |
| Biological Buffer | 1X | 1X |
| Protein Stabilizer | 1-3 mg/mL | 10-30 mg/mL |
| Preservative | 0.01-0.03% (wt/vol) | 0.1-0.3% |

The staining buffer compositions of the disclosure are capable of reducing, substantially reducing, or eliminating polymer-polymer interactions in a multi-color panel.

The term “multi-color panel” refers to a mixture of dye conjugates that may include one or more, two or more, or three or more fluorescent polymer dye conjugates and optionally one or more, two or more, three or more fluorescent dye conjugates such as, for example, fluorescein, coumarin, cyanine, rhodamine dye conjugates, and the like, for example, FITC, PE, ECD, PC5, PC5.5, PC7, APC, AA700, AA750, PBE, Alexa Fluor® 488(AF488), AF532, AF647, AF700, AF750, Atlantis Bioscience CF®350 dye, CF®405S, CF®405, CF®405L, CF®430, CF®440, CF®450, CF®488A, CF®514, AAT Bioquest iFluor™ 488, iFluor™ 350, iFluor™ 405, mFluor™ Blue 570, mFluor™ Blue 580, mFluor™ Blue 590, mFluor™ Blue 620, mFluor™ Blue 630, mFluor™ Blue 660, ThermoFisher Scientific NovaFluor Blue 510, NovaFluor Blue 530, NovaFluor Blue 555, NovaFluor Blue 585, NovaFluor Blue 610/308, NovaFluor Blue 660/40S, NovaFluor Blue 660/1208, BioLegend® Kiravia Blue 520™, KrO dye conjugates and the like.

The term “fluorescent dye” refers to a dye comprising a light excitable fluorophore that can re-emit light upon light excitation. The term “fluorophore” refers to a fluorescent chemical compound that can re-emit light upon light excitation. Fluorophores may typically contain several combined aromatic groups, or planar and cyclic molecules with several pi bonds. The term “fluorescent dye” encompasses both fluorescent polymeric dyes and fluorescent non-polymeric dyes, including fluorescent monomeric and other traditional fluorescent dyes. The fluorescent polymer dye may be any appropriate fluorescent polymer dye.

The composition of according to the instant disclosure can be used with any polymer dye conjugate. The polymer dye conjugate can be a tandem polymer dye conjugate. The polymer dye conjugate can comprise any previously disclosed or commercially available fluorescent polymer dye. For example, the polymer dye can be any dye disclosed in Published PCT Ap. No. WO 2017/180998; U.S. Application No. 2021/0047476; U.S. Application No. 2020/0190253; U.S. Application No. 2020/0048469; U.S. Application No. 2020/0147615; U.S. Application No. 2021/0108083; U.S. Application No. 2019/0194467; U.S. Application No, 2018/0364245; U.S. Application No. 2018/0224460; U.S. Pat. Nos. 11,034,840; 11,119,107; 10,962,546; 10,920,082; 10,001,475; 10,107,818; 10,228,375; 10,844,228; 10,605,813; 10,604,657; 10,545,137B2; 10,533,092; 10,472,521; 10,240,000; 9,971,998; 9,758,625; 9,719,998; 7,214,489; 9,012,643; 8,623,332; 8,431,416; 8,354,239; 8,575,303; 8,969,509; and WO 2022/013198, each of which are incorporated by reference as if fully set forth herein in their entirety. The polymer dye can have e structure of any water-soluble fluorescent polymer dye disclosed in Published US Appl. No. 2020/0190253 A1, which is incorporated by reference as if fully set forth its entirety. The polymer dye conjugate can have the structure of any water-soluble florescent polymer dye disclosed in Published US Appl. No. 2019/0144601, which is incorporated by reference as if fully set forth herein in its entirety.

The polymer dye conjugate can be any commercially available polymer dye excitable by, for example, ultraviolet (e.g., 351 nm, 355 nm, 375 nm, 334-364 nm, 351-356 nm), violet (e.g., 405 nm, 407 nm, 414 nm, 395-425 nm), blue (e.g., 436 nm, 458 nm), blue-green (e.g., 488 n), green (e.g., 514 n, 532 nm, 541 nm, 552 nm), yellow-green (e.g., 561 nm, 563 nm), yellow (e.g., 568 nm), red (e.g., 627-640 nm, 633 nm, 637 nm, 640 nm, 647 nm), and/or near infrared lasers (e.g., 673 nm, 750 nm, 780 nm, or in a range of from 660-800 n). The polymer dye may comprise a polymer dye excitable by a violet laser. The polymer dye or polymer dye conjugate may comprise a polymer dye excitable by a violet laser at a wavelength from about 395 nm to about 425 in, for example, 405 nm, 407 nm, or 414 nm. The polymer dye or polymer dye conjugate may comprise a violet laser (405 nm)-excitable polymer dye.

In some embodiments, the polymer dye conjugate may comprise a SuperNova polymer dye (SN) (Beckman Coulter, Inc.). SuperNova polymer dyes are a new generation of polymer dyes useful for flow cytometry application. The polymer dye or polymer dye conjugate may comprise SNv428, SNv605 or SNv786, SNv428 has unique photo-physical properties leading to extremely bright conjugates when conjugated to antibodies or other binding agents. For example, SNv428 is a polymer dye optimally excited by the violet laser (e.g., 405 nm) with an excitation maximum of 414 nm, an emission peak of 428 nm, and can be detected using a 450/50 bandpass filter or equivalent. SNv428 is one of the brightest dyes excitable by the violet laser, so it is particularly suited for assessing dimly expressed markers. SuperNova polymer dye conjugated with antibodies may include anti-CD19 antibody-SNv428, anti-CD22 antibody-SNv428, anti-CD25 antibody-SNv428, and anti-CD38 antibody-SNv428 antibody-polymeric dye conjugates. SNv605 and SNv786 (Beckman Coulter, Inc.) are tandem polymer dyes, derived from the core SNv428. Both share the same absorbance characteristics, with maximum excitation at 414 nm. With SNv605 and SNv786 having emission peaks at 605 nm and 786 nm, respectively, they are optimally detected using the 610/20 and 780/60 nm bandpass filters of the flow cytometer. SNv605 and SNv786 may be conjugated, for example, with anti-CD19 antibody, anti-CD22 antibody, anti-CD25 antibody, and anti-CD38 antibody.

The polymer dye conjugate may comprise a polymer dye excitable by an ultra-violet (“UV”) laser. The polymer dye or polymer dye conjugate may comprise a polymer dye excitable by a UV laser at a wavelength of 320 nm to 380 am, 340 nm to 360 nm, 345 nm to 356 nm, or less than or equal to 380 nm but greater than or equal to 320 nm. The polymer dye or polymer dye conjugate may comprise a UV-excitable polymer dye. The UV-excitable polymer dye or polymer dye conjugate may emit light typically at a wavelength of 380 nm to 1000 nm, 380 nm to 800 nm, 380 nm to 430 nm, 406 nm to 415 nm, or less than or equal to 430 nm but greater than or equal to 380 nm.

The polymer dye conjugate can comprise a Brilliant Violet™ dye (BioLegend®/Sirigen Group Ltd.), such as Brilliant Violet 421™ (excitation max. 405 nm, emission max. 421 nm, 450/50 filter), Brilliant Violet 510™ (excitation max 405 nm, emission max 510 mm, 510/50 filter), Brilliant Violet 570™ (excitation max 405 nm, emission max 570 inn, 585/42 filter), Brilliant Violet 605™ (excitation max 405 nm, emission max 603 nm, 610/20 filter), Brilliant Violet 650™ (excitation max 405 rim, emission max 645 nm, 660/20 filter), Brilliant Violet 711™ (excitation max 405 nm, emission max 711 nm, 710/50 filter), Brilliant Violet 750™ (excitation max 405 nm, emission max 750 nm, 780/60 filter), Brilliant Violet 785™ (excitation max 405 nm, emission max 785 nm, 780/60 filter). The polymer dye or polymer dye conjugate may comprise a Spark Violet™ 538 (BioLegend, Inc.)(excitation max 405 mm, emission max 538 nm).

The polymer dye conjugate may comprise a Super Bright polymer dye (Invitrogen, ThermoFisher Scientific). Super Bright dyes may be excited by the violet laser (405 nm). The Super Bright dye may be Super Bright 436 (excitation max 414 nm, emission max 436 nm, 450/50 bandpass filter), Super Bright 600 (emission max 600 nm, 610/20 bandpass filter), Super Bright 645 (emission max 645 nm, 660/20 bandpass filter), or Super Bright 702 (emission max 702 un, 710/50 bandpass filter).

The polymer dye conjugate may comprise a BD Horizon Brilliant™ Violet (“BV”) polymer dye (Becton, Dickinson and Co., IBD Life Sciences). The polymer dye may comprise a BD Horizon Brilliant™ BV421 (450/40 or 431/28 filter), BV480 (525/40 filter), BV510 (525/40 filter), BV605 (610/20 filter), BV650 (660/20 filter), BV711 (710/50 filter), BV786 (786/60 filter) polymer dye.

### Methods

A method is provided for reducing or eliminating non-specific binding, such as polymer-polymer interactions of at least one polymer dye conjugate, or at least two polymer dye conjugates in a biological sample, such as a blood sample, the method comprising: contacting the at least one dye conjugate with at least one UV-polymer dye and a nonionic surfactant before, during, or after the polymer dye conjugate is contacted with a biological sample, the contacting resulting in decreased non-specific binding, such as reduced polymer-polymer interactions, of the at least one or at least two polymer dye conjugates in the biological sample.

In some embodiments, the present disclosure provides a method for reducing or eliminating non-specific binding, such as polymer-polymer interactions, of at least one or at least two polymer dye conjugates in a biological sample, the method comprising: contacting that at least one polymer dye conjugate with a UV-absorbing polymer dye and a nonionic surfactant before, during, or after the dye conjugate is contacted with the biological sample, the contacting resulting in decreased non-specific binding between polymer dye conjugates in the sample. In some embodiments, the present disclosure provides a method for reducing or eliminating polymer-polymer interaction between at least one or at least two polymer dye conjugates in a multi-color panel, the method comprising: contacting the at least one or the at least two polymer dye conjugate(s) with at least one UV-absorbing polymer dye and/or nonionic surfactant before, during or after the polymer dye conjugate(s) is contacted with a blood sample, the contacting resulting in decreased non-specific binding of the at least one or the at least two polymer dye conjugate(s) in the biological sample. The compositions and methods of the disclosure reduce or eliminate non-specific binding of polymer dye conjugates in a blood sample.

### EXAMPLES

Various embodiments of the present disclosure can be better understood by reference to the following Examples which are offered by way of illustration. The present disclosure is not limited to the Examples given herein.

### Example 1. Initial Screening

The initial focus was to identify one or more modifying units which upon reacting with a monomer, such as a DHP monomer, could shift polymer dye absorbance maximum close to 355 nm and minimize absorption at 405 un. A general reaction scheme for preparation of the polymers is shown in Scheme 1.

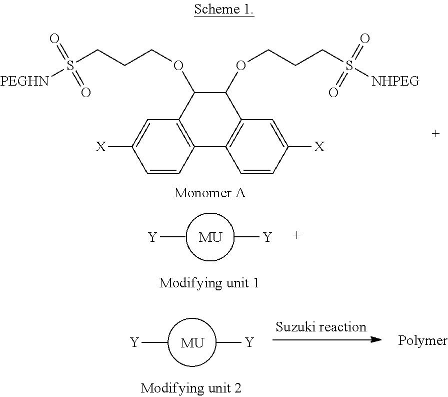

Initial screening of the monomers and modifying units was carried out using test polymerization reactions.

Modifying units which produced polymers with good absorption in the 355 nm and minimum excitation at 405 nm, were chosen for the polymerization in larger scale.

Test polymerization reactions using a DHP monomer and one or two modifying units (in 1:0.5:0.5 ratio) were carried out. In Table 1B, DHP monomer units wherein f=11-40 were evaluated. Both absorbance and emission maxima were monitored. Table 1B entry 1 showed an absorption maximum at 348 nm and an emission maximum at 408 nm. In addition, the absorption spectrum was relatively sharp and therefore the cross-talk at 405 nm was minimized (the cross-talk was 2.1). The polymer showed excellent photophysical properties in terms of brightness, low cross-talk and high conjugation yields. Test polymerizations of DHP monomers using one and two modifying units were carried out as shown in Table 1B entries 1-16. Based on these results it was decided to progress with the DHP monomer along with difluoro and trifluoro-based modifying units as the components of the target polymer (as shown in entry 1). Other preferred target polymers comprise the polymerization products of Table 1B, entries 2, 3, 4, 5, 6, 7, 12, or 15. The variable “V” in Table 1B is an integer from 11-40. The variable “p” in Table 1B is 18.

TABLE 1B
Test polymerizations.

| | | | | Abs | Em |
|---|---|---|---|---|---|
| No | DHP used | Modifying unit 1 | Modifying unit 2 | nm | nm |
| 1 | 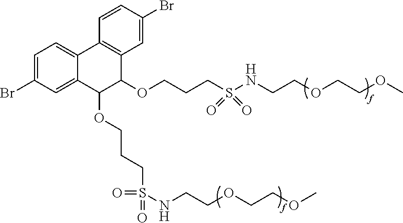 | 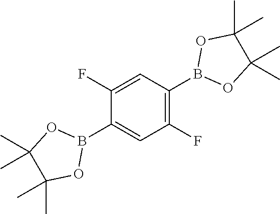 | 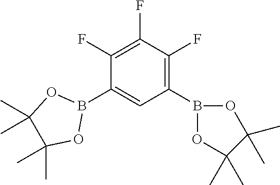 | 348 | 408 |
| 2 | 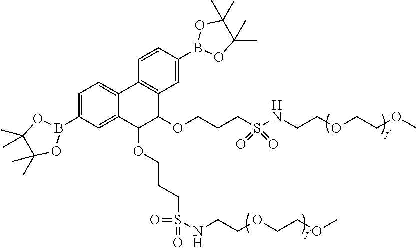 | 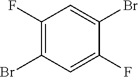 | 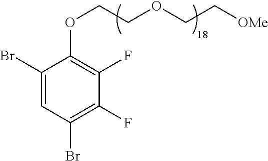 | 356 | 413 |
| 3 | 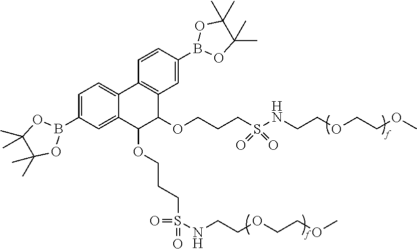 | 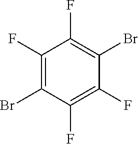 [SMILES: Fc1c(F)c(Br)c(F)c(F)c1Br] | 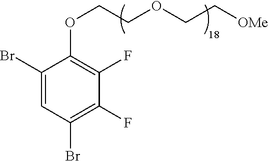 | 340 | 413 |
| 4 | 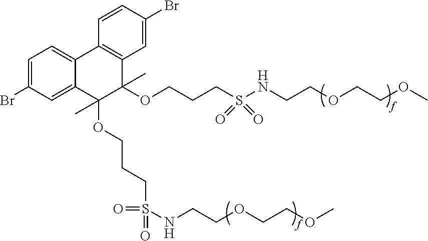 | 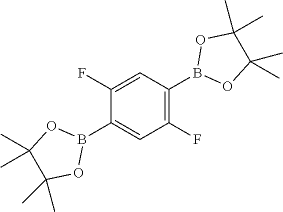 | 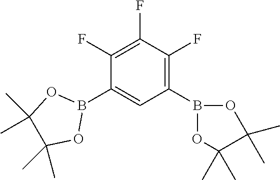 | 352 | 409 |
| 5 | 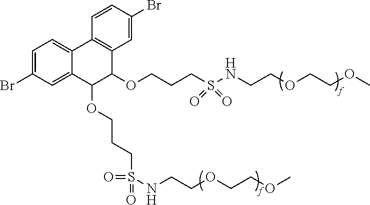 | 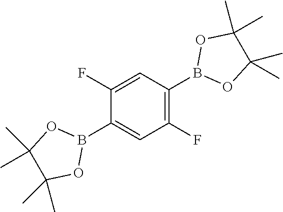 | n.a. | 366 | 412 |
| 6 | 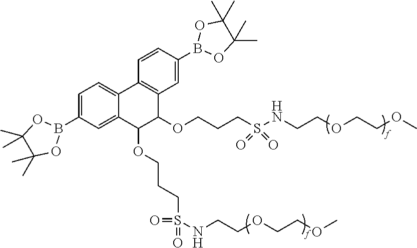 | 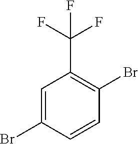 [SMILES: FC(F)(F)c1cc(Br)ccc1Br] | n.a. | 354 | 412 |
| 7 | 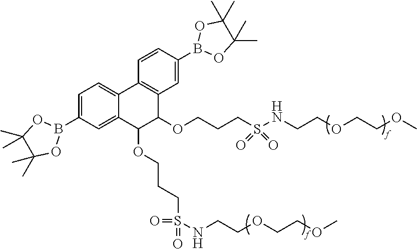 | 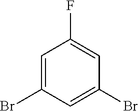 | n.a. | 346 | 415 |
| 8 | 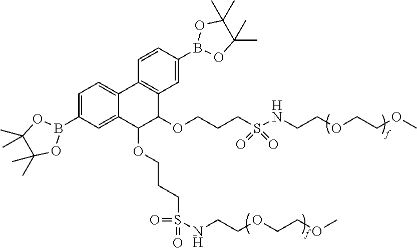 | 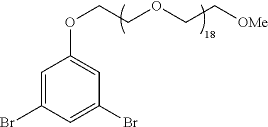 | n.a. | 343 | 415 |
| 9 | 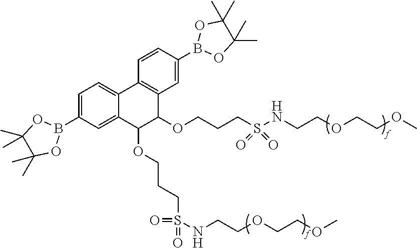 | 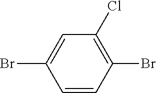 | n.a. | 369 | 416 |
| 10 | 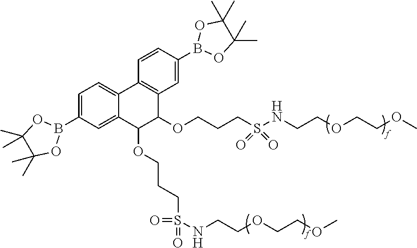 | 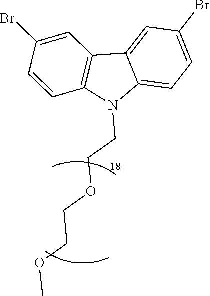 | n.a. | 354 | 440 |
| 11 | 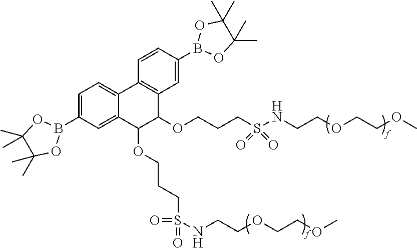 | 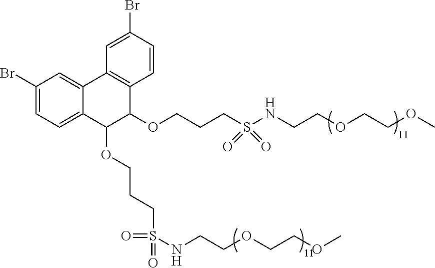 | n.a. | 335 | 407 |
| 12 | 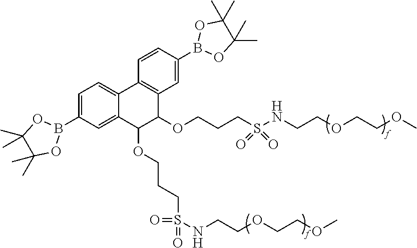 | 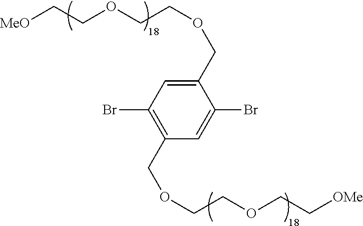 | n.a. | 352 | 413 |
| 13 | 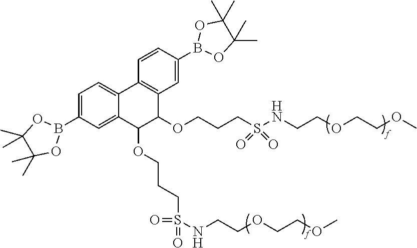 | 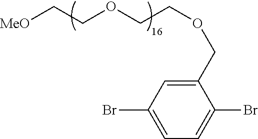 | n.a. | 371 | 418 |
| 14 | 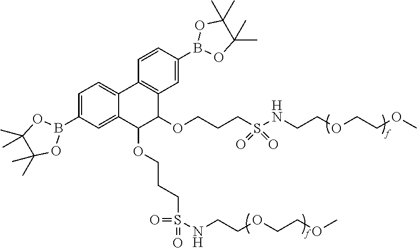 | 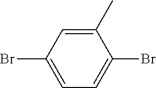 | n.a. | 367 | 417 |
| 15 | 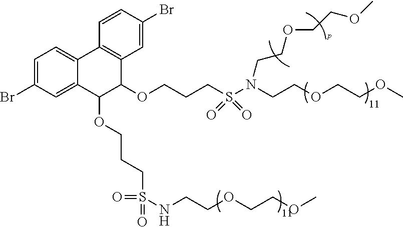 | 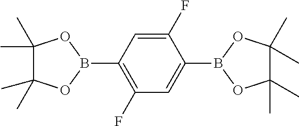 | n.a. | 360 | 404 |
| 16 | 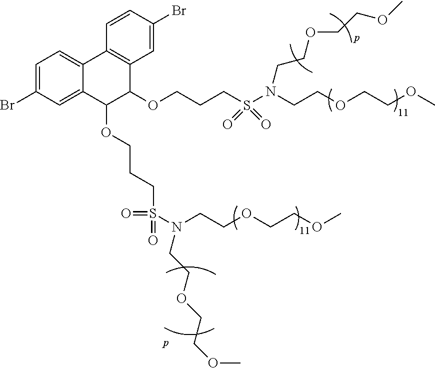 | 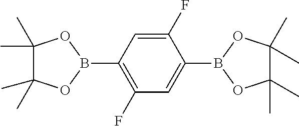 | n.a. | 364 | 405 |

n.a. = not applicable

### Example 2. Synthesis of Capped Polymers

Method 1: In a round bottom flask both the modifying units and the DHP monomer were taken in (DMF-water) mixture and purged with nitrogen for 10 minutes. Under nitrogen about 20 equivalents of CsF and 10% of Pd(OAc)₂ were mixed and heated at 80° C. Polymerization was monitored using UV-Vis spectroscopy and SEC chromatography. Later to the reaction mixture, a capping agent (selected from G1) containing appropriate functional group was added and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system.

Method 2: In a round bottom flask both the modifying units and the DHP monomer (1:1) were taken and dissolved in THF-water (4:1) mixture containing 10 equivalents of K₂CO₃ and 3% Pd(PPh₃)₄. The reaction mixture was put on a Schlenk line and was degassed with three freeze-pump-thaw cycles and then heated to 80° C. under nitrogen with vigorous stirring for 18 hours. Later to the reaction mixture, a capping agent (selected from G1) containing appropriate functional group was added via a cannula under excess nitrogen pressure and 3 hours later the second capping agent (selected from G2) added. After the reaction the crude reaction mixture was evaporated off and passed through a gel filtration column to remove small organic molecules and low MW oligomers. Later the crude polymer passed through a Tangential flow filtration system.

The polymer was further characterized using NMR (quality of polymer), GPC (Mw, Mn and P1)) and spectroscopy (molar extinction coefficient, quantum yields, brightness) and capping efficiency.

The polymer had the following structure X (“f” is an integer from 0 to 50, or 11 to 40, and G¹ and G² are as described herein):

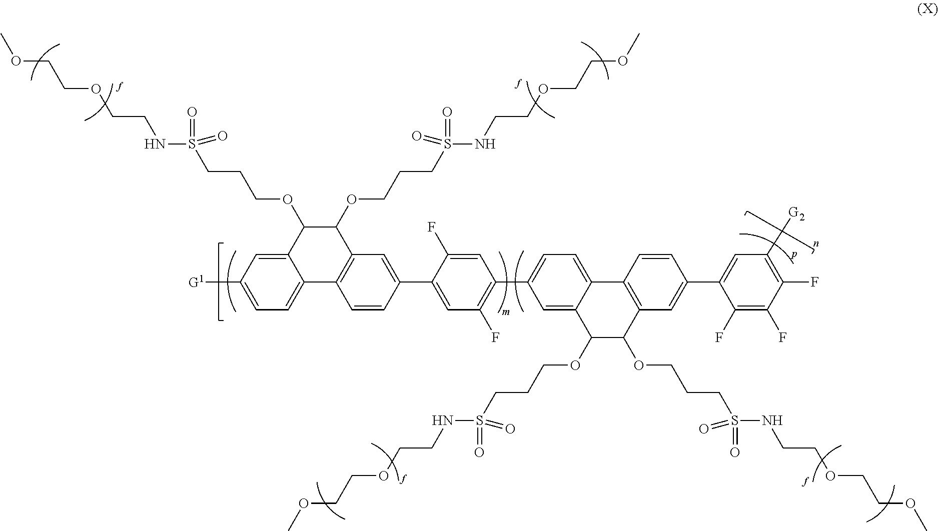

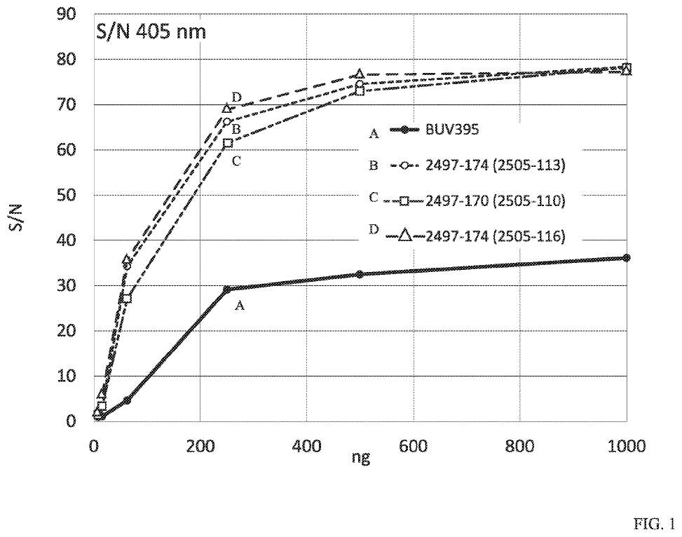

FIG. 1 illustrates a comparison of the signal-to-noise ratio at 405 nm channel of 3 different lots (B, C, D) UV-absorbing polymer conjugated with CD4 antibody compared to BUV395-CD4 conjugate (A) (Becton Dickinson Biosciences). Laser excitation was at 355 nm.

FIG. 1 shows that the conjugated polymer on a 355 nm laser flow cytometer had more than 2× brightness at 405 nm channel compared to BUV395 CD4 conjugate A. Table 2 shows absorbance of BUV395-CD4 conjugate and the polymerCD4 conjugates at 375 nm and 355 un. Later this polymer CD4 conjugate was analyzed on a 375 nm laser flow cytometer and it had a brightness of ˜5.5× at 405 nm compared to BUV395 CD4 conjugates. This result makes sense because, as shown in Table 2, the polymer CD4 conjugates had ˜75% absorbance at 375 nm compared with the absorbance at 355 nm while BUV395 CD4 conjugate has only a ˜5% difference in absorbance at 375 nm compared to 355 un. Thus, the inventive UV-absorbing polymerCD4 conjugate showed better performance on the 375 nm laser system than that of comparative BUV395 CD4 conjugate. This can be an advantage for the polymer in both 355 and 375 nm laser flow systems.

TABLE 2
Flow performance of CD4 conjugates at 355 and 375 nm.

| | Stain index measured | Stain index measured at |
|---|---|---|
| | at 375 nm Laser | 355 nm Laser |
| Dye | CytoFlex S | CytoFlex LX |
| Comparative BUV395 CD4 | 42.1 | 42.5 |
| conjugate | | |
| Inventive UV Polymer CD4 | 179.6 | 87.1 |
| conjugate | | |

### Example 3. Polymer-Tandem Dyes

In this Example, a polymer-tandem dye bearing an acceptor dye was made, following the general methods given in Example 2, with the polymer having the structure shown below (“f” is an integer from 0 to 50, and G¹ and G² are as described herein), which was then conjugated to CD4 antibody:

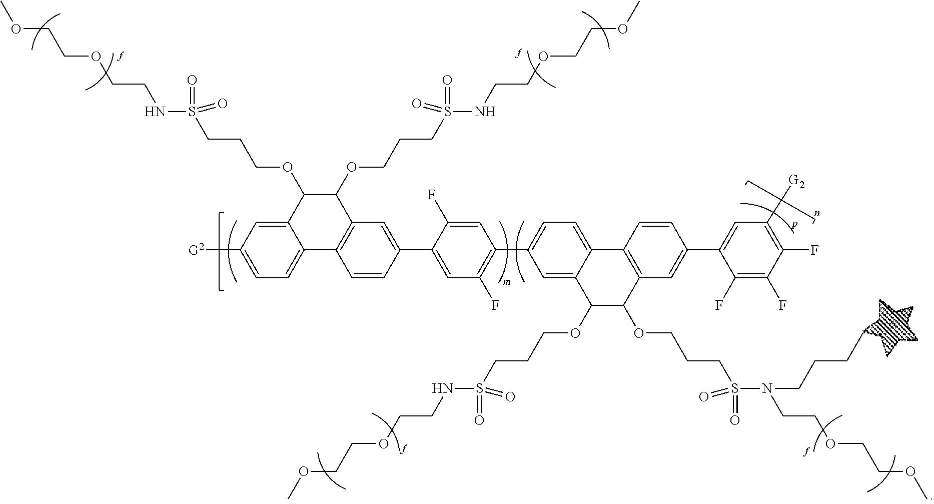

To form the polymer-tandem, dye, a polymer from Example 2 was used. A reaction scheme for the syn thesis is shown in Scheme 2.

Scheme 2. Synthesis of polymer-tandem dye.

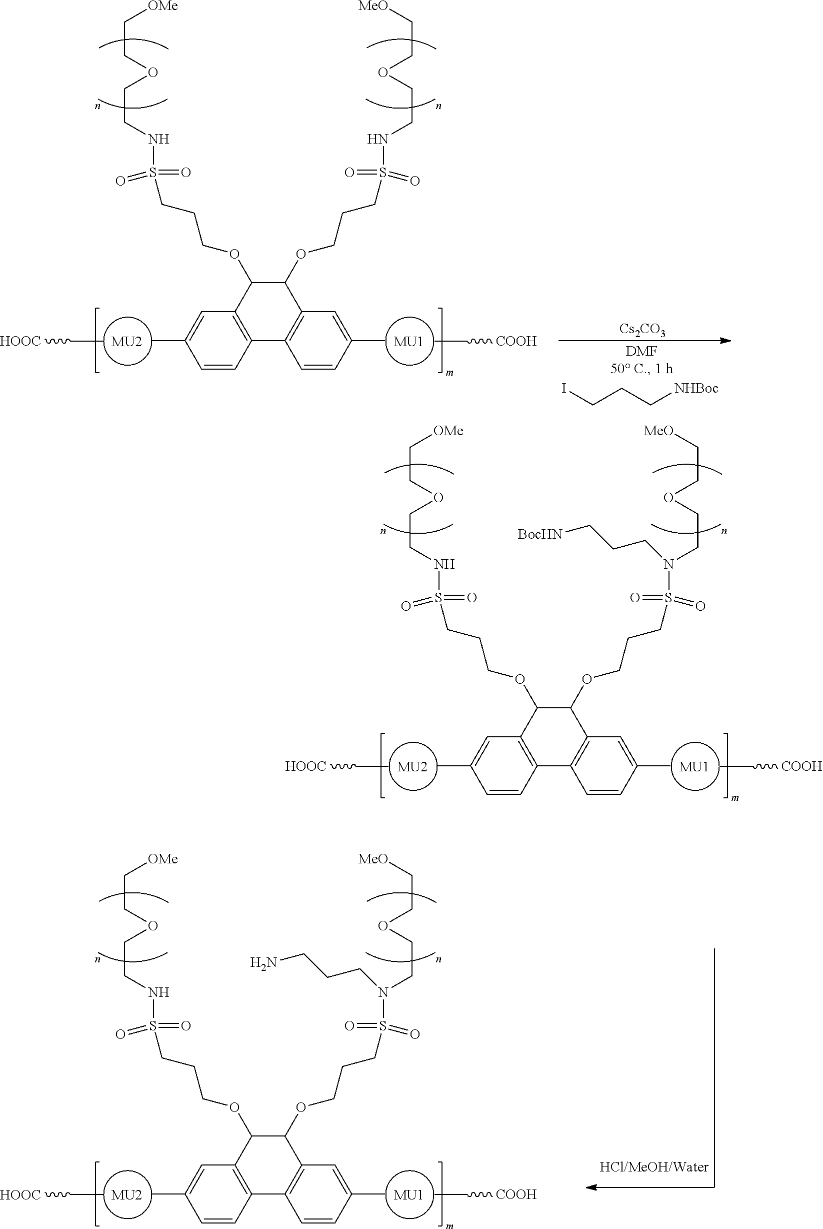

Procedure to synthesize NHBoc UV polymer: Under nitrogen atmosphere, the polymer solution was transferred to a 10 ml reaction flask containing cesium carbonate (100 eq.). tert-Butyl-3-iodopropyl-carbamate solution was diluted from a stock solution (10 mg/ml in anhydrous DMF), and 10 eq. was added to the polymer mixture. The sealed reaction flask was heated to 50° C., and the reaction was continued for 1 h under stirring at 500 rpm. The reaction mixture was cooled to RT and the DMF was evaporated in a rotary evaporator under high vacuum. The crude reaction mixture was diluted with chloroform (25 mL) and washed with 15% w/v brine solution (25 mL). The organic layer was collected in a 250-mL conical flask, additional chloroform (12 mL) was added, and the mixture was washed three times with 30% w/v brine solution (10 mL). The organic fraction was dried by adding 20 g anhydrous sodium sulfate and then filtered through Whatmnan Paper 2 into a 150 mL flat bottom flask. The filtered sodium sulfate was washed twice with chloroform (15 mL) to recover the remaining polymer dye and filtered into the same flat bottom flask. The chloroform was evaporated in a rotary evaporator at 45° C. and 150-200 rpm. Residual DMF was removed under a high vacuum pump at 50° C. for 30-40 minutes. The dried polymer was washed with diethyl ether (2×2 mL) and sonicated for two minutes to eliminate the unreacted tert-butyl-3-iodopropyl-carbamate. After drying the polymer under high vacuum for 5 min, the yield of the polymer was calculated with respect to the initial polymer amount. The dried polymer product was characterized using 1H NMR; proton signals at 1.4 ppm indicate the existence of NH-Boc moieties in polymer.

Procedure to synthesize NH2 UV polymer: 50 mg of the NHBoc polymer prepared as described above was added to a 20 mL round-bottom flask and dissolved in 1 mL methanol and 1 mL water by vortexing for 5 minutes & sonication for 5 minutes. To the resulting solution was added 12 M HCl (2 mL) and the mixture was allowed to react for 2 h at room temperature. The reaction mixture was then transferred to a small beaker, the pH was adjusted to 9-10 using 15% w/v K₂CO₃ solution, and stirred for an additional 15 minutes. The polymer was extracted with 25 mL chloroform in a 100 mL separation funnel, the organic layer was and collected in a conical flask. Brine solution (15% w/v) was added to the aqueous layer and additional portions of chloroform were used to recover remaining polymer. The extraction process was monitored with a UV lamp.

The organic layer was dried using ˜40 g anhydrous sodium sulfate and filtered through Whatman filter paper 2 into a 250 mL flat bottom flask. Additional chloroform washes (2×20 mL) were used to recover remaining polymer f-om the filtered sodium sulfate. The combined chloroform layer was evaporated in a rotary evaporator at about 40° C. After complete solvent evaporation, the solid was dissolved again chloroform (10 nL) and centrifuged at 3000 rpm for 5 min to remove the salt impurities in a 15 mL Falcon tube. The supernatant was decanted in 20 mL vial and concentrated on a rotary evaporator and dried under high vacuum. The yield of the deprotected amine-functionalized polymer was calculated with respect to the protected polymer amount.

To form the polymer-tandem dye, 10 mg of polymer was weighed in a glass vial and dissolved in 200 μL of anhydrous DMSO. To ensure the polymer was completely dissolved, a combination of vortex, sonication, and incubation at 50° C. water bath in about 10-15 min were applied. To this, 200 μL acetonitrile and 20 μL diisopropylethylamine were added. A 10 mg/mL (w/v) solution of an acceptor dye NHS ester (e.g., an acceptor dye with an emission centered around 700-800 nm) was prepared in anhydrous DMSO, and 8 equivalents of dye were added to the polymer solution. The mixture was stirred for two hours at room temperature, protected from light, resulting in a product containing an average of 2-3 dyes per polymer chain. Products containing 1-6 dyes per polymer chain can be prepared by adjusting the amount of acceptor dye used in the reaction. The polymer-tandem due was conjugated with CD4 antibody.

Absorption and emission measurements of the purified polymer-CD4 conjugate were carried out. Acceptor to donor ratio was calculated as 1.7. Fluorescence Resonance Energy Transfer (FRET) was studied by exciting the polymer at 355 nm which resulted in an emission from the acceptor dye due to FRET along with >90% quenching of the donor emission. When it was excited at 405 nm, there was hardly any fluorescence observed from both polymer and the acceptor dye. This shows that the current backbone can be modified using standardized techniques, and the efficient energy transfer can be obtained by finding a suitable acceptor dye.

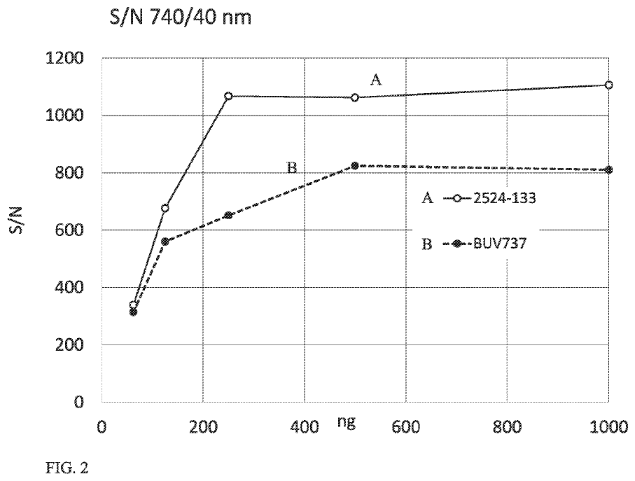

FIG. 2 illustrates the signal-to-noise ratio at the S/N 740/40 nm channel (with laser excitation at 355) of the comparative CD4 conjugate of BD Horizon BUV737 versus the inventive UV polymer-Dy704 tandem-CD4 conjugated polymer-tandem dye formed in this Example,

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Example 4: Procedure for Surface Staining with Concomitant Fixation Buffer in Sample Preparation for Flow Cytometry

In this procedure, a staining buffer according to the disclosure is added into the test tube before addition of dye conjugates in order to avoid any possible non-specific interactions that may occur between the dye conjugates over time. Fixation is a stage which enables leukocytic preparations to be stored for several hours without deterioration, after staining with a fluorescent antibody. Lysing solution may be used for lysis of red blood cells in the preparation of biological samples for flow cytometry.
1. Extemporaneously prepare the “Fix-and-Lyse” mixture by adding 25 μL of undiluted IOTest 3 10× Fixative Solution (A07800, Beckman Coulter, Inc.) to 1 mL of VersaLyse™ lysing solution (A09777, Beckman Coulter, Inc.). Prepare a sufficient volume of the “Fix- and Lyse” mixture depending on the number of biological test samples to be lysed (1 uL of mixture per tube).
2. In each test tube, add 10 μL or 20 μL of the staining buffer according to the disclosure. 3. Add the appropriate volumes of dye conjugates. Vortex the tubes gently.
4. Add 100 μL of the test sample to each tube. Vortex the tubes gently.
5. Incubate for 15 to 20 minutes at room temperature (18-25° C.), protected from light. Then perform lysis of the red cells:
6. Add 1 ml of the “Fix-and-Lyse” mixture prepared extemporaneously and vortex immediately for one second.
7. Incubate for 10 minutes at room temperature, protected from light.
8. Centrifuge for 5 minutes at 150×g at room temperature.
9. Remove the supernatant by aspiration.
10. Resuspend the cell pellet using 3 mL of PBS.
11. Centrifuge for 5 minutes at 150×g at room temperature.
12. Remove the supernatant by aspiration.
13. Resuspend the cell pellet using 0.5 mL of PBS plus 0.1% Formaldehyde (A 0.1% formaldehyde PBS can be obtained by diluting 12.5 μL of the IOTest 3 Fixative Solution (see Catalog for PN) at its 10× concentration in 1 mL of PBS).

These preparations may be kept 24 hours between 2 and 8° C. and protected from light before analysis by flow cytometry.

### Example 5. Staining Buffer Composition Component Selection to Avoid Non-Specific Interaction with Cells

A staining buffer composition was developed for use with multi-color panels comprising one or more polymer dye conjugates for use in staining a biological sample in flow cytometry. A first goal was to select composition components that would avoid non-specific interaction with cells in a biological sample.

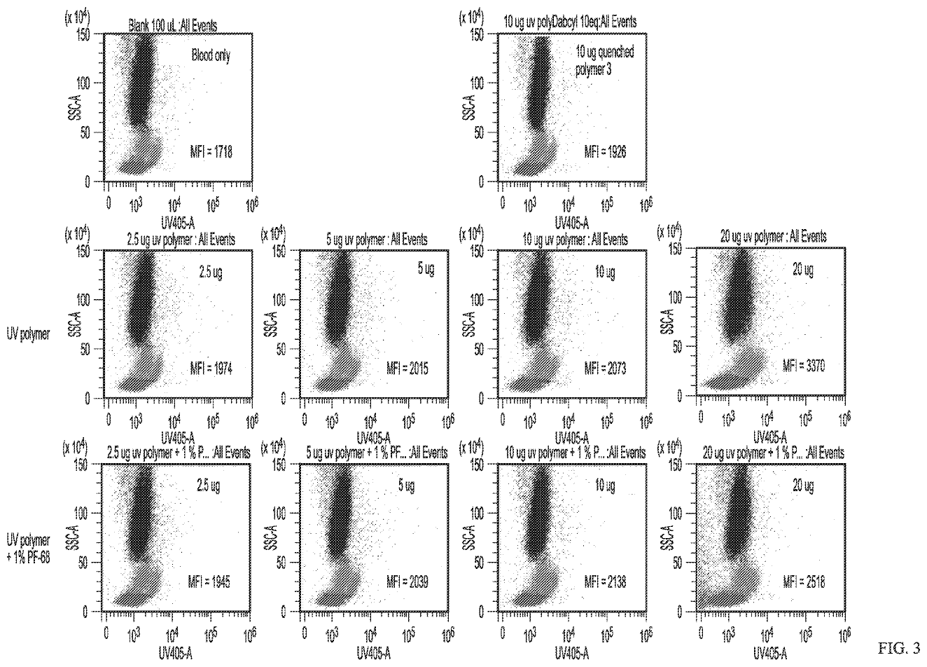

Various concentrations of UV-absorbing polymer according to the disclosure with or without 1% PF-68 were added to human whole blood. Red blood cells were lysed, white blood cells were washed 2 times and ran in CytoFlex LX flow cytometer. FCA dot plots are shown in FIG. 3. Upper panel shows blood only without additives, middle four panels show addition of UV-absorbing polymer according to the disclosure at (left to right), 2.5 ug/test, 5 ug/test, 10 ug/test, and 20 ug/test, lower four panels show addition of UV-absorbing polymer according to the disclosure at (left to right), 2.5 ug, 5 ug, 10 ug, and 20 rug with 1% PF-68. Upper right panel shows addition of 10 ug quenched polymer 3. MFI values of monocytes indicating that UV polymer (2.5 ug-20 ug/test) with or without 1% PF-68 did not show strong non-specific binding to cells as compared with control cells (Blood only panel).

An exemplary staining buffer composition provides 5-20 ug/test UV polymer dye according to the disclosure, 0.1-2%/test PF-68 in PBA buffer (PBS/BSA/NaN₃).

A representative composition for adding to a multi-color panel comprising polymer dye conjugates for staining a biological sample, e.g., prior to FCA analysis, is shown in Table 4. One exemplary staining buffer composition comprises 0.5 mg/mL UV polymer, 7% PF-68, 2 mg/mL BSA, 0.02% NaN₃ in PBS buffer. The UV polymer may be any UV polymer according to the present disclosure. The UV polymer may be a tandem UV polymer comprising one or more acceptor dyes. The UV polymer may be a quenched UV polymer comprising one or more quenching moieties.

A method of making a staining buffer composition was developed.

Stock solutions were prepared as follows. UV polymer: Weigh 1.3 mg polymer then add 130 uL DMSO to dissolve the polymer by vortexing to make a 10 mg/mL UV polymer stock solution. PF-68: A commercial solution of 10% PF-68 is used as received. BSA: Weigh 20 mg BSA then add 1 mL buffer-PBS to make a 20 mg/mL stock solution. NaN₃: Weigh 10 mg NaN₃ then add 1 mL buffer-PBS to make a 1% stock solution.

The staining buffer composition was formulated using the stock solutions above as shown in Table 3.

TABLE 3
Staining Buffer Preparation

| Component | Stock Conc. | Solvent | Final Amount | Volume (μL) |
|---|---|---|---|---|
| UV polymer | 10 mg/mL | DMSO | 0.5 mg/mL | 100 |
| PF-68 | 10% | N/A | 7% | 1400 |
| BSA | 20 mg/mL | PBS | 2 mg/mL | 200 |
| NaN₃ | 1% | PBS | 0.02% | 40 |
| PBS | 1X | N/A | N/A | 260 |
| Total | | | | 2000 |

For example, the staining buffer composition of Table 4 may be added to the polymer dye conjugates before staining cells. For example, 20 uL of the staining buffer composition may be added to a test tube, followed by the polymer dye conjugates prior to adding biological sample.

### Example 6. Performance of Staining Buffer Composition with Two Different UV Polymer Dye Conjugates

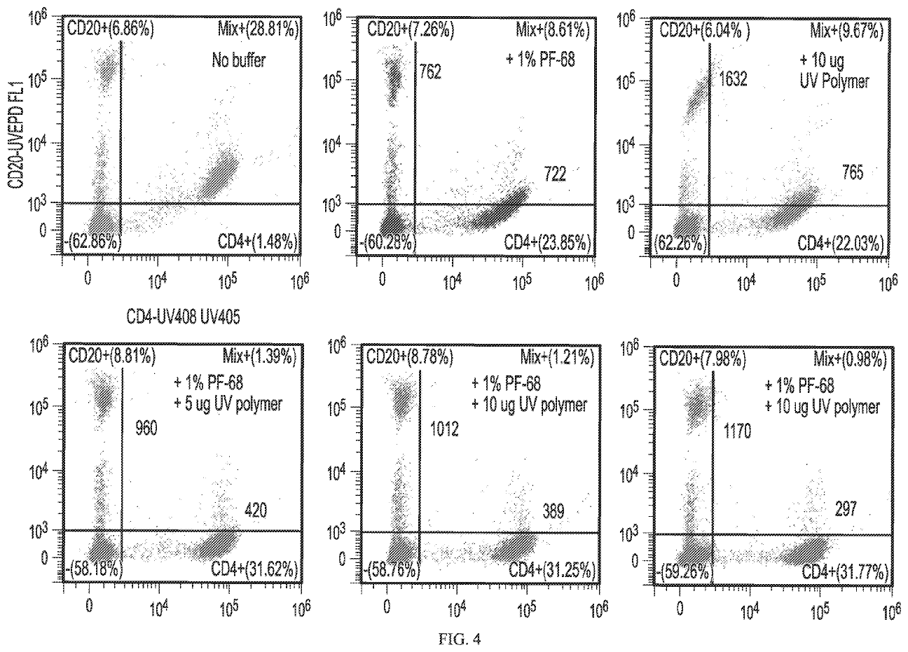

Human whole blood was stained with two polymer dye conjugates: CD20-UV excitable polymer dye (UVEPD) and SN uv408-CD4, both according to the disclosure (Beckman Coulter Life Sciences), in the presence of various concentrations of UV polymer, with/without additives. FCA dot plots of stained cells with the two polymer dye conjugates and various additives are shown in FIG. 4. Upper left panel shows stained cells without additives; upper middle panel shows stained cells with 1% PF-68; upper right panel shows stained cells with UV polymer according to the disclosure (10 ug/test); lower left panel shows stained cells with 5 ug UV polymer and 1% PF-68; lower middle panel shows stained cells with 10 ug/test UN polymer and 1% PF-68; and lower right panel shows stained cells with 20 ug UV polymer and 1% PF-68. In this example, stained cells in the presence of combined UV polymer (5-20 μg/test)+1% PF-68 (lower 3 panels) showed less spillover than the controls including no buffer added sample (upper left panel), 1% PF-68 (upper middle panel), and LV polymer alone (upper right panel). The values in each panel indicate the MFI values, Example 7. Performance of Staining Buffer Composition with Two Different Violet Polymer Dye Conjugates

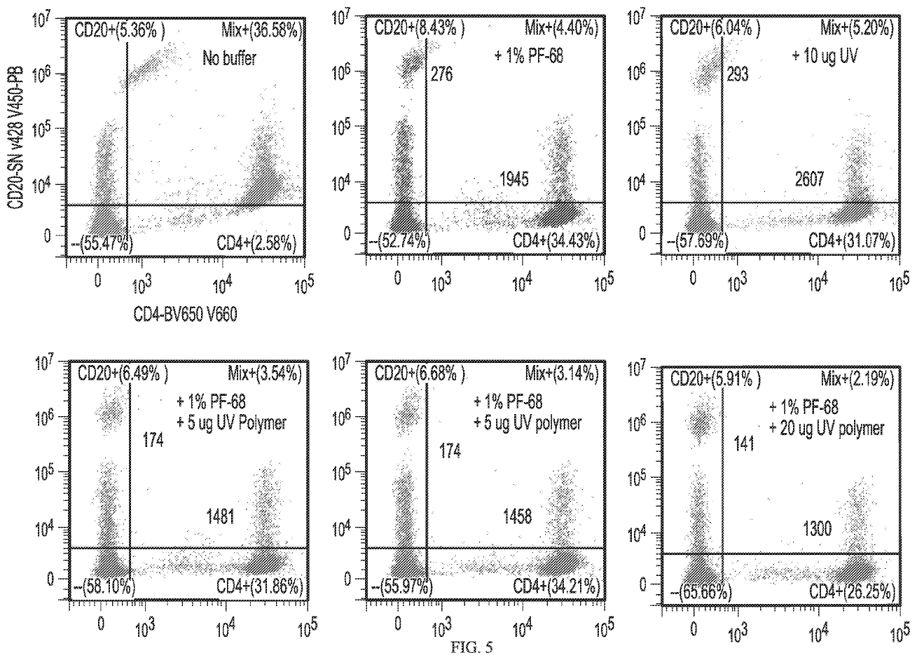

Human whole blood was stained and lysed with CD20-SN v428 (Beckman Coulter Life Sciences) and CD4-BV650 (BD Biosciences) polymer dye conjugates with/without additives and/or UV polymer. FCA dot plots are shown in FIG. 5. Upper left panel shows stained cells in PBS without additives; upper middle panel shows stained cells with 1% PF-68; upper right panel shows stained cells with UV polymer according to the disclosure (10 ug/test); lower left panel shows stained cells with 5 ug/test UV polymer and 1% PF-68; lower middle panel shows stained cells with 10 ug/test UV polymer and 1% PF-68; and lower right panel shows stained cells with 20 ug/test UV polymer and 1% PF-68.

In this example, stained cells in the presence of combined UV polymer (5-20 μg/test)+1% PF-68 (lower three panels) showed better separation than that of the controls including no buffer added sample (upper left), 1% PF-68 (upper middle), and UV polymer alone (upper right). The values in each panel indicate the MFI values.

### Example 8. Performance of Staining Buffer Composition with UV Polymer and Various Concentrations of Nonionic Surfactant

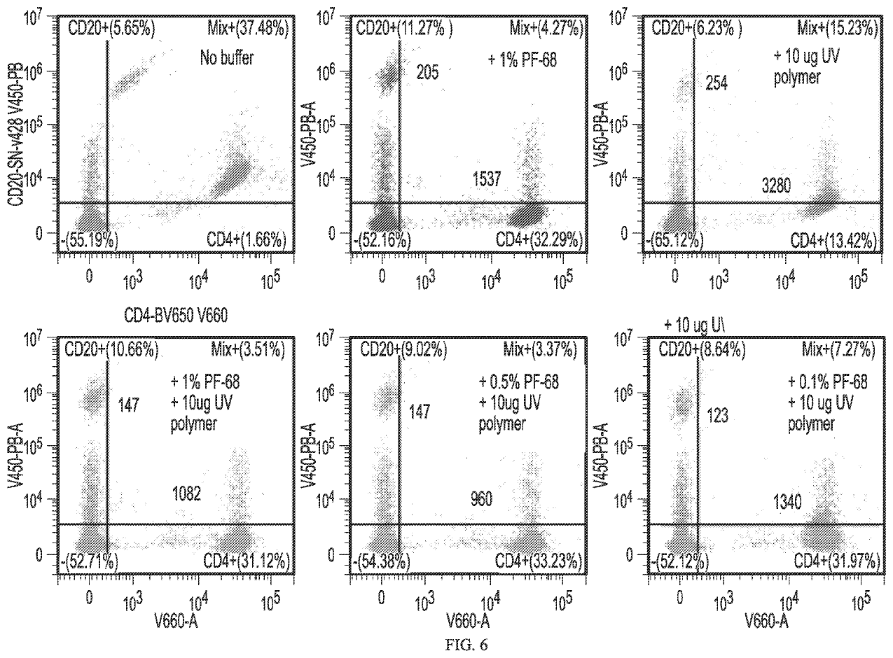

In this example, the effect of different concentrations of nonionic surfactant (0.1-1% PF-68) was investigated in test staining buffer. Human whole blood was stained with CD20-SN v428 (Beckman Coulter Life Sciences) and CD4-BV650 (BD Biosciences). FCA dot plots of stained cells are shown in FIG. 6. Upper right panel shows stained cells with UV polymer (10 ug); middle center panel shows stained cells in PBS with 1% PF-68; upper left panel shows a no buffer added sample; lower left panel shows stained cells with UV polymer and 1% PF-68; lower middle panel shows stained cells with UV polymer and 0.5% PF-68; lower right panel shows stained cells with UV polymer and 0.1% PF-68. In this example, stained cells in the presence of combined UV polymer—PF-68 in different concentrations (0.1-1%) (lower left, lower middle and lower right panels) showed better separation than that of the controls including no buffer added sample (upper left panel), 1% PF-68 (upper middle panel), and UV polymer alone (10 ug/test; upper right panel), The values in each panel indicate the MF values.

### Example 9. Flow Cytometry Performance of UV Polymer and Quenched UV Polymer Staining Buffer Compositions in Mixtures of Two Different Polymer Dye Conjugates

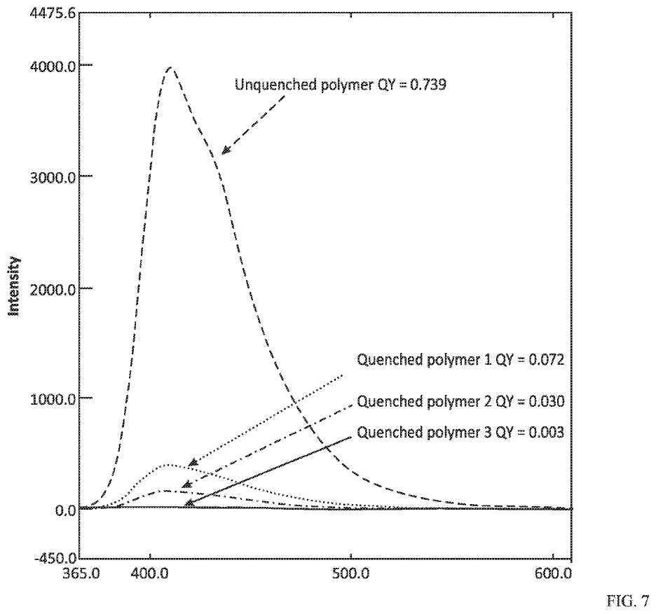

Three different quenched UV polymers (Quenched Polymers 1-3) according to the disclosure were prepared from a UV-absorbing polymer and Dabcyl dye quenching moieties. The ratio of quencher to polymer (D/P) was determined to be 2.5, 5, or 10 for the three Quenched UV Polymers. The intensity of the emission spectra of the quenched Polymers following excitation at 355 nm is shown in FIG. 7. The emission spectra from 365-600 nm is shown. The quantum yield (QY) at 405 nm of the quenched polymer 1 (D/P=2.5), quenched polymer 2 (D/P=5.0), and quenched polymer 3 (D/P=10) are 0.072, 0.030, and 0.003, respectively. The quantum yield of the unquenched polymer is 0.739.

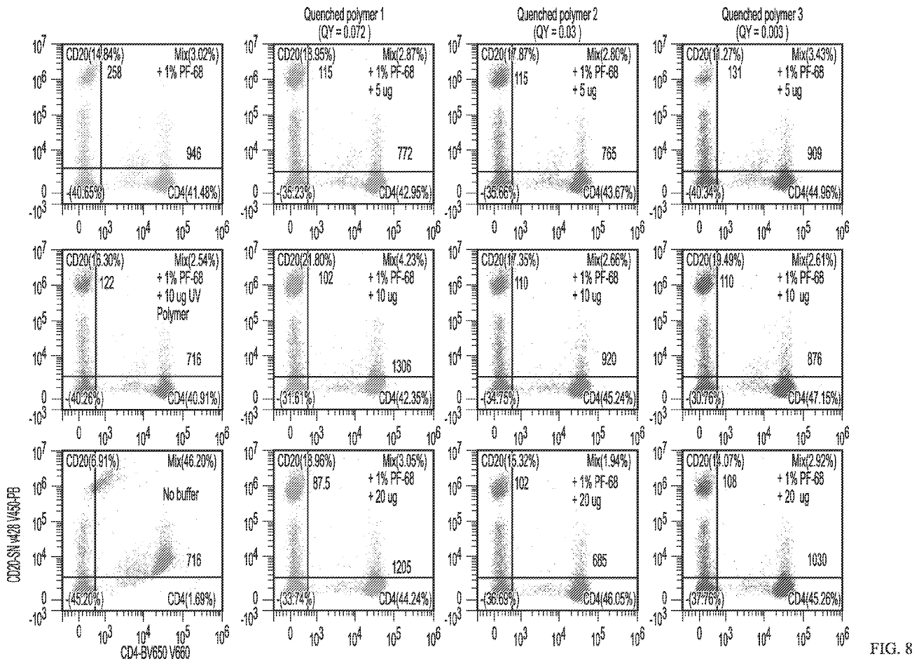

The three quenched polymers (Quenched polymer 1, Quenched polymer 2 and Quenched polymer 3) having D/P=2.5, 5, and 10 were each employed at 5 ug, 10 ug and 20 ug per test as staining buffer additives with 1% PF-68 in a mixture of two commercially available polymer dye conjugates CD20-SN v428 (Beckman Coulter Life Sciences) and CD4-BV650 (BD Biosciences) with whole blood cells. FCA dot plots of stained and lysed cells in a mixture of CD20-SN v428- and CD4-BV650 are shown in FIG. 8 without additives (lower left panel), control 1% PF-68 (upper left panel), comparative 10 ug UV polymer dye with 1% PF-68 (middle left panel), and Quenched polymers 1, 2 and 3 at 5 ug, 10 ug or 20 ug/test with 1% PF-68 (second column panels, third column panels and right column panels) stained cells in the presence of test staining buffer compositions including combined UV polymer (10 μg/test)+1% PF-68 (middle left panel) or stained cells in the presence of the quenched polymers 1, 2, and 3 at 5 ug, 10 ug or 20 ug/test, with 1% PF-68 (right three columns, upper, middle, lower panels, respectively) each showed better separation than control without additives (lower left panel). The values in each panel indicate the MFI values.

### Example 10. Flow Cytometry Performance of Nonionic Surfactant Alone in Mixtures of Two Different Polymer Dye Conjugates

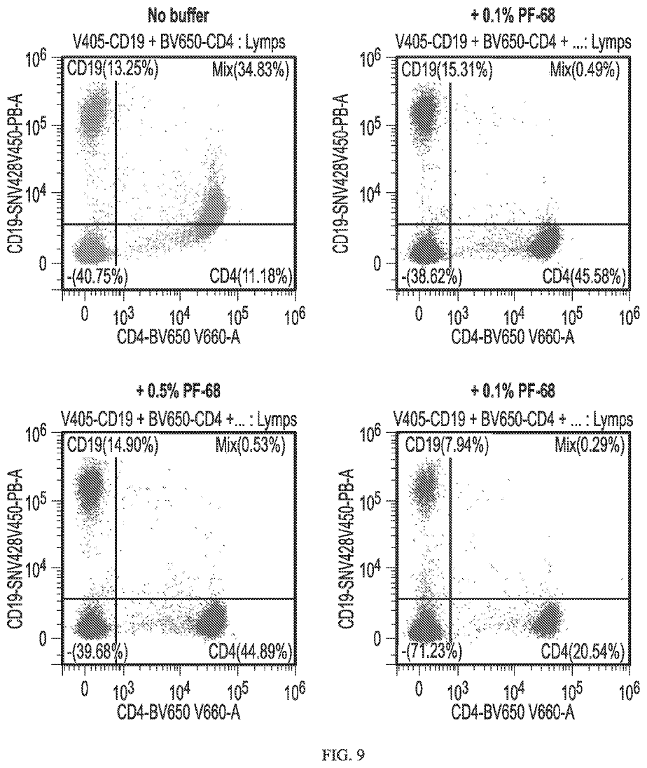

Nonionic surfactant was found to be a desirable additive for reducing non-specific polymer dye conjugate interactions in staining buffer compositions with UV-absorbing polymers or quenched UV polymers. The effect of different concentrations of nonionic surfactant alone on FCA of stained and lysed cells using a mixture of two different polymer dye conjugates was evaluated. FIG. 9 shows FCA dot plots of stained and lysed cells with a mixture of CD4-BV650 (BD Biosciences) and CD19-SNv428 (Beckman Coulter Life Sciences) without buffer (left panel), with 0.1% PF-68 (second from left panel), 0.5% PF-68 (second from right panel), and 1% PF-68 (wt/vol) (right panel). The presence of increasing concentration of PF-68 (0.1-1% w/vol) is associated with decreased non-specific interactions in the mixture as evidenced by improved separation compared to without PF-68.

### Example 11. Flow Cytometry Performance of Staining Buffer with Optional Zwitterionic Surfactant in Mixture of Two Different Polymer Dye Conjugates

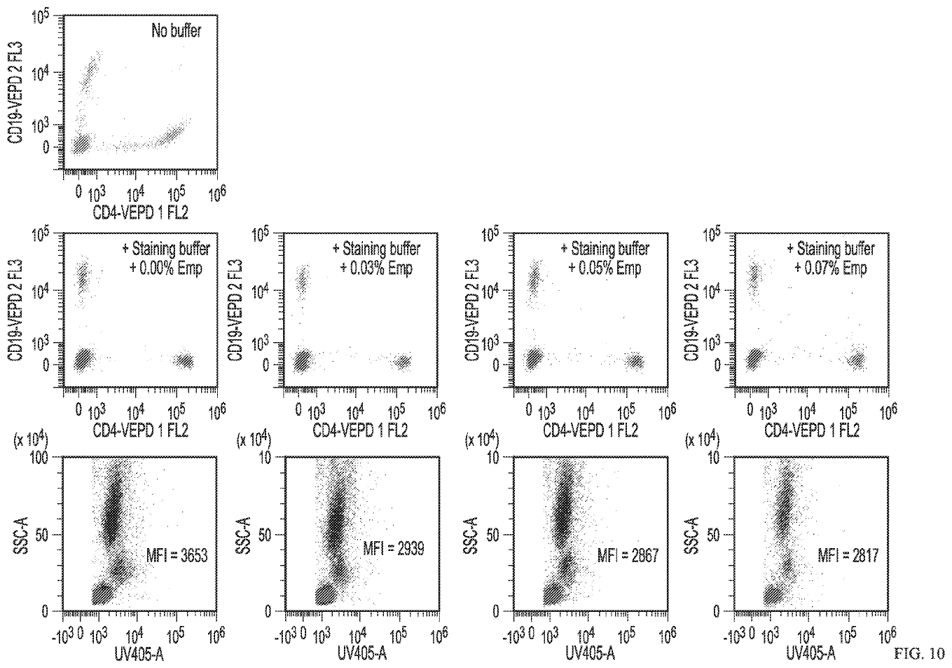

In some instances, it may desirable to also add a zwitterionic surfactant to the compositions according to the disclosure. FIG. 10 shows stained cells without buffer (Top panel), with combined staining buffer (composition of UV polymer+PF-68) and Empigen in various concentrations (0, 0.03%, 0.05%, and 0.07%) (middle panels: left, second from left, second from right, and right, respectively) were evaluated. The presence of various concentrations of Empigen in staining buffer do not affect the performance of staining buffer. Bottom panels: MFI values of monocytes indicate that with the addition of various concentrations of Empigen to staining buffer (bottom panels: from left to right) show decreased non-specific binding to cells as compared with control sample (bottom left panel).

### EXEMPLARY EMBODIMENTS

The following exemplary embodiments are provided, the numbering of which is not to be construed as designating levels of importance:

Embodiment 1a provides a UV-absorbing polymer having the structure of Formula I:

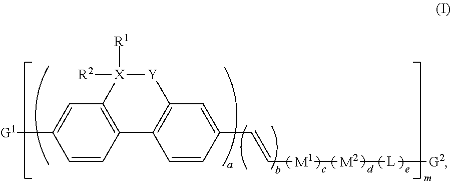

wherein
each X is independently selected from the group consisting of C and Si;
each Y is independently selected from the group consisting of a bond, CR¹R², CHR¹, CHR², SiHR², SiHR¹, and SiR¹R², and when Y is a bond X is directly bonded to both rings;
   each R¹ is independently selected from the group consisting of a water-solubilizing moiety, alkyl, alkene, alkyne, cycloalkyl, haloalkyl, (hetero)aryloxy, (hetero)arylamino, aryl, heteroaryl, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphoramide,

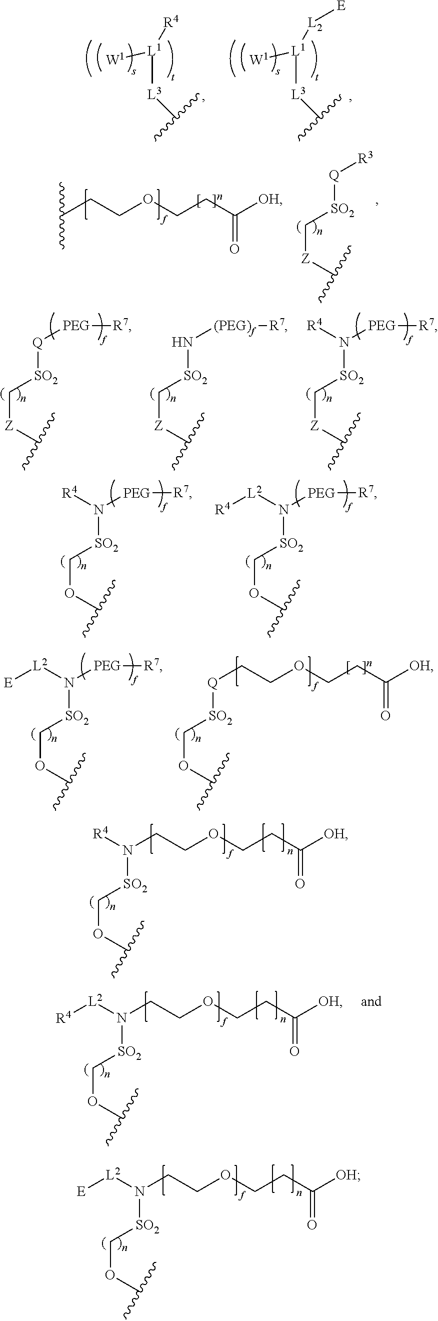

each R² is independently selected from the group consisting of a water-solubilizing moiety, a linker moiety, H, alkyl, alkene, alkyne, cycloalkyl, haloalkyl, alkoxy, (hetero)aryloxy, aryl, heteroaryl, (hetero)arylamino, a PEG group, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

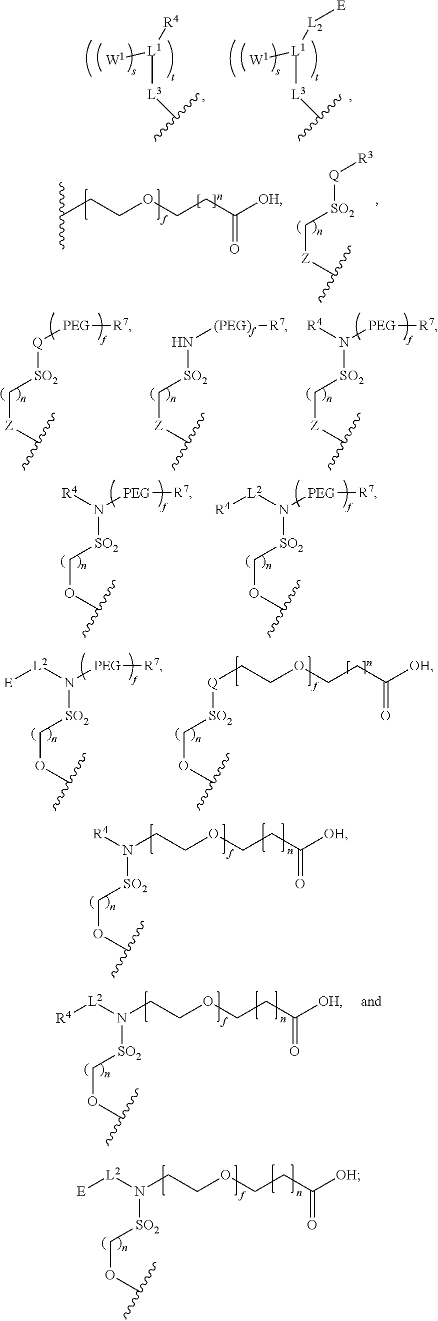

each R³ is independently selected from the group consisting of H, alkyl, alkene, alkyne, cycloalkyl, haloalkyl, alkoxy, (hetero)aryloxy, aryl, (hetero)arylamino, a w ater-solubilizing moiety, and a PEG group;
   each Z is independently selected from the group consisting of CH₂, CHR₄, O, NH, and NR⁴;
   each Q is independently selected from the group consisting of a bond, NH, NR⁴, C₁-C₁₂ alkylene, CHR⁴, and CH₂;
each R⁴ is independently selected the group consisting of H, a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, halogen, hydroxy, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OCH₃ wherein each x′ is independently an integer from 0-20 and each y′ is independently an integer from 0-50, Z—(CH₂)ₙ—SO₂-Q-R³, a C₂-C₁₈ (hetero)aryl group, amide, amine, carbamate, carboxylic acid, carboxylate ester, maleimide, activated ester, N-hydroxysuccinimidyl, hydrazine, hydrazone, azide, aldehyde, thiol, and protected groups thereof; each W¹ is independently a water-solubilizing moiety;
L¹, L², and L are each independently selected linker moieties;
each E is independently selected from the group consisting of a chromophore, a functional moiety, a substrate, and a binding partner;
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, and C₂-C₁₂ carboxylate ester;
at least one of R¹, R², R³, or R⁴ comprises a water-solubilizing moiety;
each M¹ is independently selected from the group consisting of an R⁴- and/or trifluoromethyl-substituted arylene that is optionally further substituted, an R⁴- and/or trifluoromethyl-substituted heteroarylene that is optionally further substituted, an R₄- and/or trifluoromethyl-substituted 9,10-dihydrophenanthrene that is optionally further substituted, and a binaphthyl that is optionally substituted;
each M² is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than M¹, and wherein M² and M¹ are evenly or randomly distributed along the polymer main chain;
each optional linker L is an aryl or heteroaryl group evenly or randomly distributed along the polymer main chain and that is substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule, or binding partner;
G¹ and G² are each independently selected from the group consisting of an modified polymer terminus and a modified polymer terminus, optionally conjugated to E;
a, c, d, and e define the mol % of each unit within the structure which each can be evenly or randomly repeated along the polymer main chain and where a is a mol % from 10 to 100%, c is a mol % from >0 to 90%, each d is a mol % from 0 to 90%, and each e is a mol % from 0 to 25%;
each b is independently 0 or 1;
each f is independently an integer from 0 to 50;
m is an integer from 1 to about 10,000;
each n is independently an integer from 1 to 20;
s is 1 or 2; and
t is 0, 1, 2, or 3.

Embodiment 1b provides the polymer of Embodiment 1a, wherein the polymer has the structure of Formula I:

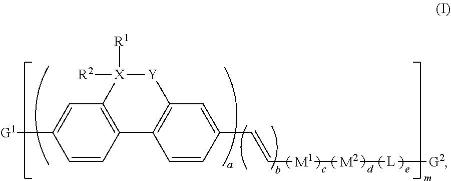

wherein
each X is independently selected from C and Si;
each Y is independently selected from a bond, CR¹R², and SiR¹R², and
when Y is a bond X is directly bonded to both rings; each R¹ is independently selected from polyethylene glycol (PEG), ammonium alkyl salt, ammonium alkyloxy salt, ambenonium oligoether salt, sulfonate alkyl salt, sulfonate alkoxy salt, sulfonamido oligoether, and —Z—(CH₂)ₙ—SO₂-Q-R³;
each R² is independently selected from H, alkyl, alkene, alkyne, cycloalkyl, haloalkyl, alkoxy, (hetero)aryloxy, aryl, (hetero)arylamino, a PEG group, ammonium alkyl salt, ammonium alkyloxy salt, ammonium oligoether salt, sulfonate alkyl salt, sulfonate alkoxy salt, sulfonate oligoether salt, sulfonamido oligoether, and —Z—(CH₂)ₙ—SO₂-Q-R³;
each R³ is independently selected from H, alkyl, alkene, alkyne, cycloalkyl, haloalkyl, alkoxy, (hetero)aryloxy, aryl, (hetero)arylamino, and a PEG group;
each Z is independently selected from CH₂, CHR⁴, O, NIT, and NR⁴; each Q is independently selected from a bond, NH, NR⁴, C₁-C₁₂ alkylene, CHR⁴, and CH₂;
each R⁴ is independently selected from a chromophore, a linked chromophore, halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₈ (hetero)aryloxy, C₂-C₁₈ (hetero)arylamino, (CH₂)_{x′}(OCH₂—CH₂)_{y′}OCH₃ wherein each x′ is independently an integer from 0-20 and each y′ is independently an integer from 0-50, —Z—(CH₂)ₙ—SO₂-Q-R³, and a C₂-C₁₈ (hetero)aryl group;
each M¹ is independently selected from an R⁴- and/or trifluoromethyl-substituted arylene that is optionally further substituted, an R^{L}- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted;
each M² is independently selected from an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than M¹;
each linker L is an aryl or heteroaryl group evenly or randomly distributed along the polymer main chain and that is substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule or binding partner;
G¹ and G² are each independently selected from hydrogen, halogen, alkyne, halogen substituted aryl, silyl, diazonium salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), or optionally substituted fluorene, wherein the optionally substituted aryl, heteroaryl, fluorene, or DHP may be substituted with one or more pendant chains terminated with a functional group, for example, selected from amine, carbamate, carboxylic acid, carboxylate, maleimide, activated ester, N-hydroxylsuccinimidyl, hydrazine, hydrazide, hydrazone, azide, alkyne, aldehyde, thiol, and protected groups thereof, for conjugation to a substrate or binding partner or conjugated to a substrate or binding partner;
a, c, d, and e define the mol % of each unit within the structure which each can be evenly or randomly repeated along the polymer main chain and where a is a mol % from 10 to 100%, e is a mol % from >0 to 90%, each d is a mol % from 0 to 90%, and each e is a mol % from 0 to 25%;
each b is independently 0 or 1;
m is an integer from 1 to about 10,000; and
each n is independently an integer from 1 to 20.

Embodiment 2a provides the polymer any one of Embodiments 1a and 1b, wherein the polymer has the structure of Formula II:

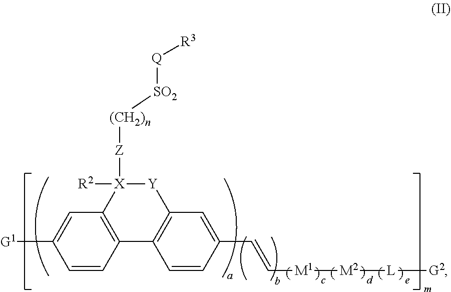

Embodiment 3 provides the polymer of any one of Embodiments 1a-2b, wherein the polymer has the structure of Formula III:

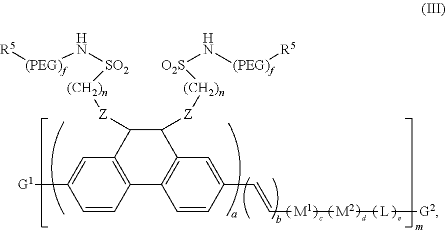

wherein each f is independently an integer from 0 to 50 and each R⁵ is independently selected from the group consisting of H, C₁-C₁₂ alkyl, C₂-C₁₂ alkene, C₂—C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, and C₁-C₁₂ alkoxy.

Embodiment 4 provides the polymer of any one of Embodiments 1a-3, wherein the polymer has the structure of Formula IV:

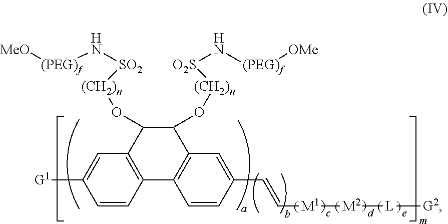

wherein each f is independently an integer from 0 to 50.

Embodiment 5 provides the polymer of any one of Embodiments 1a-4, wherein the polymer has the structure of Formula V:

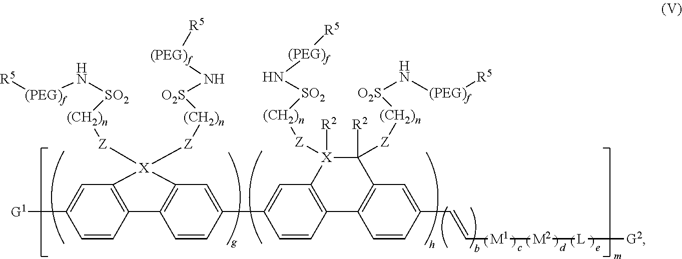

wherein g and h together is a mol % from 10 to 100%, each f is independently an integer from 0 to 50, and each R⁵ is independently selected from the group consisting of H, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, and C₁-C₁₂ alkoxy.

Embodiment 6 provides the polymer of any one of Embodiments 1a-5, wherein the polymer has the structure of Formula VI:

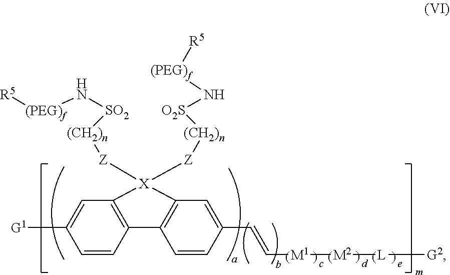

wherein each f is independently an integer from 0 to 50, and each R⁵ is independently selected from the group consisting of H, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, and C₁-C₁₂ alkoxy.

Embodiment 7 provides the polymer of any one of Embodiments 1a-6, wherein the polymer has the structure of Formula VII:

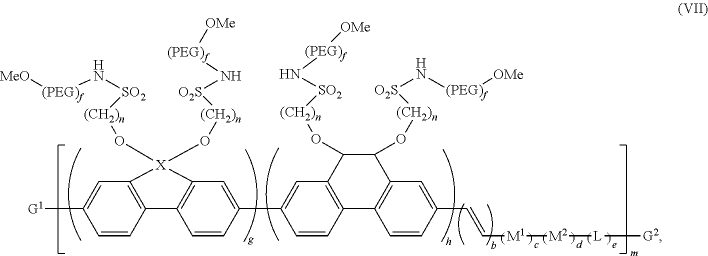

wherein g and h together is a mol from 10 to 100% and each f is independently an integer from 0 to 50.

Embodiment 8 provides the polymer of any one of Embodiments 1a-7, wherein the polymer has the structure of Formula VIII:

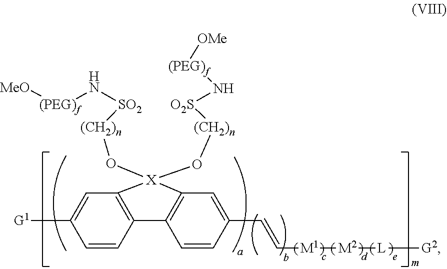

wherein each f is independently an integer from 0 to 50.

Embodiment 9a provides the polymer of any one of Embodiments 1a-8, wherein the polymer comprises a structure according to Formula XIV:

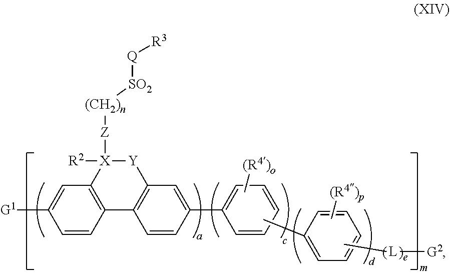

wherein each of R², R³, G¹, G², L, Q, X, Y, Z, a, b, c, e, n, and m is independently as described herein; each R^{4′} is independently selected from F, Cl, —CH₃, —CF₃, and —(OCH₂CH₂)_{f}OR⁹; each R^{4″} is independently selected from F, Cl, —CH₃, —CF₃, and —(OCH₂CH₂)_{f}OR⁹; R⁹ is C₁-C₈ alkyl; each f is independently an integer from 0 to 50, or 10-20; each o is independently an integer selected from 1, 2, 3, or 4; and each p is independently an integer selected from 1, 2, 3, or 4.

Embodiment 9b provides the polymer of any one of Embodiments 1a-9a, wherein each M¹ is independently a fluorine-substituted arylene having 1-4 fluorine substituents, or wherein each M¹ is independently a halide-, MeO-PEG-CH₂—, and/or MeO-PEG-substituted arylene (e.g., phenylene) that is optionally further substituted.

Embodiment 10 provides the polymer of any one of Embodiments 1a-9b, wherein each M¹ is independently a fluorine-substituted phenylene having 1-4 fluorine substituents, wherein the phenylene is optionally further substituted.

Embodiment 11 provides the polymer of any one of Embodiments 1a-10, wherein each M¹ is independently a fluorine-substituted phenylene having 2 or 3 fluorine substituents.

Embodiment 12 provides the polymer of any one of Embodiments 1a-11, wherein each M¹ is a difluoro-substituted phenylene.

Embodiment 13 provides the polymer of any one of Embodiments 1a-12, wherein each M¹ is independently selected from:
a phenylene having the 1- and 4-positions thereof substituted into the backbone of the polymer and that is difluoro-substituted with fluorine at the 2- and 3-positions, the 2- and 5-positions, or at the 2- and 6-positions,
a phenylene having the 1- and 4-positions thereof substituted into the backbone of the polymer and that is trifluoro-substituted with fluorine at the 2-, 3-, and 5-positions,
a phenylene having the 1- and 3-positions thereof substituted into the backbone of the polymer and that is difluoro-substituted with fluorine at the 2- and 4-positions, the 2- and 5-positions, the 4- and 5-positions, or the 4- and 6-positions, and
a phenylene having the 1- and 3-positions thereof substituted into the backbone of the polymer and that is trifluoro-substituted with fluorine at the 4-, 5-, and 6-positions, at the 2-, 4-, and 5-positions, or at the 2-, 4-, and 6-positions.

Embodiment 14 provides the polymer of any one of Embodiments 1a-13, wherein each M¹ is independently selected from:
a phenylene having the 1- and 4-positions thereof substituted into the backbone of the polymer and that is difluoro-substituted with fluorine at the 2- and 3-positions, the 2- and 5-positions, or at the 2- and 6-positions, and
a phenylene having the 1- and 3-positions thereof substituted into the backbone of the polymer and that is difluoro-substituted with fluorine at the 2- and 4-positions, the 2- and 5-positions, the 4- and 5-positions, or the 4- and 6-positions.

Embodiment 15 provides the polymer of any one of Embodiments 1a-14, wherein each M¹ is independently selected from:

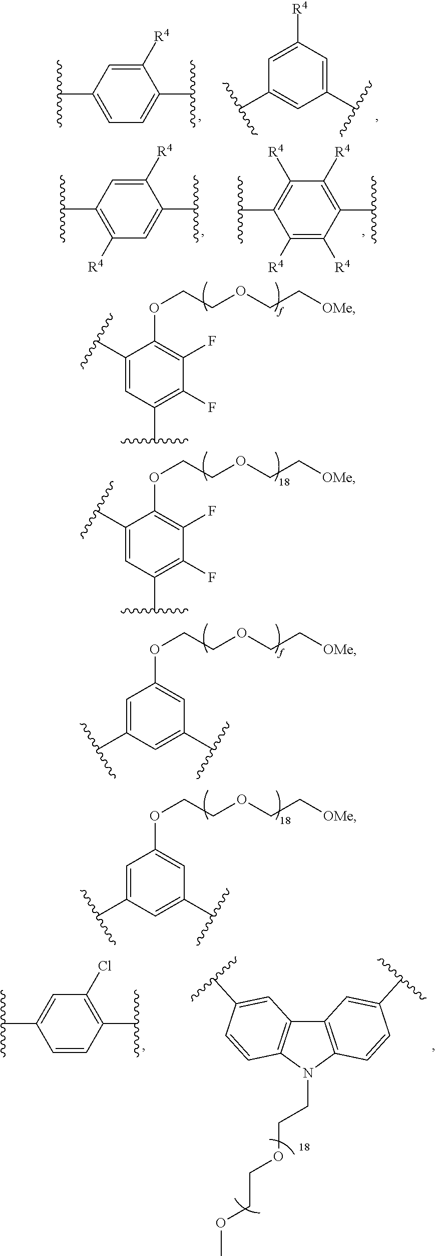

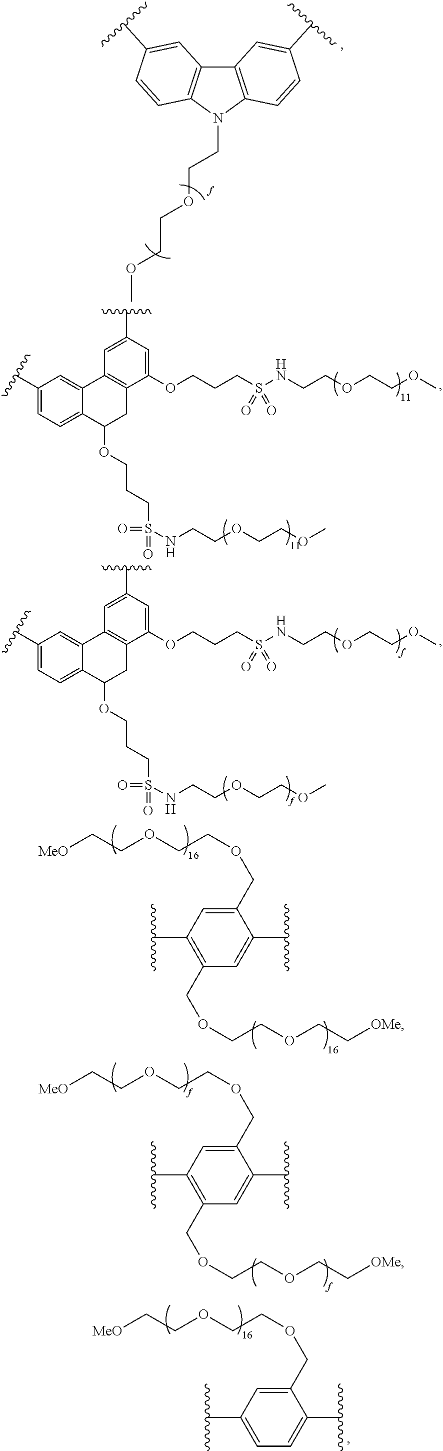

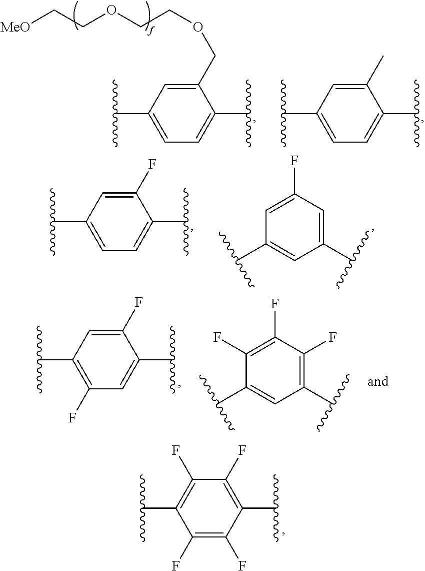

wherein each f is independently an integer from 0 to 50, 10 to 20, or 11 to 18.

Embodiment 16 provides the polymer of any one of Embodiments 1a-15, wherein each M¹ is:

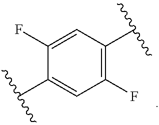 [SMILES: *c1cc(F)c(*)cc1F]

Embodiment 17 provides the polymer of any one of Embodiments 1a-16, wherein each M¹ is a phenylene having the 1- and 4-positions thereof substituted into the backbone of the polymer and that is 2,5-difluoro substituted.

Embodiment 18 provides the polymer of any one of Embodiments 1a-17, wherein each M² is independently a fluorine-substituted arylene having 1-4 fluorine substituents, or wherein each M² is independently a halide-, MeO-PEG-CH₂—, and/or MeO-PEG-substituted arylene (e.g., phenylene) that is optionally further substituted.

Embodiment 19 provides the polymer of any one of Embodiments 1a-18, wherein each M² is independently a fluorine-substituted phenylene having 1-4 fluorine substituents, wherein the phenylene is optionally further substituted.

Embodiment 20 provides the polymer of any one of Embodiments 1a-19, wherein each M² is independently a fluorine-substituted phenylene having 2 or 3 fluorine substituents.

Embodiment 21 provides the polymer of any one of Embodiments 1a-20, wherein each M² is a trifluoro-substituted phenylene.

Embodiment 22 provides the polymer of any one of Embodiments 1a-21, wherein each M² is independently selected from:
a phenylene having the 1- and 4-positions thereof substituted into the backbone of the polymer and that is trifluoro-substituted with fluorine at the 2-, 3-, and 5-positions, and
a phenylene having the 1- and 3-positions thereof substituted into the backbone of the polymer and that is trifluoro-substituted with fluorine at the 4-, 5-, and 6-positions, at the 2-, 4-, and 5-positions, or at the 2-, 4-, and 6-positions.

Embodiment 23 provides the polymer of any one of Embodiments 1a-22, wherein each M² is

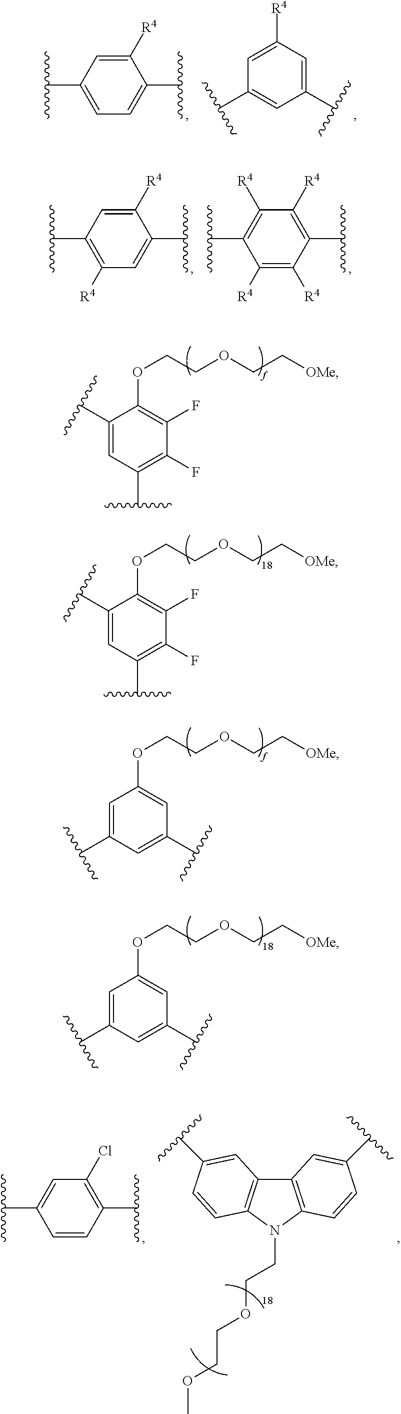

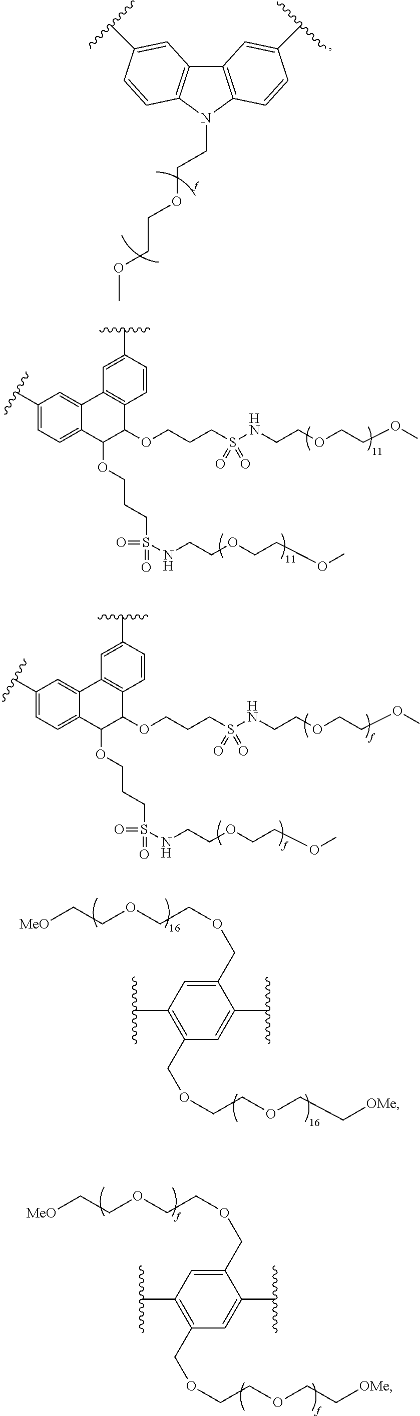

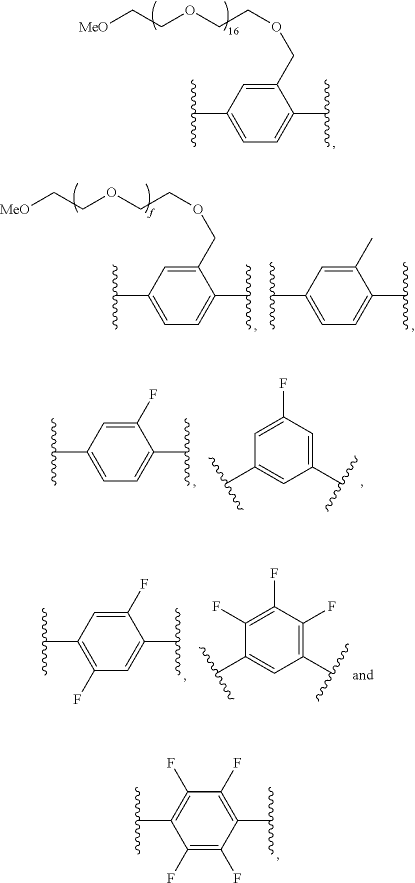

wherein each f is independently an integer from 0 to 50, 10 to 20, or 11 to 18, and wherein M² is different than M¹.

Embodiment 24 provides the polymer of any one of Embodiments 1a-23, wherein each M² is a phenylene having the 1- and 3-positions thereof substituted into the backbone of the polymer and that is 4,5,6-trifluoro substituted.

Embodiment 25 provides the polymer of any one of Embodiments 1a-24, wherein each L is independently selected from:

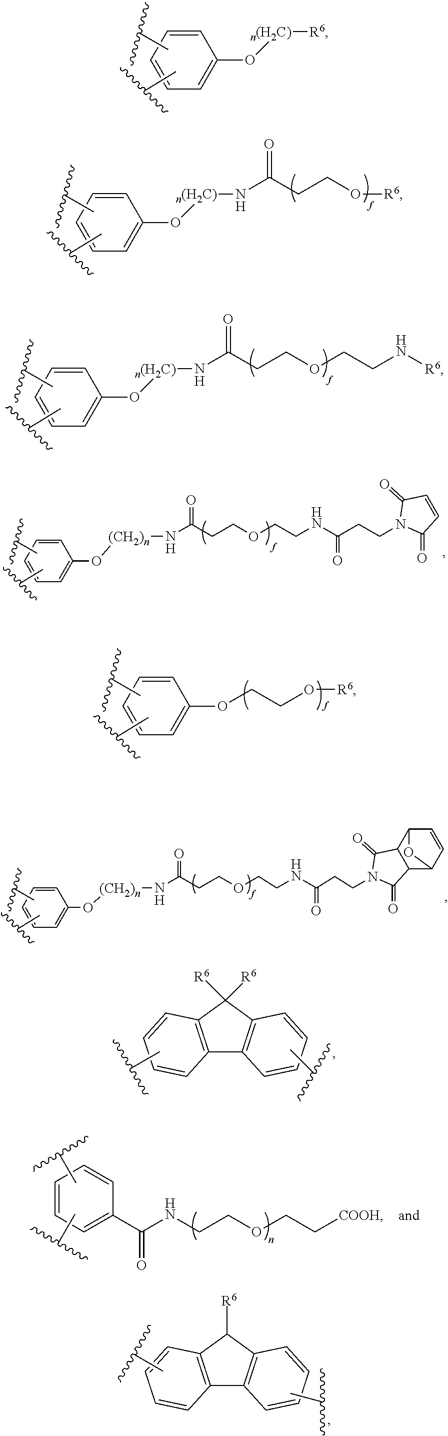

wherein
each R⁶ is independently selected from H, OH, SH, NHCOO-t-butyl, (CH₂)ₙCOOH, (CH₂)ₙCOOCH₃, (CH₂)ₙNH₂, (CH₂)ₙNH—(CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₂)Cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCF₁₂CH₂)_{f}OCH₃, N-maleimide, halogen, C₂-C₁₂ alkene, C₂-C₁₂ alkyne, C₃-C₁₂ cycloalkyl, C₁-C₁₂ haloalkyl, C₁-C₁₂ (hetero)aryl, C₁-C₁₂ (hetero)arylamino, and benzyl optionally substituted with one or more halogen, hydroxyl, C₁-C₁₂ alkoxy, or (OCH₂CH₂)_{f}OCH₃;
each f is independently an integer from 0 to 50; and
each n is independently an integer from 1 to 20.

Embodiment 26 provides the polymer of any one of Embodiments 1a-25, wherein G¹ and G² are each independently selected from optionally substituted dihydrophenanthrene (DHP), optionally substituted fluorene, aryl substituted with one or more pendant chains terminated with a functional group, and a heteroaryl substituted with one or more pendant chains terminated with a functional group.

Embodiment 27 provides the polymer of any one of Embodiments 1a-26, wherein G¹ and G² are each independently selected from:

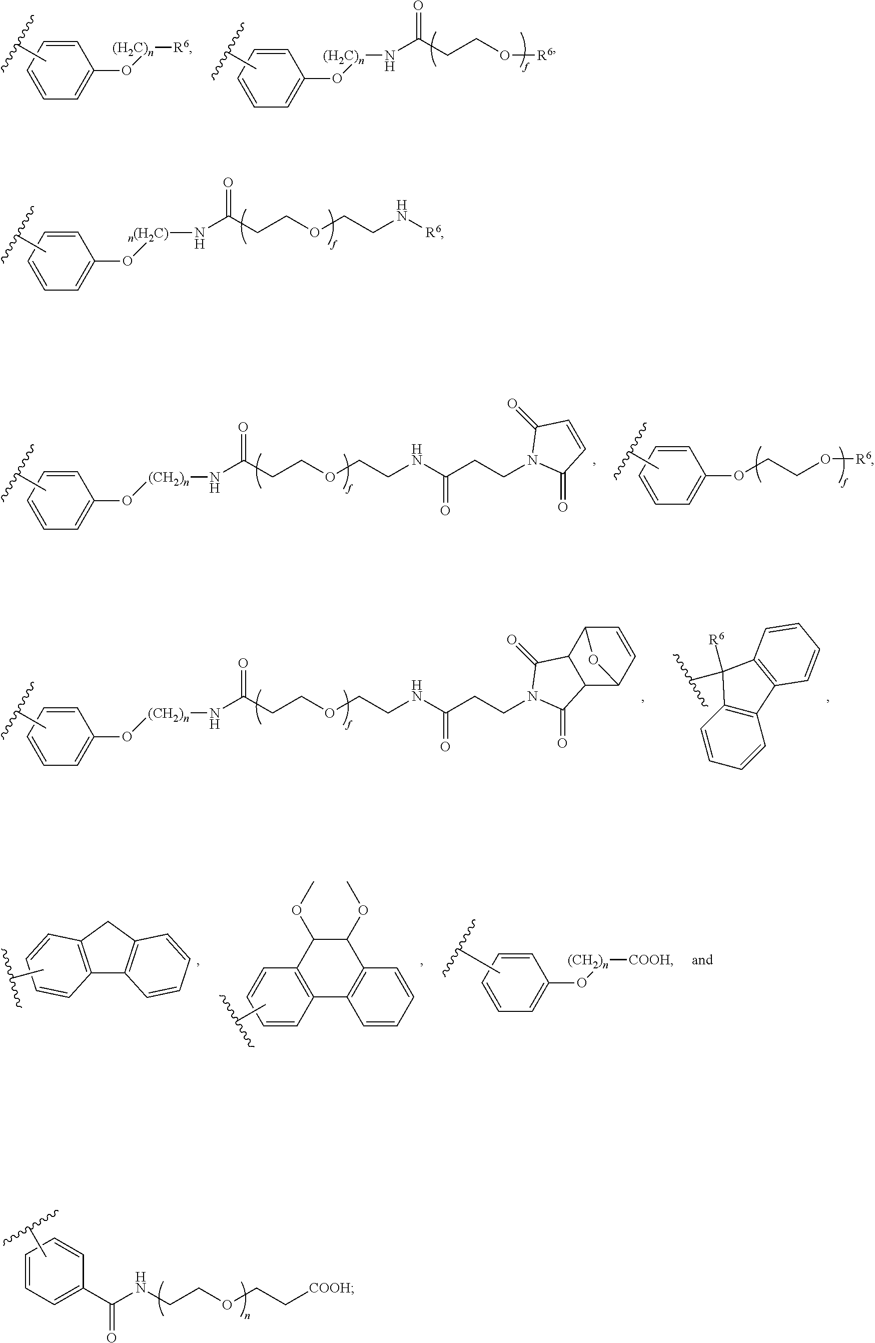

wherein
each R¹ is independently selected from H, OH, SH, NHCOO-t-butyl, (CH₂)ₙCOOH, (CH₂)ₙCOOCH₃, (CH₂)ₙNH₂, (CH₂)ₙNH—(CH₂)ₙ—CH₃, (CH₂)ₙNHCOOH, (CH₂)ₙNHCO—(CH₂), —CO—(CH₂)ₙ—CH₃, (CH₂)% NHCOO—(CH₂)ₙ—CH₃, (CH₂)ₙNHCOOC(CH₃)₃, (CH₂)ₙNHCO(C₃-C₁₂)cycloalkyl, (C₁-C₁₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃₋₁₂ cycloalkyl, C₁-C₁₂ haloalkyl, C₁-C₁₂ (hetero)aryl, C₁-C₁₂ (hetero)arylamino, and benzyl optionally substituted with one or more halogen, hydroxyl, C₁-C₁₂ alkoxy, or (OCH₂CH₂)_{f}OCH₃;
each f is independently an integer from 0 to 50; and
each n is independently an integer from 1 to 20.

Embodiment 28 provides the polymer of any one of Embodiments 1a-27, wherein the polymer has the structure of Formula IX:

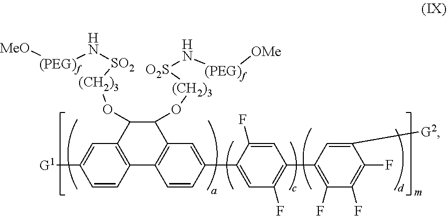

wherein f is independently an integer from 0 to 50.

Embodiment 29 provides the polymer of any one of Embodiments 1a-28, wherein a molar ratio of M¹ to M² groups is 0.5:1 to 1.5:1.

Embodiment 30 provides the polymer of any one of Embodiments 1a-29, wherein a molar ratio of M¹ to M² groups is 0.7:1 to 1.3:1.

Embodiment 31 provides the polymer of any one of Embodiments 1a-30, wherein a molar ratio of M¹ to M² groups is 0.9:1 to 1.1:1.

Embodiment 32 provides the polymer of any one of Embodiments 1a-31, wherein a molar ratio of M¹ to M² groups is about 1:1.

Embodiment 33 provides the polymer of any one of Embodiments 1a-32, wherein b is 0.

Embodiment 34 provides the polymer of any one of Embodiments 1a-33, wherein a is 25% to 75%.

Embodiment 35 provides the polymer of any one of Embodiments 1a-34, wherein a is 35% to 65%.

Embodiment 36 provides the polymer of any one of Embodiments 1a-35, wherein a is 45% to 55%.

Embodiment 37 provides the polymer of any one of Embodiments 1a-36, wherein e is 5% to 80%.

Embodiment 38 provides the polymer of any one of Embodiments 1a-37, wherein c is 10% to 40%.

Embodiment 39 provides the polymer of any one of Embodiments 1a-38, wherein c is 15% to 35%.

Embodiment 40 provides the polymer of any one of Embodiments 1a-39, wherein e is 20% to 30%.

Embodiment 41 provides the polymer of any one of Embodiments 1a-40, wherein d is 0%.

Embodiment 42 provides the polymer of any one of Embodiments 1a-41, wherein d is 5% to 80%.

Embodiment 43 provides the polymer of any one of Embodiments 1a-42, wherein d is 10% to 40%.

Embodiment 44 provides the polymer of any one of Embodiments 1a-43, wherein d is 15% to 35%.

Embodiment 45 provides the polymer of any one of Embodiments 1a-44, wherein d is 20 to 30%.

Embodiment 46 provides the polymer of any one of Embodiments 1a-45, wherein e is 0%.

Embodiment 47 provides the polymer of any one of Embodiments 1a-46, wherein e is 0% to 20%.

Embodiment 48a provides the polymer of any one of Embodiments 1a-47, wherein at least one R² is —Z—(CH₂)ₙ—SO₂—N(chromophore)-R³.

Embodiment 49 provides the polymer of any one of Embodiments 1a-48, wherein the polymer has an absorption maximum at 320 nm to 380 nm.

Embodiment 50 provides the polymer of any one of Embodiments 1a-49, wherein the polymer has an absorption maximum at 340 nm to 360 nm.

Embodiment 51 provides the polymer of any one of Embodiments 1a-50, wherein the polymer has an absorption maximum at 345 nm to 356 nm.

Embodiment 52 provides the polymer of any one of Embodiments 1a-51, wherein the polymer has an emission maximum of 380 nm to 430 nm.

Embodiment 53 provides the polymer of any one of Embodiments 1a-52, wherein the polymer has an emission maximum of 406 nm to 415 nm.

Embodiment 54 provides the polymer of any one of Embodiments 1a-53, further comprising a binding partner linked to the polymer.

Embodiment 55 provides the polymer of Embodiment 54, wherein the binding partner is an antibody.

Embodiment 56a provides a method for detecting an analyte in a sample comprising:
contacting a sample that is suspected of containing the analyte with a binding partner conjugated to a polymer comprising the structure of Formula I:

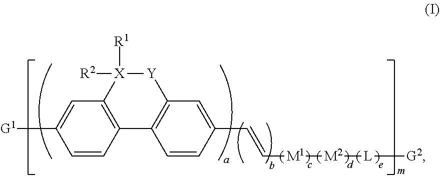

wherein
each X is independently selected from the group consisting of C and Si;
each Y is independently selected from the group consisting of a bond, CR¹R², CHR¹, CHR², and SiR¹R², and when Y is a bond X is directly bonded to both rings;
each R¹ is independently selected from the group consisting of a water-solubilizing moiety, a linker moiety, alkyl, alkene, alkyne, cycloalkyl, haloalkyl, (hetero)aryloxy, (hetero)arylamino, aryl, heteroaryl, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide,

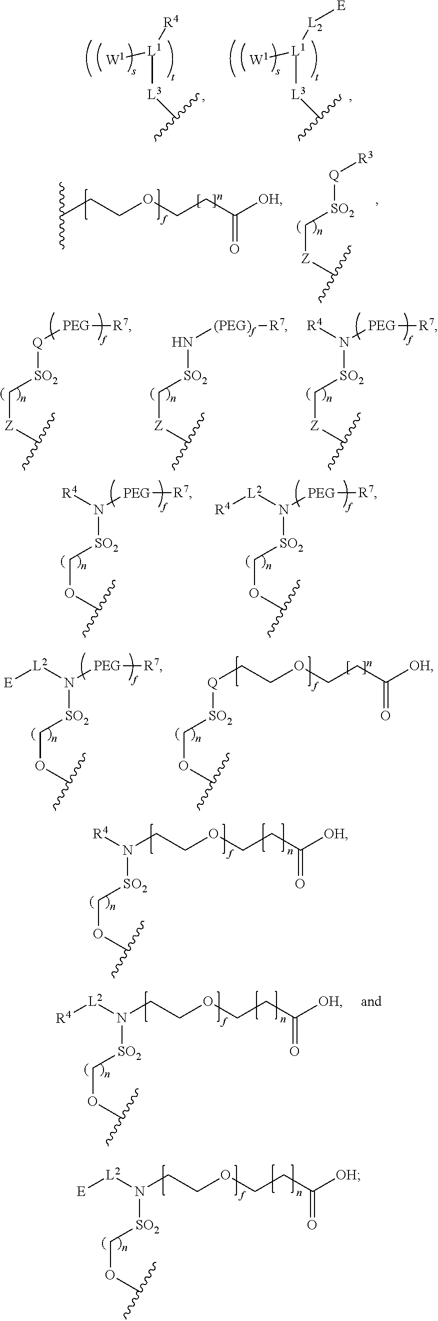

each R₂ is independently selected from the group consisting of a water-solubilizing moiety, a linker moiety, H, alkyl, alkene, alkyne, cycloalkyl, haloalkyl, alkoxy, (hetero)aryloxy, aryl, heteroaryl, (hetero)arylamino, a PEG group, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide,

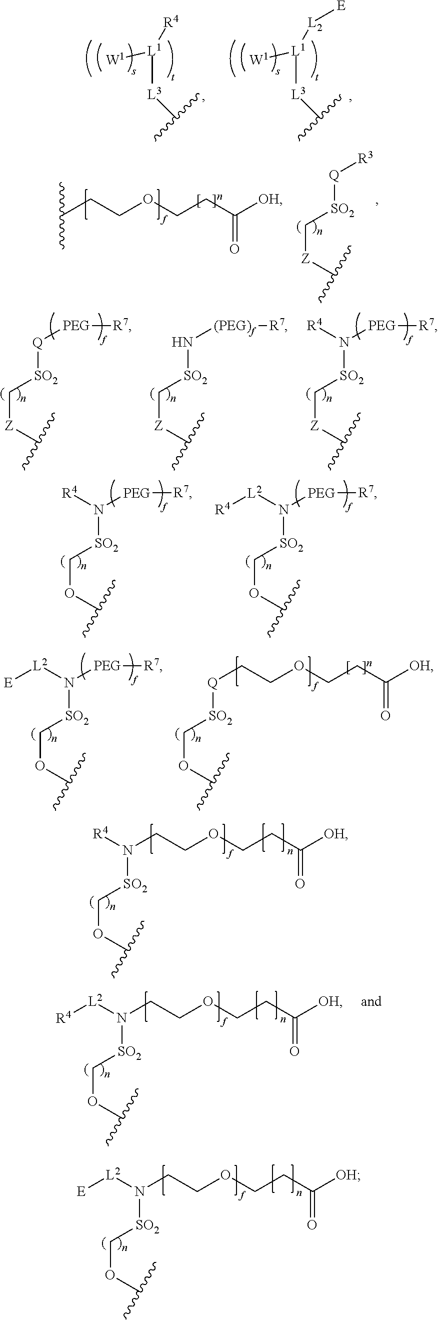

each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, and a PEG group;
each Z is independently selected from the group consisting of CH₂, CHR⁴, O, NH, and NR⁴;
each Q is independently selected from the group consisting of a bond, NH, NR⁴, C₁-C₁₂ alkylene, CHR⁴, and CH₂;
each R⁴ is independently selected the group consisting of H, a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OCH₃ wherein each x′ is independently an integer from 0-20 and each y′ is independently an integer from 0-50, Z—(CH₂)ₙ—SO₂-Q-R³, a C₂-C₁₈ (hetero)aryl group, amide, amine, carbamate, carboxylic acid, carboxylate ester, maleimide, activated ester, N-hydroxysuccinimidyl, hydrazine, hydrazone, azide, aldehyde, thiol, and protected groups thereof;
each W¹ is independently a water-solubilizing moiety;
L¹, L², and L³ are each independently selected linker moieties;
each E is independently selected from the group consisting of a chromophore, a functional moiety, a substrate, and a binding partner;
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂haloalkyl, C₁-C₁₂ alkoxy, C₂-C₁₈ (hetero)aryloxy, C₂-C₁₈ (hetero)arylamino, C₂-C₁₂ carboxylic acid, and C₂-C₁₂ carboxylate ester;
at least one of R¹, R², R³, or R⁴ comprises a water-solubilizing moiety;
each M¹ is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted;
each M² is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than M¹, and wherein M² and M¹ are evenly or randomly distributed along the polymer main chain;
each optional linker L is an aryl or heteroaryl group evenly or randomly distributed along the polymer main chain and that is substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule, or binding partner;
G¹ and G² are each independently selected from the group consisting of an unmodified polymer terminus and a modified polymer terminus, optionally conjugated to E;
a, c, d, and e define the mol % of each unit within the structure which each can be evenly or randomly repeated along the polymer main chain and where a is a mol % from 10 to 100%, c is a mol % from >0 to 90%, each d is a mol % from 0 to 90%, and each e is a mol % from 0 to 25%;
   each b is independently 0 or 1;
   each f is independently an integer from 0 to 50;
m is an integer from 1 to about 10,000;
each n is independently an integer from 1 to 20;
s is 1 or 2; and
t is 0, 1, 2, or 3.

Embodiment 56b provides the method of Embodiment 56a, wherein the polymer having the structure of Formula I comprises wherein
each X is independently selected from C and Si;
each Y is independently selected from a bond, CR¹R², and SiR¹R², and when Y is a bond X is directly bonded to both rings;
each R¹ is independently selected from polyethylene glycol (PEG), ammonium alkyl salt, ammonium alkyloxy salt, ammonium oligoether salt, sulfonate alkyl salt, sulfonate alkoxy salt, sulfonamido oligoether, and —Z—(CH₂)ₙ—SO₂-Q-R³;
each R² is independently selected from H, alkyl, alkene, alkyne, cycloalkyl, haloalkyl, alkoxy, (hetero)aryloxy, aryl, (hetero)arylamino, a PEG group, ammonium alkyl salt, ammonium alkyloxy salt, ammonium oligoether salt, sulfonate alkyl salt, sulfonate alkoxy salt, sulfonate oligoether salt, sulfonamido oligoether, and —Z—(CH₂)ₙ—SO₂-Q-R³;
each R³ is independently selected from H, alkyl, alkene, alkyne, cycloalkyl, haloalkyl, alkoxy, (hetero)aryloxy, aryl, (hetero)arylamino, and a PEG group;
each Z is independently selected from selected from CH₂, CHR⁴, O, NH, and NR⁴;
each Q is independently selected from a bond, NH, NR⁴, C₁-C₁₂ alkylene, and CH₂;
each R⁴ is independently selected from a chromophore, halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OCH₃ wherein each x′ is independently an integer from 0-20 and each y′ is independently an integer from 0-50, —Z—(CH₂)ₙ—SO₂-Q-R³, and a C₂-C₁S (hetero)aryl group;
each M¹ is independently selected from an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted;
each M² is independently selected from an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than M¹;
each linker L is an aryl or heteroaryl group evenly or randomly distributed along the polymer main chain and that is substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof for conjugation to another substrate, acceptor dye, molecule or binding partner;
G¹ and G² are each independently selected from the group consisting of hydrogen, halogen, alkyne, halogen substituted aryl, silyl, diazonium, salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), and optionally substituted fluorene, wherein the substituted aryl, heteroaryl, fluorene, or DHP are substituted with one or more pendant chains terminated with a functional group, for example, selected from amine, carbamate, carboxylic acid, carboxylate, maleimide, activated ester, N-hydroxylsuccinimidyl, hydrazine, hydrazide, hydrazone, azide, alkyne, aldehyde, thiol, and protected groups thereof, for conjugation to a substrate or binding partner;
a, c, d, and e define the mol % of each unit within the structure which each can be evenly or randomly repeated along the polymer main chain and where a is a mol % from 10 to 100%, c is a mol % from >0 to 90%, each d is a mol % from 0 to 90%, and each e is a mol % from 0 to 25%;
each b is independently 0 or 1;
m is an integer from 1 to about 10,000;
each n is independently an integer from 1 to 20; and
the binding agent is capable of interacting with the analyte or a target-associated biomolecule.

Embodiment 57 provides the method of Embodiment 56a or 56b, wherein the binding partner is a protein, peptide, affinity ligand, antibody, antibody fragment, sugar, lipid, nucleic acid, or an aptamer.

Embodiment 58 provides the method of any one of Embodiments 56a-57, wherein the binding partner is an antibody.

Embodiment 59 provides the method of any one of Embodiments 56a-58, wherein the method is configured for flow cytometry.

Embodiment 60 provides the method of any one of Embodiments 56a-59, wherein the binding partner is bound to a substrate.

Embodiment 61 provides the method of any one of Embodiments 56a-60, wherein the analyte is a protein expressed on a cell surface.

Embodiment 62 provides the method of any one of Embodiments 56a-61, wherein the method is configured as an immunoassay.

Embodiment 63 provides the method of any one of Embodiments 56a-62, wherein the method further comprises providing additional binding partners for detecting additional analytes simultaneously.

Embodiment 64 provides the polymer of any one of Embodiments 1a-55 or the method of any one of Embodiments 56a-63, wherein Y is a bond and R¹ and R² are each independently —Z—(CH₂)ₙ—SO₂-Q-R³.

Embodiment 65 provides the polymer of any one of Embodiments 1a-55 or the method of any one of Embodiments 56a-63, wherein each f is independently an integer from 5 to 30; and each n is independently an integer from 2 to 10.

Embodiment 66 provides the polymer of any one of Embodiments 1a-55 or the method of any one of Embodiments 56a-63, wherein each f is independently an integer from 10 to 25; and each n is independently an integer from 3 to 5.

Embodiment 67 provides the polymer of any one of Embodiments 1a-55, 64 or 65, wherein the acceptor dye is a quenching moiety.

Embodiment 68 provides the polymer of any one of Embodiments 1a-55, or 64-67, wherein the polymer does not comprise a binding partner.

Embodiment 69 provides the polymer or method of any one or any combination of Embodiments 1a-68 optionally configured such that all elements or options recited are available to use or select from.

Embodiment 70 provides a composition for use with at least one fluorescent polymer dye conjugated to a binding partner for use in staining a biological sample, the composition comprising: at least one UV-absorbing polymer dye or quenched UV polymer dye; optionally wherein the UV-absorbing polymer dye or quenched polymer dye comprises a structure according to any of Formulae I, I, III, IV, V, VI, VII, VIII, IX, X, XI, X, and/or XIV, or any one of Embodiments 1a-55; a nonionic surfactant; and a biological buffer, wherein the composition reduces non-specific binding of the at least one fluorescent polymer dye conjugate, when compared to the at least one fluorescent polymer dye conjugate in the absence of the composition.

Embodiment 71 provides the composition of Embodiment 70, wherein the quenched UV polymer dye comprises the UV-absorbing polymer dye comprising at least one quenching moiety, optionally 1-30, 2-20, or 2.5-10 quenching moieties.

Embodiment 72 provides the composition of Embodiment 70 or 71, wherein the quenching moieties are selected from the group consisting of DACYL, Dabcyl plus, 490Q, 425Q, and 505Q.

Embodiment 73 provides the composition of any one of Embodiments 70 to 72, wherein the nonionic surfactant is a poly(ethylene oxide)-poly(propylene oxide)-poly(ethylene oxide) triblock copolymer.

Embodiment 74 provides the composition of any one of Embodiments 70 to 73, wherein the nonionic surfactant comprises a structure according to formula (XII)

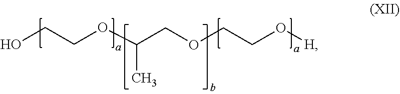

wherein each a is independently in the range of 2-130 and b is in the range of 15-67.

Embodiment 75 provides the composition of any one of Embodiments 70 to 74, wherein the composition further comprises an additional additive selected from the group consisting of a protein stabilizer, a preservative, and an additional surfactant, optionally wherein the additional surfactant is a zwitterionic surfactant or an ionic surfactant.

Embodiment 76 provides the composition of any one of Embodiments 70 to 75, wherein the composition comprises a plurality of fluorescent polymer dye conjugates, and the composition substantially reduces the non-specific binding between the plurality of fluorescent polymer dye conjugates.

1. A UV-absorbing polymer dye comprising the structure of Formula I: 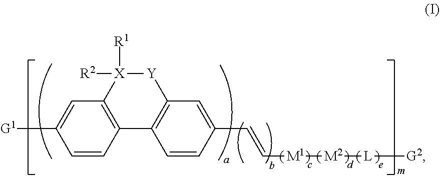
wherein
each X is independently selected from the group consisting of C and Si;
each Y is independently selected from the group consisting of a CR¹R², CHR¹, CHR², SiHR², SiHR¹, and SiR¹R²;
each R¹ is independently selected from the group consisting of a water-solubilizing moiety, a linker moiety, alkyl, alkene, alkyne, cycloalkyl, haloalkyl, (hetero)aryloxy, (hetero)arylamino, aryl, heteroaryl, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide, 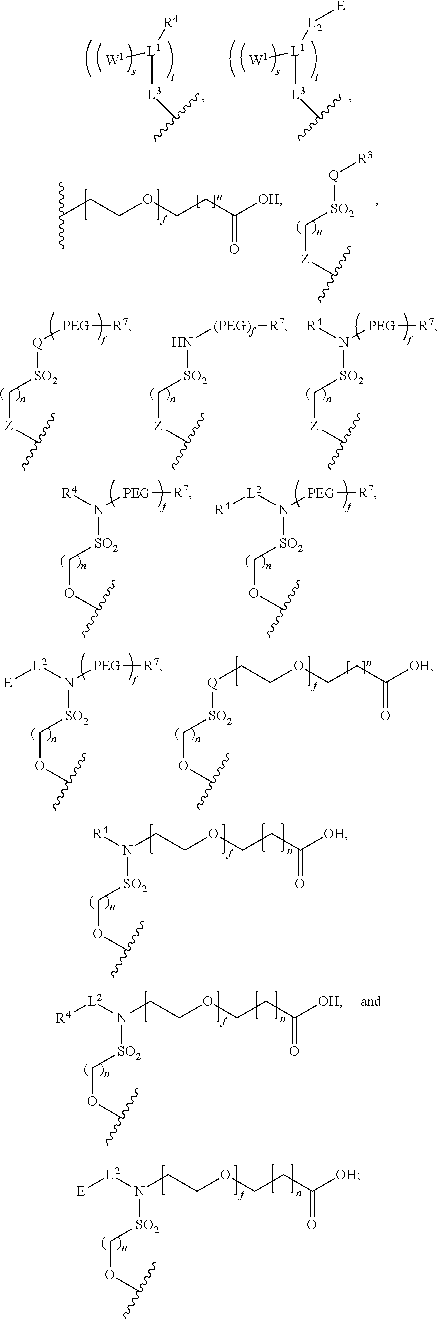
each R² is independently selected from the group consisting of a water-solubilizing moiety, a linker moiety, H, alkyl, alkene, alkyne, cycloalkyl, haloalkyl, alkoxy, (hetero)aryloxy, aryl, heteroaryl, (hetero)arylamino, a PEG group, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, 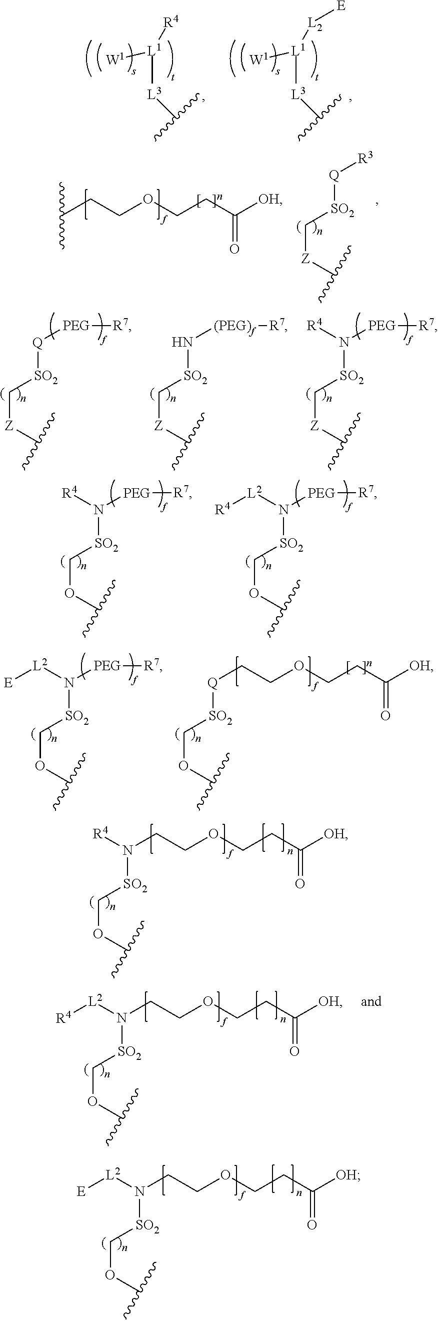
each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore and a PEG group;
each Z is independently selected from the group consisting of CH₂, CHR⁴, O, NR⁴, and NH;
each Q is independently selected from the group consisting of a bond, NH, NR⁴, C₁-C₁₂ alkylene, CHR⁴, and CH₂;
each R⁴ is independently selected the group consisting of H, a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OR⁹, wherein each R⁹ is C₁-C₈ alkyl, x′ is independently an integer from 0-20 and each y′ is independently an integer from 0-50, Z—(CH₂)ₙ—SO₂-Q-R³, a C₂-C₁₈ (hetero)aryl group, amide, amine, carbamate, carboxylic acid, carboxylate ester, maleimide, activated ester, N-hydroxysuccinimidyl, hydrazine, hydrazone, azide, aldehyde, thiol, and protected groups thereof;
each W¹ is independently a water-solubilizing moiety;
L, L¹, L², and L³ are each independently selected linker moieties;
each E is independently selected from the group consisting of a chromophore, a functional moiety, a substrate, and a binding partner;
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester and —OC₁—C₁₂ hydroxy;
at least one of R¹, R², R³, or R⁴ comprises a water-solubilizing moiety;
each M¹ is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted;
each optional M² is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than M¹, and wherein M² and M¹ are evenly or randomly distributed along the polymer main chain;
each optional linker L is independently a linker moiety;
G¹ and G² are each independently selected from the group consisting of an unmodified polymer terminus and a modified polymer terminus, optionally conjugated to E;
a, c, d, and e define the mol % of each unit within the structure which each can be evenly or randomly repeated along the polymer main chain and where a is a mol % from 10 to 100%, c is a mol % from >0 to 90%, each d is a mol % from 0 to 90%, and each e is a mol % from 0 to 25%;
each b is independently 0 or 1;
each f is independently an integer from 0 to 50;
m is an integer from 1 to about 10,000;
each n is independently an integer from 1 to 20;
s is 1 or 2; and
t is 0, 1, 2, or 3,
optionally further comprising a chromophore covalently attached to the polymer in energy-receiving proximity therewith.

2. The UV-absorbing polymer dye of claim 1, wherein G¹ and G² are each independently selected from the group consisting of hydrogen, halogen, alkyne, halogen substituted aryl, silyl, diazonium salt, triflate, acetyloxy, azide, sulfonate, phosphate, boronic acid substituted aryl, boronic ester substituted aryl, boronic ester, boronic acid, optionally substituted aryl, optionally substituted heteroaryl, optionally substituted dihydrophenanthrene (DHP), and optionally substituted fluorene, wherein the substituted aryl, heteroaryl, fluorene, or DHP are substituted with one or more pendant chains terminated with a functional group selected from the group consisting of amine, carbamate, carboxylic acid, carboxylate, maleimide, activated ester, N-hydroxylsuccinimidyl, hydrazine, hydrazide, hydrazone, azide, alkyne, aldehyde, thiol, and protected groups thereof, optionally conjugated to E.

3. (canceled)

4. The UV-absorbing polymer dye of claim 1, wherein each optional linker L is independently selected from the group consisting of an aryl or heteroaryl group evenly or randomly distributed along the polymer main chain and that is substituted with one or more pendant chains terminated with a functional group selected from amine, carbamate, carboxylic acid, carboxylate, maleimide, activated ester, N-hydroxysuccinimidyl, hydrazine, hydrazide, hydrazone, azide, alkyne, aldehyde, thiol, and protected groups thereof optionally conjugated to E, optionally wherein each optional linker L is independently selected from the group consisting of 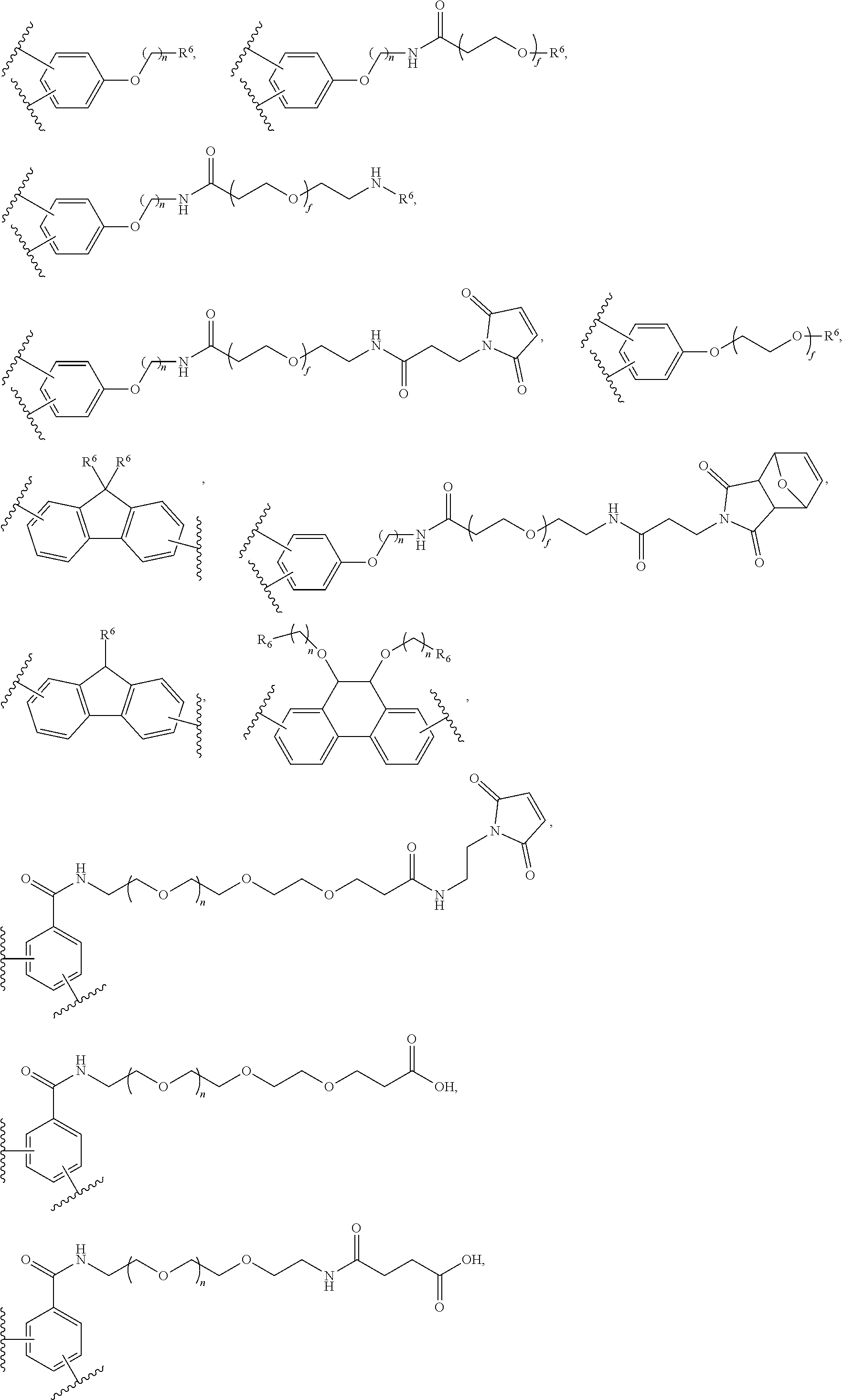 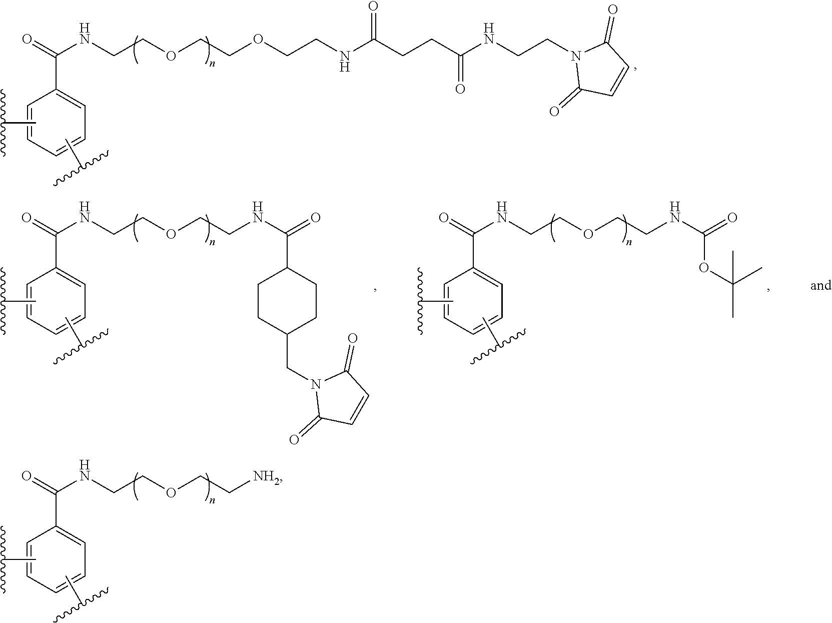
wherein each R⁶ is independently selected from the group consisting of H, OH, SH, NHCOO-t-butyl, (CH₂)ₙCOOH, (CH₂)ₙCOOCH₃, (CH₂)ₙNH₂, (CH₂)ₙNH—CH₂)ₙ—CH₃, (CH₂)ₙNHCOOH, (CH₂)ₙNHCO—(CH₂)ₙ—CO—(CH₂)ₙ—CH₃, (CH₂)ₙNHCOO—(CH₂)ₙ—CH₃, (CH₂)ₙNHCOOC(CH₃)₃, (CH₂)ₙNHCO(C₃-C₁₂)cycloalkyl, (CH₂)ₙNHCO(CH₂CH₂O)_{f}, (CH₂)ₙNHCO(CH₂)ₙCOOH, (CH₂)ₙNHCO(CH₂)ₙCOO(CH₂)ₙCH₃, (CH₂)ₙ(OCH₂CH₂)_{f}OCH₃, N-maleimide, halogen, C₂-C₁₂ alkene, C₂-C₁₂ alkyne, C₃-C₁₂ cycloalkyl, C₁-C₁₂ halo alkyl, C₁-C₁₂ (hetero)aryl, C₁-C₁₂ (hetero)arylamino, optionally substituted benzyl, halogen, hydroxyl, C₁-C₁₂ alkoxy, (OCH₂CH₂)_{f}OCH₃,

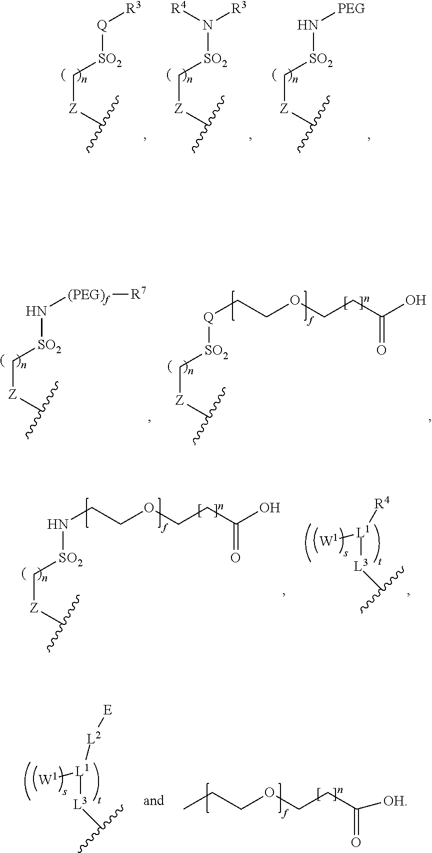

5. (canceled)

6. The UV-absorbing polymer dye of claim 1, wherein the polymer has the structure of Formula II or Formula III: 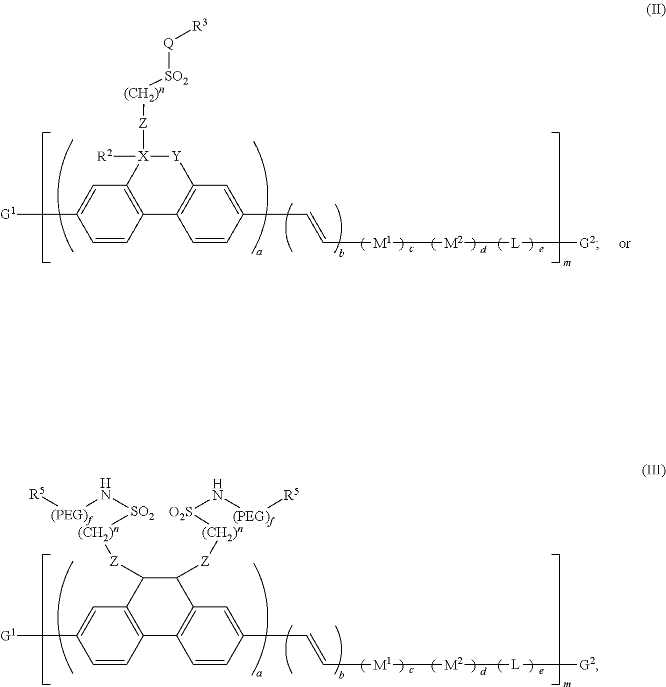
wherein each f is independently an integer from 0 to 50 and each R⁵ is independently selected from the group consisting of H, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, and C₁-C₁₂ alkoxy.

7. (canceled)

8. A UV-absorbing co-polymer dye comprising the polymer of claim 1, optionally wherein the polymer is a copolymer having the structure of Formula V: 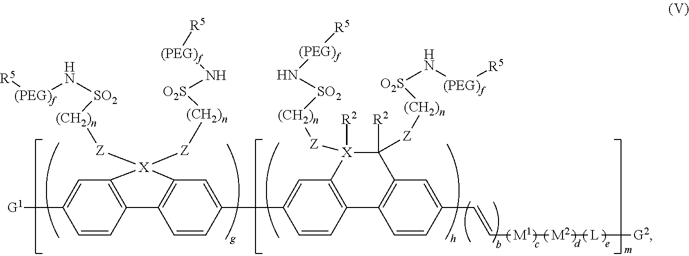
wherein g and h together is a mol % from 10 to 100%, each f is independently an integer from 0 to 50, and each R⁵ is independently selected from the group consisting of H, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, and C₁-C₁₂ alkoxy.

9. (canceled)

10. The UV-absorbing polymer dye of claim 1, wherein the polymer comprises the structure of Formula IX or Formula XIV: 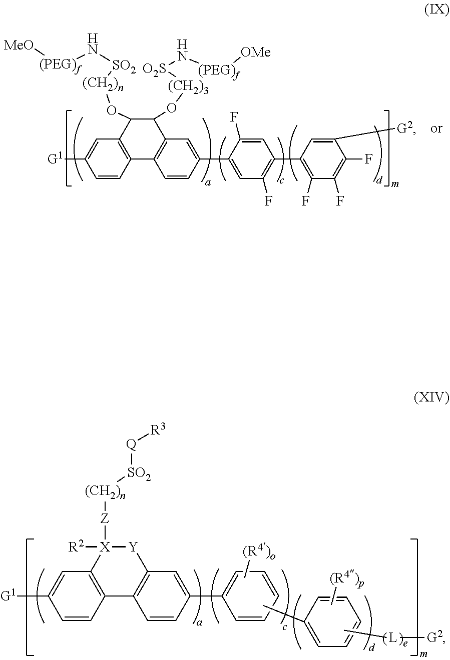
wherein
each R^{4′} is independently selected from F, Cl, —CH₃, —CF₃, and —(OCH₂CH₂)_{f}OR⁹;
each R^{4′} is independently selected from F, Cl, —CH₃, —CF₃, and —(OCH₂CH₂)_{f}OR⁹;
each R⁹ is C₁-C₈ alkyl;
each f is independently an integer from 0 to 50, 11 to 40, or 10 to 20;
each o is independently an integer selected from 1, 2, 3, or 4; and
each p is independently an integer selected from 1, 2, 3, or 4.

11.-12. (canceled)

13. The UV-absorbing polymer dye claim 1, wherein each M¹ is independently selected from the group consisting of: 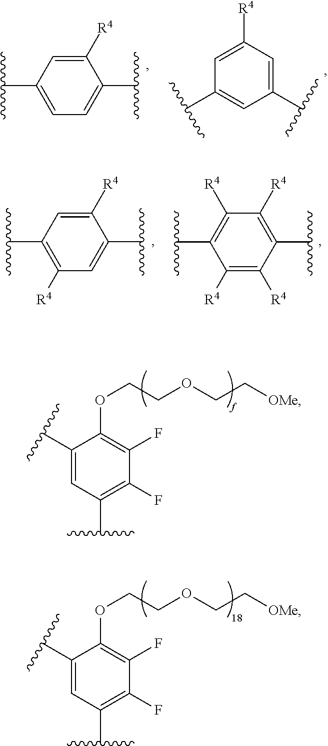 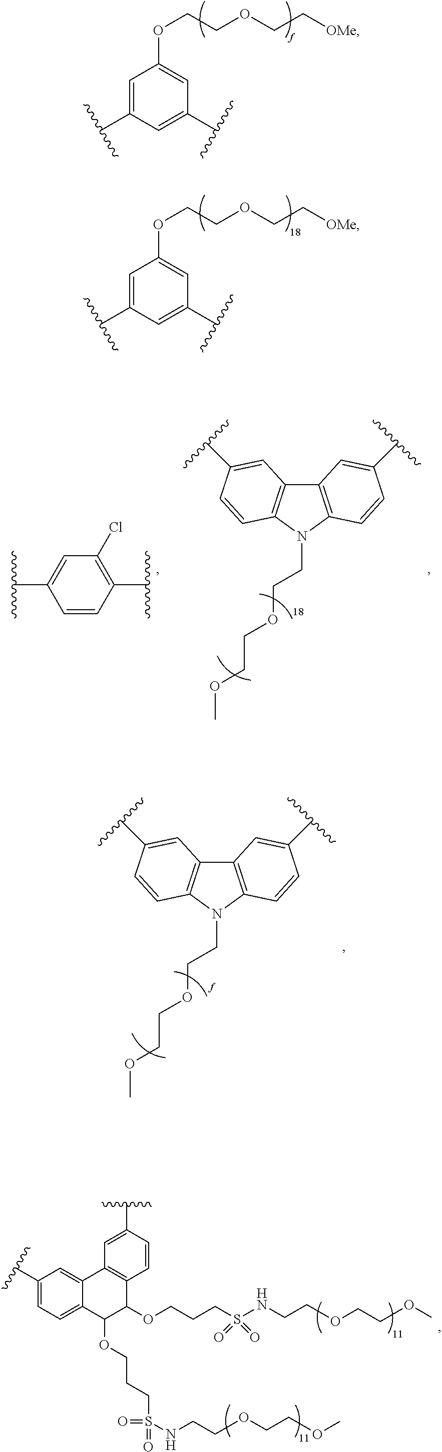 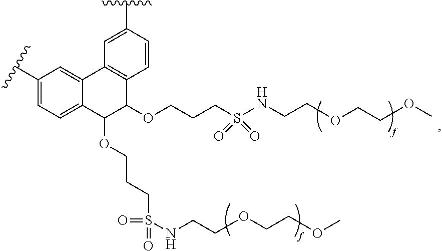 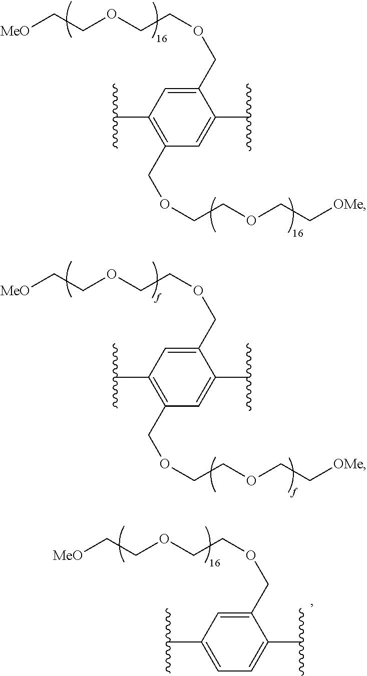 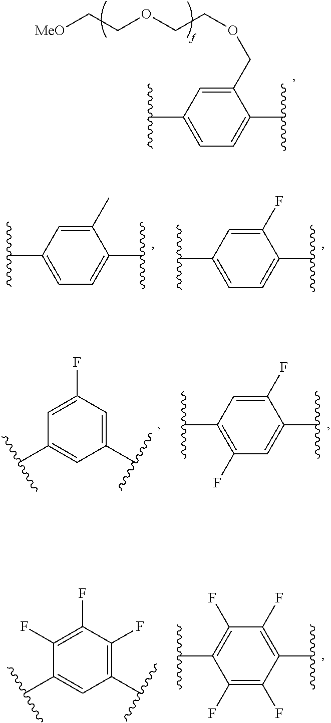
a fluorine-substituted arylene having 1-4 fluorine substituents, and a halide-, MeO-PEG-CH₂—, and/or a MeO-PEG-substituted arylene that is optionally further substituted,
wherein each f is independently an integer from 0 to 50, 10 to 20, or 11 to 18.

14.-15. (canceled)

16. The UV-absorbing polymer dye of claim 1, wherein each M² is independently selected from the group consisting of a halide-, MeO-PEG-CH₂—, and/or MeO-PEG-substituted arylene that is optionally further substituted, a fluorine-substituted arylene having 1-4 fluorine substituents, 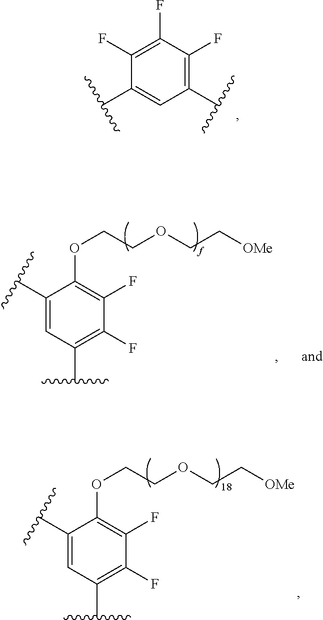
wherein each f is independently an integer from 0 to 50, optionally where M¹ and M² are different.

17.-19. (canceled)

20. The UV-absorbing polymer dye of claim 1, wherein a molar ratio of M¹ to M² groups is 0.5:1 to 1.5:1.

21. (canceled)

22. The UV-absorbing polymer dye of claim 1, wherein the polymer has
an absorption maximum in a range of from 300 nm to 400 nm, from 320 nm to 400 nm, from 320 nm to 380 nm, or from 350 nm to 400 nm, and
an emission maximum in a range of from 380 nm to 1000 nm, from 380 nm to 800 nm, or from 380 nm to 430 nm.

23. The UV-absorbing polymer of claim 1, comprising a binding partner covalently linked to the polymer, wherein the binding partner is a protein, peptide, affinity ligand, antibody, antibody fragment, sugar, lipid, nucleic acid, or an aptamer.

24.-26. (canceled)

27. The UV-absorbing polymer dye of claim 1, wherein the UV-absorbing polymer dye is a water-soluble UV-absorbing polymer dye.

28. A method for detecting an analyte in a sample comprising:
contacting a sample that is suspected of containing the analyte with the UV-absorbing polymer according to claim 23 to form a fluorescent polymer dye conjugate complex with the analyte;
applying a light source to the sample that can excite the at least one fluorescent polymer dye conjugate complex; and
detecting light emitted from the fluorescent polymer dye conjugate complex, optionally wherein the method is configured for flow cytometry, or wherein the method is configured as an immunoassay.

29.-32. (canceled)

33. A composition for use with at least one fluorescent polymer dye conjugated to a binding partner for use in staining a biological sample, the composition comprising:
at least one UV-absorbing polymer dye, UV-absorbing tandem dye, or quenched UV polymer dye; optionally wherein the UV-absorbing polymer dye, UV-absorbing tandem dye, or quenched polymer dye comprises a structure according to Formula I: 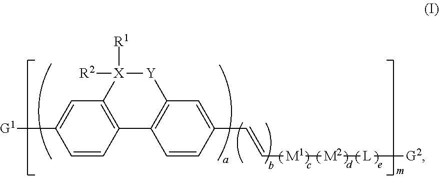
wherein
each X is independently selected from the group consisting of C and Si;
each Y is independently selected from the group consisting of a bond, CR¹R², CHR¹, CHR², SiR², SiHR¹, and SiR¹R², and when Y is a bond X is directly bonded to both rings;
each R¹ is independently selected from the group consisting of a water-solubilizing moiety, a linker moiety, alkyl, alkene, alkyne, cycloalkyl, haloalkyl, (hetero)aryloxy, (hetero)arylamino, aryl, heteroaryl, a polyethylene glycol (PEG) group, carboxylic acid, ammonium alkyl salt, ammonium alkyloxy salt, ammonium oligoether salt, sulfonate alkyl salt, sulfonate alkoxy salt, sulfonamido oligoether, sulfonamide, sulfinamide, phosphonamidate, phosphinamide, 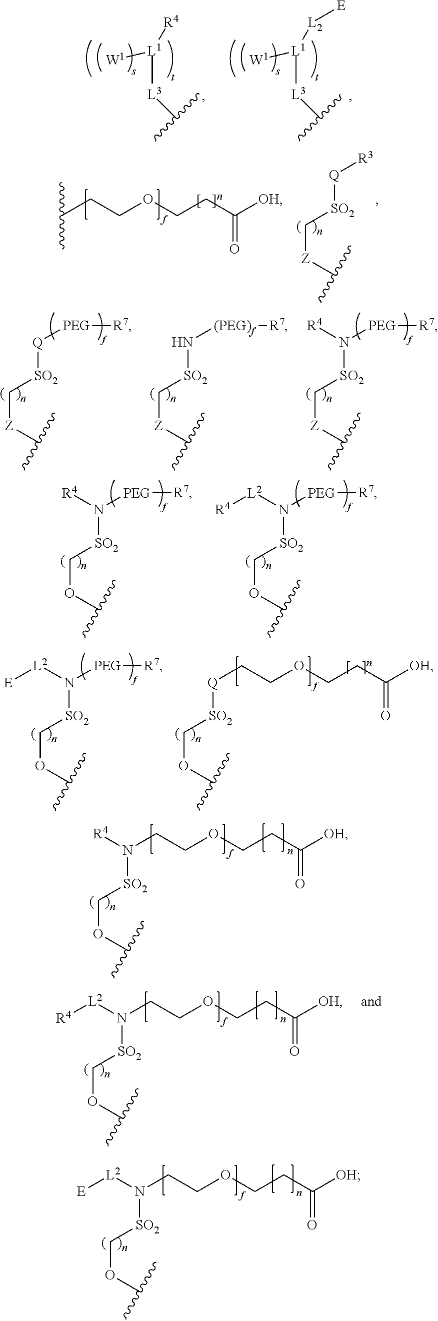
each R² is independently selected from the group consisting of a water-solubilizing moiety, a linker moiety, H, alkyl, alkene, alkyne, cycloalkyl, haloalkyl, alkoxy, (hetero)aryloxy, aryl, heteroaryl, (hetero)arylamino, a PEG group, sulfonamide-PEG, phosphoramide-PEG, ammonium alkyl salt, ammonium alkyloxy salt, ammonium oligoether salt, sulfonate alkyl salt, sulfonate alkoxy salt, sulfonate oligoether salt, sulfonamido oligoether, sulfonamide, sulfinamide, phosphonamidate, phosphinamide, 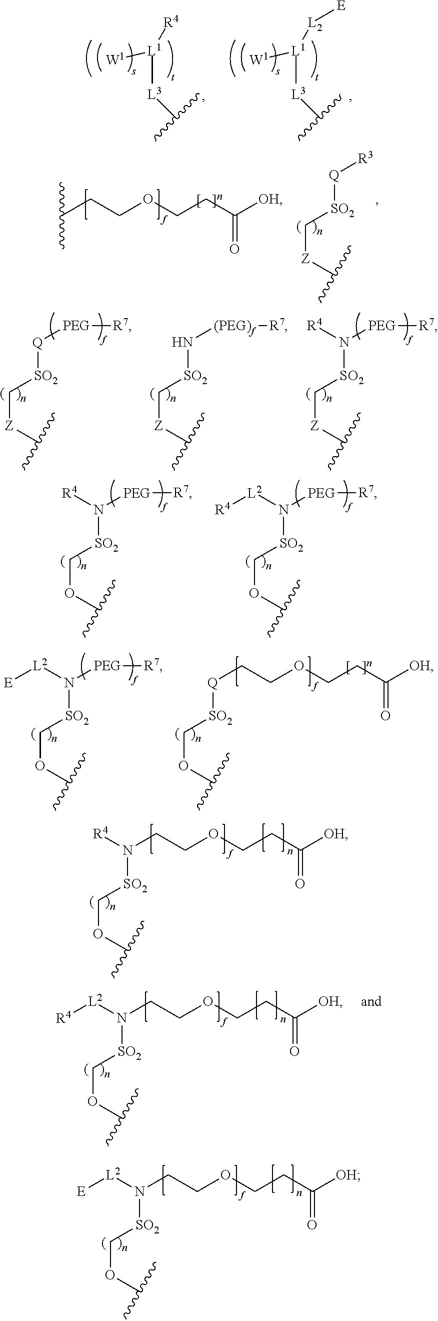
each R³ is independently selected from the group consisting of H, alkyl, alkene, alkyne, cycloalkyl, haloalkyl, alkoxy, (hetero)aryloxy, aryl, (hetero)arylamino, a water-solubilizing moiety, a chromophore and a PEG group;
each Z is independently selected from the group consisting of CH₂, CHR⁴, O, NR⁴ and NH;
each Q is independently selected from the group consisting of a bond, NH, NR⁴, C₁-C₁₂ alkylene, CHR⁴, and CH₂;
each R⁴ is independently selected the group consisting of H, a PEG group, a water-solubilizing moiety, a linker moiety, a chromophore, a linked chromophore, a functional group, a linked functional group, a substrate, a linked substrate, a binding partner, a linked binding partner, a quenching moiety, L²-E, halogen, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, (CH₂)_{x′}(OCH₂—CH₂)_{y′}OR⁹, wherein each R⁹ is C₁-C₈ alkyl, x′ is independently an integer from 0-20 and each y′ is independently an integer from 0-50, Z—(CH₂)ₙ—SO₂-Q-R³, a C₂-C₁₈ (hetero)aryl group, amide, amine, carbamate, carboxylic acid, carboxylate ester, maleimide, activated ester, N-hydroxysuccinimidyl, hydrazine, hydrazone, azide, aldehyde, thiol, and protected groups thereof;
each W¹ is independently a water-solubilizing moiety:
L, L¹, L², and L³ are each independently selected linker moieties:
each E is independently selected from the group consisting of a chromophore, a functional moiety, a substrate, and a binding partner:
each R⁷ is independently selected from the group consisting of H, hydroxyl, C₁-C₁₂ alkyl, C₂-C₁₂ alkene, C₂-C₁₂ alkyne, C₃-C₁₂ cycloalkyl, C₁-C₁₂ haloalkyl, C₁-C₁₂ alkoxy, C₂-C₁₈ (hetero)aryloxy, C₂-C₁₈ (hetero)arylamino, C₂-C₁₂ carboxylic acid, C₂-C₁₂ carboxylate ester and —OC₁—C₁₂ hydroxy:
at least one of R¹, R², R³, or R⁴ comprises a water-solubilizing moiety;
each M¹ is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted:
each optional M² is independently selected from the group consisting of an R⁴- and/or trifluoromethyl-substituted arylene that is optionally further substituted, an R⁴- and/or trifluoromethyl-substituted heteroarylene that is optionally further substituted, an R⁴- and/or trifluoromethyl-substituted 9,10-dihydrophenanthrene that is optionally further substituted, and a binaphthyl that is optionally substituted, wherein M² has a different structure than M¹, and wherein M² and M¹ are evenly or randomly distributed along the polymer main chain:
each optional linker L is independently a linker moiety;
G¹ and G² are each independently selected from the group consisting of an unmodified polymer terminus and a modified polymer terminus, optionally conjugated to E;
a, c, d, and e define the mol % of each unit within the structure which each can be evenly or randomly repeated along the polymer main chain and where a is a mol % from 10 to 100%, c is a mol % from >0 to 90%, each d is a mol % from 0 to 90%, and each e is a mol % from 0 to 25%;
each b is independently 0 or 1;
each f is independently an integer from 0 to 50:
m is an integer from 1 to about 10,000:
each n is independently an integer from 1 to 20;
s is 1 or 2; and
t is 0, 1, 2, or 3;
a nonionic surfactant; and
a biological buffer,
wherein the composition reduces non-specific binding of the at least one fluorescent polymer dye conjugate, when compared to the at least one fluorescent polymer dye conjugate in the absence of the composition.

34. The composition of claim 33, wherein the quenched UV polymer dye comprises the UV-absorbing polymer dye comprising at least one quenching moiety, optionally 1-30, 2-20, or 2.5-10 quenching moieties, optionally wherein the quenching moieties are selected from the group consisting of DABCYL, DABSYL, BHQ1, BHQ0, DDQI, EDQ, QSY7, QSY9, QSY35, TAMRA, Dabcyl Q, Dabcyl plus, 490Q, 425Q, and 505Q.

35. (canceled)

36. The composition of claim 33, wherein the nonionic surfactant is a poly(ethylene oxide)-poly(propylene oxide)-poly(ethylene oxide) triblock copolymer, optionally wherein the nonionic surfactant comprises a structure according to formula XII 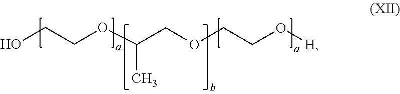
wherein each a is independently in the range of 2-130 and b is in the range of 15-67.

37. (canceled)

38. The composition of claim 33, further comprising an additional additive selected from the group consisting of protein stabilizer, preservative, and further surfactant, optionally wherein the further surfactant is selected from the group consisting of zwitterionic surfactant and ionic surfactant.

39. (canceled)

40. The composition of claim 33, wherein the composition comprises a plurality of fluorescent polymer dye conjugates, and the composition substantially reduces the non-specific binding between the plurality of fluorescent polymer dye conjugates.

41. A method for detecting an analyte in a sample comprising:
adding at least one polymer dye conjugated to a binding partner to a composition according to claim 33 to form a polymer dye conjugate composition;
contacting a biological sample that is suspected of containing an analyte with the polymer dye conjugate composition to form a fluorescent polymer dye conjugate complex with the analyte;
applying a light source to the sample that can excite the at least one fluorescent polymer dye conjugate complex; and
detecting light emitted from the fluorescent polymer dye conjugate complex, optionally wherein the biological sample is selected from the group consisting of blood, bone marrow, spleen cells, lymph cells, bone marrow aspirates, urine, serum, saliva, cerebral spinal fluid, urine, amniotic fluid, interstitial fluid, feces, mucus, or tissue.

42.-49. (canceled)

50. The method according to claim 41, wherein the detecting light further comprises analyzing by flow cytometry to obtain a first flow cytometry plot, wherein the first flow cytometry plot exhibits one or more of the group consisting of:
decreased non-specific interaction of polymer dye conjugates; and
decreased aggregation of polymer dye conjugates.
when compared to a second flow cytometry plot obtained comprising contacting the biological sample with a composition without the nonionic surfactant and without the UV-absorbing polymer dye or quenched UV polymer dye.

51. A kit comprising the composition according to claim 33, wherein the kit comprises a container comprising the composition; and optionally the at least one fluorescent polymer dye conjugate.

52. A kit comprising the UV-absorbing polymer dye according to claim 1, wherein the kit comprises the UV-absorbing polymer dye in at least one container.

53. A kit comprising the UV-absorbing polymer dye according to claim 23, wherein the kit comprises the UV-absorbing polymer dye in at least one container.
